(19) United States
(12) Patent Application Publication
(10) Pub. No.: US 2025/0205374 A1
(43) Pub. Date: Jun. 26, 2025

(54) METHOD FOR PROVIDING A LABELED SINGLE ISOMERIC CHEMICAL ENTITY TARGETING VECTOR BASED ON THE USE OF AN ISOMER-FREE DIENOPHILE

(71) Applicant: UNIVERSITY OF COPENHAGEN, Copenhagen K (DK)

(72) Inventors: Matthias Manfred HERTH, Malmö (SE); Umberto Maria BATTISTI, Copenhagen Ø (DK); Christian Bernard Matthijs POULIE, Bunkeflostrand (SE)

(21) Appl. No.: 18/844,866

(22) PCT Filed: Mar. 8, 2023
(86) PCT No.: PCT/EP2023/055930

§ 371 Date: Sep. 6, 2024

(30) Foreign Application Priority Data
Mar. 8, 2022 (EP) 22160909.2

(51) Int. Cl. A61K 51/10; A61K 51/08

(52) U.S. Cl. CPC A61K 51/1093; A61K 51/088

(57) ABSTRACT

The present disclosure regards a method for providing labeled single isomeric chemical entity targeting vectors suitable for providing targeting vectors. The method applies specific combinations between a diene and a dienophile with complementary inverse electron demand Diels-Alder cycloaddition reactivity, which upon ligation, followed by oxidation, will form compounds of a single isomeric form. The labeled single isomeric chemical entity targeting vectors are for use in therapy, radiotherapy, theranostics, diagnostics, and imaging. The method applies click chemistry wherein one chemical entity which is conjugated to a label is clicked together with a second chemical entity with complementary inverse electron demand Diels-Alder cycloaddition reactivity which is conjugated to a targeting vector followed by a rapid oxidation, to form a single isomeric compound.

### FIELD

The present invention relates to a method for providing a labeled single isomeric chemical entity targeting vectors, the targeting vectors obtained and the uses of the targeting vectors. The labeled single isomeric chemical entity targeting vectors can be used in therapy such as radiotherapy, diagnostics, imaging, and other photochemistry methods.

### BACKGROUND

Labeled targeting vectors based on click chemistry between dienes and dienophiles are used both for imaging purposes such as diagnostics and other photochemistry imaging methods and in therapy. Such targeting vectors have for instance been labeled with radiolabels than can be applied in diagnostics and/or in therapy. The specific use depends on the identity of the radiolabeling used because different radionuclides provide for different purposes. The specific use moreover depends on the specific target that the vector is directed at. Several combinations of radiolabels and vectors are applied presently in diagnosis, therapy, theranostic and imaging. Different chemical entities connecting the radiolabeled entity with the target directed entity exists, the present invention is based on click-chemistry wherein a diene and a corresponding dienophile is ligated thereby bridging the radiolabel and the target directed entity.

The term “click chemistry” refers to a class of reactions that are fast, simple to use, versatile, chemoselective, and give high product yields. These reactions have found application in a wide variety of research areas, including materials science, polymer chemistry, and pharmaceutical sciences. Radiochemistry is one of the fields that showed the true potential of click chemistries as for example disclosed in Zeng et al, Journal of Nuclear Medicine, 54, 829-832, 2013. Essentially, the selectivity, ease, rapidity, and modularity of click ligations make them nearly ideally suited for the construction of radiotracers, a process that usually involves working with biomolecules in aqueous conditions with fast decaying radioisotopes. Among the different click chemistries, one of the most suited and utilized for radiolabeling is the tetrazine ligation.

The tetrazine ligation is a click reaction which is characterized by the formation of covalent bonds between a 1,2,4,5-tetrazines (Tz) and typically a trans-cyclooctene (TCO). The reaction is initiated by an inverse electron-demand Diels-Alder reaction, followed by a retro-Diels-Alder reaction, driven by the expulsion of N₂. The tetrazine ligation is among the fastest known chemical ligations, with second order rate constants up to 10⁶ M⁻¹s⁻¹ in acetonitrile at 25° C. This stands in contrast to other known click reactions, such as the Staudinger ligation (0.003 M⁻¹s⁻¹) and strain-promoted azide-alkyne cycloaddition (SPAAC) (0.1 M⁻¹s⁻¹). For this reason, and because of its specificity, the Tz ligation is ideally suited for synthon-based labeling. One major drawback of the conventional ligation of a Tz with a TCO is that the ligation takes place without any regio-specificity. Moreover, the conventional ligation of a Tz with a TCO gives rise to several tautomeric forms. This means that conventional ligation of a Tz with a TCO typically results in complex reaction mixtures of at least sixteen unique isomeric products (Scheme 1). Such mixtures of isomeric products cannot be applied directly for pharmaceutical purposes because the particular isomeric form of the compound influences the pharmacokinetics of the therapeutic agent and any potential toxicological effects of the individual isomeric forms cannot be distinguished. While the multiple dihydropyridazines that are obtained from conventional ligation may slowly auto-oxidize to the corresponding pyridazines over the course of several hours to several days, thereby slowly reducing the number of tautomeric forms of the products (WO2012/121746), the number of regio-isomeric and stereo-isomeric products are not reduced by this slow oxidation transformation. Means for speeding up the transformation from dihydropyridines to pyridazines have been described (Karaki Fumika et al, Tetrahedron, 97, 132411, 2021; Keindnen et al., ACS Medicinal Chemistry Letters, 7, 62-66, 2015), however, in none of reported cases was the oxidation completed within two hours, which is required if the pyridazines are to be used as radiopharmaceuticals. WO2017/059397, WO2020/242948, Syvsnen et al., ACS Chemical Neuroscience, 11, 4460-4468, 2020, and WO2012/121746 discloses ligations between tetrazines and TCO's, which will inevitably provide several isomeric chemical entities.

Alternatively, pyridazines can be prepared via the ligation of a Tz to a strained cyclic alkyne, however this reaction suffers from slow second order rate constants.

Radiopharmaceuticals are increasingly used in theranostic, especially within oncology, both for diagnostic imaging and for targeted radionuclide therapy. Positron emission tomography (PET) is the gold standard in nuclear imaging with better resolution and quantification than other modalities. 2,200,800 clinical PET scans were performed in 2019 in the US alone. Targeted radionuclide therapy is more effective at treating cancer than many state-of-the-art chemotherapies. It also has the advantage over external beam radiotherapy (e.g. “gamma knife”) in that it offers a way to confine the delivered dose to the tumor and its immediate surrounding area, which makes particular sense in the radiotherapy of micrometastatic disease. The combination of both diagnostic imaging and targeted radiotherapy can be used in “theranostics”, a concept with powerful application in personalized medicine, with respect to patient selection, dose-finding and therapy response monitoring. A theranostic pair is two radionuclides, which can be substituted with each other, without changing the pharmacokinetics of the radiopharmaceutical, but shifting their application between diagnostic imaging and radionuclide therapy.

The two most widely used Diagnostic Imaging methods are the nuclear based PET and SPECT. Both methods rely on the combination of radionuclides with vectors that specifically target cancer cells. In imaging, such radiolabeled vectors are referred to as “radiotracers”. Radiotracers are accumulated in tumor lesions, the location of which can then be visualized by detecting the emitted radiation. PET is strongly favored in oncology, while SPECT is dominant in cardiology and for producing bone scans and certain other specialized organ scans. Globally, the ratio of SPECT cameras to PET cameras used in hospitals is circa 5:1.

Single-photon emission computed tomography (SPECT) is the older of the two methods. SPECT imaging employs radionuclides emitting gamma photons, typically in the 100-200 keV range. A series of 2D projection images of radiotracer distribution in the body are acquired by one of more gamma cameras from multiple angles. These projection images are then assembled to produce a 3D image.

Positron emission tomography (PET) is currently considered the most advanced form of nuclear imaging. The key use of PET in oncology is diagnosis and treatment monitoring, especially of metastatic cancer. Compared to previous modalities, notably SPECT, PET offers improved resolution and sensitivity, and generally higher quality images. These properties are especially relevant in the detection, and subsequent treatment, of very small metastases. New innovations, notably total-body PET dramatically increase sensitivity and detects more metastases and are expected to further strengthen the advantageous position of PET in the modern clinic. PET relies on the use of radionuclides that emit positrons upon their decay. These positrons travel a limited distance, and then undergo annihilation with an electron in the surrounding medium. This produces two annihilation photons, each of 511 keV, which are emitted in opposite directions. These photons can be detected by a PET scanner. The most optimal radionuclide for PET is fluorine-18 (¹⁸F). With a decay half-life of 110 minutes and 97% positrons emitted per decay, ¹⁸F is close to ideal for clinical PET applications. This holds true especially for small molecular and peptide-based radiopharmaceuticals, which represent the vast majority of relevant PET tracers. Of equal importance, ¹⁸F can be practically produced in enormous quantities (>300 doses per production) on standard biomedical cyclotrons, which are readily available throughout most of the world, with more than 200 present in Europe alone. Accordingly, ¹⁸F does not share the concerns for sufficient supply associated with its closest competitor, the generator-produced radiometal gallium-68 (⁶⁸Ga). In addition, the lower positron energy of ¹⁸F provides higher resolution images. Compared to the current diagnostic radionuclide of most widespread use, the SPECT radionuclide technetium-99m, ¹⁸F offers the highest quality images through its status as a PET radionuclide. Accordingly, ¹⁸F is poised as the key diagnostic radionuclide of the future.

Radioactive iodine is widely used in SPECT imaging. Traditionally, iodine-131 (¹³¹I) was used, but nowadays, clinical SPECT scans are typically done with iodine-123 (¹²³I). This is due to the shorter half-life of ¹²³I (T½=13.2 hours) which together with its lack of beta minus emission offers a more favorable radiotoxicity profile than ¹³¹I Its gamma photon energy of 159 keV is excellently suited for clinical SPECT imaging. Due to the intrinsic accumulation of iodine in the thyroid, ¹²³I in its free form is widely used for imaging thyroid disease. As a component of SPECT radiotracers, ¹²³I is for example used in the imaging agents MIBG (oncology) and ioflupane (CNS). ¹²³I forms a theranostic pair with the clinically used beta minus emitting therapeutic radionuclide ¹³¹I and the investigational Auger electron radiotherapeutic ¹²⁵I. lodine-123 is produced in a cyclotron by proton irradiation of xenon in a capsule and is commercially available.

lodine-124 (¹²⁴I) can be used for PET imaging. It is usually produced in a cyclotron by bombardment of enriched tellurium-124. However, the imaging characteristics of ¹²⁴I are not ideal. It has a complex decay scheme with many high energy γ-emissions. Only 23% of its decay leads to positron emissions.

Astatine-211 (²¹¹At) is primarily a therapeutic nuclide, which emits alpha-particles upon decay. Alpha particles are absorbed in just 100 μm of tissue and cannot be detected by external scanners. However, one of the decay branches of ²¹¹At also generates X-rays in the range of 70-90 keV, which can be imaged with a gamma-camera or a SPECT scanner. Therefore, the distribution of ²¹¹At-labeled radiopharmaceuticals within the patient can be clinically assessed by SPECT imaging.

There are, however, challenges to introduce above mentioned radionuclides into molecules which limits the practical use of radionuclide-based therapy, diagnostic, and imaging.

Most of the PET, SPECT and therapeutic radionuclides mentioned above are radiometals. Conventional method of introducing radiometals into the vector molecules is the use of chelator groups that form coordinate bonds with the radiometal atom. The radiolabeling procedure typically involves mixing the radiolabeling precursor (vector with a chelator group attached) with radiometal ions and heating the mixture to allow the chelation reaction to proceed. Although chelation of radiometals is conceptually simple, it has a number of drawbacks, namely:
the radiolabeled product often cannot be separated from the unlabeled precursor, because the difference in physico-chemical properties is not significant;
chelation reaction is sensitive to trace metal impurities in solutions used for the radiolabeling, which makes upscaling problematic;
heating, which is necessary to overcome the activation barrier of the chelation reaction, may degrade temperature-sensitive vectors.

Compared to its radiometal alternatives ¹⁸F is a halogen and requires covalent bonding to targeting vectors. This stands in the contrast to the chelator-based labelling techniques utilized for radiometals. Covalent bonds are currently typically formed via direct nucleophilic displacement of a leaving group, such as triflate. The conditions for such chemistry are harsh, lengthy and poorly scalable, and therefore incompatible with many vectors, notably the peptide class, which is growing in importance. Small molecular radiopharmaceuticals containing radioiodine are typically prepared using either electrophilic destannylation or iodine-iodine exchange radiochemistry. The former is a mild, versatile and practical reaction, in which radioactive iodide is oxidized to a positively charged iodine species, which then replaces a leaving group, typically stannyl, in an aromatic substitution reaction. This reaction occurs at room temperature in often quantitative yield. Iodine-iodine isotopic exchange is used when high molar activity is not a concern and when substrates can withstand harsh conditions. The exchange occurs at elevated temperature with acid and copper as catalysts.

Like fluorine and iodine, astatine-211 is a halogen and can be attached to targeting vectors via covalent bonds. Aliphatic astatine-carbon bonds do not provide sufficient in vivo stability, whereby ²¹¹At is typically introduced onto aryl rings, forming astatoaryl moieties. Unlike iodine, astatine cannot be stably coupled to tyrosine residues of proteins: instead of binding to tyrosine, ²¹¹At has been found to form weak bonds with the sulfhydryl groups of cysteine. Therefore, ²¹¹At-labeling requires the synthesis of dedicated precursors exhibiting a suitable leaving group, such as trialkylstannyl, connected to an aryl ring. Standard ²¹¹At-labeling protocols use oxidation agents such as chloramine-T or N-chlorosuccinimide. Such agents can potentially degrade the biomolecules used as targeting vectors.

¹⁸F has long established itself as the best-in-class radionuclide for diagnostic PET imaging, while ²¹¹At is the most promising therapeutic radionuclide for alpha-therapy. Iodine radioisotopes ¹²³I, ¹²⁴I, ¹²⁵I and ¹³¹I are useful for SPECT imaging, PET imaging, Auger therapy and beta-therapy, respectively. Moreover, all three elements can be introduced into aryl rings forming fluoro/iodo/astatoaryl moieties with maximum structural similarity, which is essential for theranostic pairs. Nevertheless, there are no reports of combined use of ¹⁸F with either ²¹¹At or radioiodine ¹²³I, ¹²⁴I, ¹²⁵I, ¹³¹I, for theranostic applications.

One obstacle to the development of ¹⁸F/²¹¹At and ¹⁸F/¹²³I, ¹²⁴I, ¹²⁵I, ¹³¹I, theranostic pairs is the harsh labeling chemistry, as described above, which prevents direct regioselective labeling of biomolecular targeting vectors with these radionuclides. Another obstacle is that not all targeting vectors contain aryl moieties, so these must be introduced as prosthetic groups, also known as synthons.

Due to these challenges, synthon-based methods have been investigated for the preparation of radiopharmaceuticals. These methods involve the direct labeling of a separate intermediate compound (“synthon”), which is subsequently conjugated to the vector under mild conditions. Thus, the vector is not subjected to the harsh conditions of direct radiolabeling, although it does need to be modified by a chemical tag complementary to the radiolabeled synthon. What becomes crucial then, is the chemistry used to conjugate the synthon with the vector. This chemistry must be specific, compatible with pharmaceutically relevant aqueous media and high yielding, to avoid loss of radioactivity and minimize the need for subsequent purification. Critically, it must also be extremely fast, as procedures for preparing radiopharmaceuticals occur in nano- to micromolar concentrations and must proceed to completion or near completion within minutes, due to radionuclide decay. In light of that, click-chemistry has emerged as a strategic approach to radiolabel an array of targeting vectors such as mAbs, nanomedicines, peptides or small molecules.

Targeted radionuclide therapy (TRT) can be based on beta-emitters, Auger electron emitters and alpha-emitters.

Beta-particle emitting radionuclides (such as ⁹⁰Y, ¹⁷⁷Lu, ¹³¹I) decay via the emission of high-energy electrons (beta particles) which travel distances in the tissue of up to about 12 mm. The decay energy is deposited within the largest volume of tissue of the three therapy types mentioned here. Beta-emitters are thus suitable for the treatment of medium-sized tumors, where most of the dose will be absorbed by the cancer cells. For micrometastases or heterogeneous tumors however, even with perfect concentration of the radionuclide in and around the tumor, a large fraction of the irradiation dose is absorbed by surrounding healthy cells. Therefore, beta-emitters are not optimal for the treatment of micrometastases or heterogeneous tumors. This is an important drawback of beta-emitters, because micrometastases are one of the major causes of cancer recurrence and cancer mortality.

Alpha-emitters (such as ²²⁵Ac, ²¹At and ²¹²Pb) decay with the emission of alpha particles. Alpha-particles are much heavier than beta-particles, and their tracks are straight and short—on the order of 30-100 μm, in the order of the diameter of a handful of mammalian cells. Thus, all energy from a decay is delivered to just a few neighboring cells. Alpha-radiation possesses greater cytotoxicity, compared to beta-radiation and can be delivered to micro metastases in a highly focused manner. In a telling example of the advantages of alpha therapy, a patient with metastatic prostate cancer resistant to the flagship beta-therapy agent ¹⁷⁷Lu-PSMA achieved complete remission after three cycles of the alpha-therapy agent ²²⁵Ac-PSMA. Auger electron radiotherapy (AeRT) employs radionuclides that upon decay by electron capture (EC) or internal conversion (IC) emit a shower of extremely short ranged electrons. With advanced drug delivery technology, these specialized radionuclides can be delivered to the nuclei of cancer cells. Here, the emitted Auger electrons destroy the DNA and kill the cancer cells. Notably, the short range of the Auger electrons ensures that their energy is deposited mainly within the targeted cell, allowing for extremely localized therapy. Both¹²³I and ¹²⁵I have high Auger electron yields and are suitable for AeRT.

lodine-131 (¹³¹I) is a beta particle emitter that is widely used in clinical radionuclide therapy. It has a decay half-life of 8.0 days and a main emission of beta particles with a maximal energy of 606 keV at 90% abundance. These beta-particles have a maximum range in tissue of about 2 mm, enabling ¹³¹I to treat small to medium sized tumor lesions. It is widely used for thyroid ablation due to its intrinsic accumulation in thyroid tissue. In addition, a therapeutic variant of MIBG is available, radiolabeled with ¹³¹I, and ¹³¹I is used in radioimmunotherapy. It forms theranostic pairs with ¹²³I (SPECT) and ¹²⁴I (PET). Both iodine-123 and iodine-125 have substantial emission of Auger electrons, about 10 and 20 electrons, respectively. This makes them suitable for Auger electron radiotherapy, a currently investigational form of radionuclide therapy.

Astatine-211 is an alpha-emitting radionuclide with a half-life of 7.2 hours. Unlike most other alpha-emitters used for targeted alpha-therapy, ²¹¹At yields one α-particle per decay chain, which offers a number of translational advantages. First, there are limited toxic side-effects from radioactive daughter nuclides, which are released from the targeting vector as a result of the initial decay. Second, radiation dosimetry calculations are simplified. Moreover, due to the relatively short half-life of ²¹¹At (for a therapeutic nuclide), enhanced control of the radiation dose delivered to patients is possible.

Phase I/II Clinical studies with tumor-targeting ²¹¹At-labeled radiopharmaceuticals showed low acute toxicity and absence of radiation side-effect. Preclinical studies showed efficient tumor eradication by ²¹¹At in animal models. Hence, ²¹¹At is a highly promising therapeutic radionuclide.

However, in order for click-chemistry such as the Tz-TCO ligation to furnish end-products that are viable in a regulatory and commercial context, it is critical that only a single ligation product is produced. This is not possible using available methods because the ligation conventionally yields a large number of isomeric products that are impossible to separate, barring it from clinical translation due to toxicity concerns and unpredictable pharmacokinetics. The present invention provides a method wherein certain combinations of chemical entities with complementary inverse electron demand Diels-Alder cycloaddition reactivity, which upon ligation, followed by a rapid oxidation, will form a single compound. This means that only one isomeric product is obtained and accordingly no separation of isomeric products is required. The method advantageously enables radiolabeling, for example with ¹⁸F, ¹²³I, ¹²⁴I, ¹²⁵I or ¹³¹I, and ²¹¹At, of any tracer in unmatched efficiency and practicality.

### SUMMARY

The present invention provides a method for providing labeled single isomeric chemical entity targeting vectors. The method applies click chemistry wherein one chemical entity which is conjugated to a label is clicked together with a second chemical entity with complementary inverse electron demand Diels-Alder cycloaddition reactivity which is conjugated to a targeting vector followed by a rapid oxidation, to form a single isomeric compound. The advantage of the method is that one single isomeric end-product, within a minimum period of time will be provided, and thereby easing clinical translation and production costs.

The method for providing a labeled single isomeric chemical entity targeting vector comprises the following steps:
a) labeling a first chemical entity having inverse electron demand Diels-Alder cycloaddition reactivity and being conjugated to a pharmaceutic agent, an imaging agent, or a therapeutic agent, with a labeling agent; wherein the first chemical entity is selected from the group consisting of a symmetrical substituted diene wherein at least one of the symmetry planes passes through the nitrogen-nitrogen bonds of at least one tetrazine ring, an unsymmetrical substituted diene, and an isomer-free dienophile; and
b) ligating the labeled first chemical entity obtained in step a) with a second chemical entity having complementary inverse electron demand Diels-Alder cycloaddition reactivity and being conjugated to a targeting vector; wherein the second chemical entity is selected from the group consisting of a symmetrical substituted diene wherein at least one of the symmetry planes passes through the nitrogen-nitrogen bonds of at least one tetrazine ring, an unsymmetrical substituted diene and an isomer-free dienophile;
wherein the reaction kinetics for the inverse electron demand Diels-Alder cycloaddition between the first and second chemical entities has a minimum second order rate constant of 500 M⁻¹ s⁻¹ in phosphate-buffered saline (PBS) at 25° C., determined by stopped-flow spectrophotometry,
with the proviso that when the labeling agent in step a) is ⁹⁴Tc, ^{99m}Tc, ²¹¹At, ²²³Ra or ²²⁵Ac the labeling agent is conjugated to an unsymmetrical substituted diene; and
c) oxidizing the ligated labeled targeting vector obtained from step b) at a temperature ranging from 15° C. to 50° C. for up to 60 minutes by adding from 1 to 100 equivalents of chloranil, fluoranil, DDQ or NaNO₂.

This labeling agent can be any agent that is useful as a marker, an imaging agent, a therapeutic agent or a theranostic agent and includes radionuclides and fluorescent entities.

The targeting vector can be any suitable vector directed at a specific target and includes antibodies, nanobodies, polymers, nanomedicines, cells, proteins, peptides, and small molecules.

Several examples of symmetrical substituted dienes, unsymmetrical substituted dienes and isomer-free dienophiles suitable for this method is disclosed herein.

Suitable dienes include for example tetrazines. Suitable dienophiles include for example trans-cycloheptenes (TCH's), trans-cyclooctenes (TCO's) and trans-cyclononenes (TCN's).

The method of the present invention also include an embodiment wherein the first chemical entity and/or the second chemical entity is obtained from specific precursors. These precursors include precursors for obtaining symmetrical substituted dienes, for obtaining unsymmetrical substituted dienes and for obtaining isomer-free dienophiles, respectively.

The present invention moreover provides for use of the labeled single isomeric chemical entity targeting vectors obtained by the method in theranostic, therapy, radiotherapy, diagnostic and imaging.

### BRIEF DESCRIPTION OF DRAWINGS

FIG. 1: Scheme showing the synthesis of symmetrical tetrazines.

FIG. 2: Scheme showing an alternative synthesis of symmetrical tetrazines.

FIG. 3: Radio-HPLC of [¹⁸F]I at end of deprotection.

FIG. 4: shows the UV trace of [¹⁸F]XX.

FIG. 5: shows the radioactivity trace of [¹⁸F]XX.

FIG. 6: shows UV and radioactivity trace of [¹⁸F]XX—analytical HPLC

FIG. 7: UV trace of [¹⁸F]X—Semi-prep HPLC.

FIG. 8: Radioactivity trace of [¹⁸F]X—Semi-prep HPLC.

FIG. 9: UV and radioactivity trace of [¹⁸F]X—analytical HPLC.

FIG. 10: UV trace—Semi-prep HPLC of [¹⁸F]XI.

FIG. 11: radioactivity trace—Semi-prep HPLC of [¹⁸F]XI.

FIG. 12: UV and radioactivity trace of [¹⁸F]XI—analytical HPLC.

FIG. 13: Radio-HPLC of crude [¹²⁵I]XVII.

FIG. 14: UV and radioactivity trace—Semi-prep HPLC of [²¹¹At]XIV.

FIG. 15: radioactivity trace of purified [²¹¹At]XIV—analytical HPLC.

FIG. 16: Table showing results of the click experiments with radiolabeled tetrazines in Example 11.

FIG. 17: Scheme of click reaction performed with 27 and table of data from with click reaction performed with 27.

FIG. 18: Scheme of click reaction performed a Tz and two different TCOs and table of data from these click reactions.

FIG. 19: HPLC analysis after oxidation of the tetrazine-TCO pyridazine tested in Example 12

FIG. 20: Scheme of click reaction performed a [¹⁸F]Tz and four different TCOs and table of data from these click reactions

FIG. 21: Structures of vectors tested in Example 13.

FIG. 22: Table showing the results of the oxidation of vectors from Example 13.

### DETAILED DESCRIPTION OF THE INVENTION

The present invention provides in a first aspect a method for providing a labeled single isomeric chemical entity targeting vector. The method applies specific combinations between a diene and a dienophile with complementary inverse electron demand Diels-Alder cycloaddition reactivity, which upon ligation, followed by oxidation, will form compounds of a single isomeric form.

Either the diene or the dienophile is conjugated to an agent of interest such as a pharmaceutic agent, an imaging agent, or a therapeutic agent and labeled with a labeling agent. The compatible diene or dienophile, respectively, is conjugated to a targeting vector of interest.

The ligation between the diene and the dienophile is based in inverse electron demand Diels-Alder cycloaddition reactivity, and accordingly, the diene and the dienophile to be ligated must have complementary inverse electron demand Diels-Alder cycloaddition reactivity. Moreover, the ligation between the diene and the dienophile should have reaction kinetics with a minimum second order rate constant of 500 M⁻¹ s⁻¹ in PBS at 25° C. as determined by stopped-flow spectrophotometry in order to be of relevance to the present method. Ligations with second order rate constants below 500 M⁻¹ s⁻¹ in phosphate-buffered saline (PBS) at 25° C. will take too long time to provide the labeled targeting vectors because the radioactive labeling agent often have a limited period of time for use on imaging and/or therapy after the ligation step.

Second order rate constant can be measured by different means, but is typically measured by stopped flow spectrophotometry as for example described in (Chance, Rev. Sci. Instrum. 1951, 22, 619-627). Herein, the method described in Battisti et al. J. Med. Chem. 2021, 64, 20, 15297-15312 was applied.

In order to provide a labeled chemical entity or targeting vector that will result in only a single isomeric form when ligating the diene and the dienophile with complementary Diels-Alder cycloaddition reactivity, a combination of two requirements is necessary. The first requirement relates to selecting the structures of the diene and of the dienophile to be ligated. It has been found here that two ligation options are available for combining the diene and the dienophile in order to provide a combined (i.e., clicked) product that upon oxidation will provide a targeting vector in just one isomeric form: combination option i) ligation between an unsymmetrical substituted diene and an isomer-free dienophile; ligation combination option ii) ligation between a symmetrical substituted diene and an isomer-free dienophile.

The second requirement relates to the oxidation step. It is shown herein that not all oxidants are suitable for this method either because the oxidation is not efficient enough when working within the required time frame available or give side products or because the oxidation impacts on the structure of the targeting vector, thereby potentially preventing the labeled targeting vector from binding to its target. The oxidation efficiency of the present oxidation step is at least 90% i.e., at least 90% of the labeled and clicked targeting vector should be in a single isomeric form after the oxidation step. Oxidation conditions providing less than 90% of the product is in a single isomeric form, it will not be of sufficient purity for use in therapy/imaging/diagnosis and it will require additional toxicological studies.

It is shown herein that a suitable oxidation is performed at a temperature ranging from 15° C. to 50° C. for up to 60 minutes by adding from 1 to 100 equivalents of an oxidant selected from the group comprising chloranil, fluoranil, DDQ and NaNO₂.

Accordingly, the method for providing a labeled single isomeric chemical entity targeting vector comprises:
a) labeling a first chemical entity having inverse electron demand Diels-Alder cycloaddition reactivity and being conjugated to a pharmaceutic agent, an imaging agent, or a therapeutic agent, with a labeling agent; wherein the first chemical entity is selected from the group consisting of a symmetrical substituted diene wherein at least one of the symmetry planes passes through the nitrogen-nitrogen bonds of at least one tetrazine ring, an unsymmetrical substituted diene, or an isomer-free dienophile; and
b) ligating the labeled first chemical entity obtained in step a) with a second chemical entity having complementary inverse electron demand Diels-Alder cycloaddition reactivity and being conjugated to a targeting vector; wherein the second chemical entity is selected from the group consisting of a symmetrical substituted diene wherein at least one of the symmetry planes passes through the nitrogen-nitrogen bonds of at least one tetrazine ring, an unsymmetrical substituted diene, or an isomer-free dienophile,
wherein the reaction kinetics for the inverse electron demand Diels-Alder cycloaddition between the first and second chemical entities has a minimum rate constant of 500 M⁻¹ s⁻¹ in PBS at 25° C., determined by spectrophotometry with the proviso that when the labeling agent in step a) is ⁹⁴Tc, ^{99m}Tc, ²¹¹At, ²²³Ra or ²²⁵Ac the labeling agent is conjugated to an unsymmetrical substituted diene; and
c) oxidizing the ligated labeled targeting vector obtained from step b) at a temperature ranging from 15° C. to 50° C. for up to 60 minutes by adding from 1 to 100 equivalents of a quinone oxidant, such as chloranil, fluoranil, DDQ or NaNO2.

The pharmaceutic agent, imaging agent or therapeutic agent that the first chemical entity is conjugated to is in some embodiments identical with the labeling agent. This may for instance be the case when the labeling agent is an agent that can be applied both as a label and as a therapeutic or imaging agent.

In some embodiments, the labeling agent is a radionuclide. Some radionuclides can be applied both in imaging, in diagnostics and/or in therapy and in the present examples, the same radionuclide have been applied as labeling agent as well as imaging or therapeutic agent. Labeling of a diene or dienophile with a radionuclide will normally not provide 100% labeling efficiency with the radionuclide, some of the products labeled will inevitably be labeled with a stable isotope of the corresponding radionuclide element.

If using a symmetrical substituted diene as a starting point as the entity to be radiolabeled, it will only be possible to provide a radiolabeled single isomeric chemical entity targeting vector if the radiolabel exists in both a radioactive and in a stable form because the symmetrical substituted diene will comprise two targets for the radionuclide/the stable isotope.

Some of the radionuclides that are of interest in therapy and imaging are, however, not obtainable in a stable form. Thus, no corresponding element can label the symmetric position of the labeling target and this would inevitably result in more than one isomeric form of the final product. Accordingly, no symmetric substituted diene can be obtained if the labeling agent is ²¹¹At, ²²³Ra or ²²⁵Ac, and therefore, when these radionuclides are used as label and/or as diagnostic/therapeutic agent ligation combination option ii) should be applied, wherein an unsymmetrical substituted diene is ligated with an isomer-free dienophile. Therefore, the method is to be used with the proviso that when the labeling agent in step a) is ⁹⁴Tc, ^{99m}Tc, ²¹1At, ²²³Ra or ²²⁵Ac the labeling agent is conjugated to an unsymmetrical substituted diene; and The method enables labeling such as radiolabeling, for example with ¹⁸F, radioiodine (¹²³I, ¹²⁴I, ¹²⁵I or ¹³¹I) and ²¹¹At, of any targeting vector in unmatched efficiency and practicality. The ground-breaking nature of the method is the possibility of forming a single end-product, within 60, often within much less than 60 minutes such as within 1-20 minutes, and thereby easing clinical translation. In contrast, conventional tetrazine ligations result in multiple products and as a result need massive and unmanageable toxicological packages or tedious and time-consuming separation.

### Definitions

A symmetrical tetrazine means, in the context of the present invention, any tetrazine that as a “cold” reference or after radioactive labeling/deprotection shows one or more symmetry planes in the chemical structure. One of the symmetry planes passes through the nitrogen-nitrogen bonds of the tetrazine ring(s).

A cold reference means, in the context of the present invention, a compound that is labeled with a non-radioactive isotope of an atom, where a radioactive isotope of the same atom is required in order to provide a radiolabeled version of the same compound. The term cold reference moreover includes, in the context of the present invention a compound that comprises one or more protective group(s) that will be replaced by the labeling agent upon labeling.

Unsymmetrical tetrazine means, in the context of the present invention, any tetrazine that as a “cold” reference or after radioactive labeling/deprotection has no symmetry planes passing through the nitrogen-nitrogen bonds of the tetrazine ring.

Isomer-free dienophile or isomer-free TCH/TCO/TCN means, in the context of the present invention, any dienophile or any TCH, TCO and TCN, respectively, that after reaction and oxidation with a corresponding tetrazine results in the formation of only one isomer/enantiomer.

TCH means, in the context of the present invention, any 7-membered ring with at least one double bond in trans-configuration able to react as a dienophile in an inverse electron demand Diels-Alder cycloaddition.

TCO means, in the context of the present invention, any 8-membered ring with at least one double bond in a trans-configuration able to react as a dienophile in an inverse electron demand Diels-Alder cycloaddition.

TCN means, in the context of the present invention, any 9-membered ring with at least one double bond in a trans-configuration able to react as a dienophile in an inverse electron demand Diels-Alder cycloaddition.

It is well known within click chemistry to use dienes such as tetrazines that are clicked with dienophiles such as trans-cycloheptenes (TCH's), trans-cyclooctene (TCO's) or a trans-cyclononenes (TCN's) with complementary inverse electron demand Diels-Alder cycloaddition reactivity. In a preferred embodiment, the method for providing a labeled single isomeric chemical entity targeting vector the diene is a tetrazine and the dienophile is a trans-cycloheptene (TCH), a trans-cyclooctene (TCO) or a trans-cyclononene (TCN).

The labeled single isomeric chemical entity targeting vectors obtainable by the method according to the present invention, can be applied for various purposes depending on the characteristics of the agent applied as a label. Labeling agents that are suitable for the method includes radiolabels and fluorescent labels.

In a preferred embodiment, the labeling agent applied in step a) in the method for providing a labeled single isomeric chemical entity targeting vector is a radionuclide or a stable isotope of a corresponding element. The characteristics and accordingly the use of the different radionuclides normally applied are well known in the art.

Radionuclide labeling agents and stable isotopes of a corresponding element that are suitable for use as a labeling agent in step a) in the method for providing a labeled single isomeric chemical entity targeting vector includes: ¹H, ²H, ³H, ¹¹C, ¹²C, ¹³C, ¹⁴C ¹³N, ¹⁴N, ¹⁵N ¹⁸F, ¹⁹F, ¹²³I, ¹²⁴I, ¹²⁵I, ¹²⁷I, ¹³¹I, ¹⁵O, ¹⁶O, ¹⁷O, ¹⁸O, ⁴³Sc, ⁴⁴Sc, ⁴⁵Sc, ⁴⁵Ti, ⁴⁶Ti, ⁴⁷Ti, ⁴⁸Ti, ⁴⁹Ti, ⁵⁰Ti, ⁵⁵Co, ^{58m}Co, ⁵⁹Co, ⁶⁰Cu, ⁶¹Cu, ⁶³Cu, ⁶⁴Cu, ⁶⁵Cu, ⁶⁷Cu, ⁶⁷Ga, ⁶⁸Ga, ⁶⁹Ga, ⁷¹Ga, ⁷⁶Br, ⁷⁷Br, ⁷⁹Br, ^{80m}Br, ⁸¹Br, ⁷²As, ⁷⁵As, ⁸⁶Y, ⁸⁹Y, ⁹⁰Y, ⁸⁹Zr, ⁹⁰Zr, ⁹¹Zr, ⁹²Zr, ⁹⁴Zr, ¹⁴⁹Tb, ¹⁵²Tb, ¹⁵⁹Tb, ¹⁶¹Tb, ¹¹¹In, ¹¹³In, ¹¹⁴ mIn, ¹¹⁵mIn, ¹⁷⁵Lu, ¹⁷⁷Lu, ¹⁸⁵Re, ¹⁸⁶Re, ¹⁸⁸Re, ²⁰¹Tl, ²⁰³Tl, ²⁰⁵Tl, ²⁰⁶Pb, ²⁰⁷Pb, ²⁰⁸Pb, ²¹²Pb, ²⁰⁹Bi, ²¹²Bi, ²¹³Bi, ³¹P, ³²P, ³³P, ³²S, ³⁵S ⁴⁵Sc, ⁴⁷Sc, ⁸⁴Sr, ⁸⁶Sr, ⁸⁷Sr, ⁸⁸Sr, ⁸⁹Sr, ¹⁶⁵Ho, ¹⁶⁶Ho, ¹⁵⁶Dy, ¹⁵⁸Dy, ¹⁶⁰Dy, ¹⁶¹Dy, ¹⁶²Dy, ¹⁶³Dy, ¹⁶⁴Dy, ¹⁶⁵Dy, ²²⁷Th, ²³²Th, ⁵¹Cr, ⁵²Cr, ⁵³Cr, ⁵⁴Cr, ⁷³Se, ⁷⁴Se, ⁷⁵Se, ⁷⁶Se, ⁷⁷Se, ⁷⁸Se, ⁸⁰Se ⁸²Se, ⁹⁴Tc, ^{99m}Tc, ¹⁰³Rh, ^{103m}Rh, ¹¹⁹Sb, ¹²¹Sb, ¹²³Sb, ¹³⁵La, ¹³⁸La, ¹³⁹La, ¹⁶²Er, ¹⁶⁴Er, ¹⁶⁵Er ¹⁶⁶Er, ¹⁶⁷Er, ¹⁶⁸Er, ¹⁷⁰Er, ¹⁹³mPt, ¹⁹⁵mPt, ¹⁹²Pt, ¹⁹⁴Pt, ¹⁹⁵Pt, ¹⁹⁶Pt, ¹⁹⁸Pt ²¹¹At, ²²³Ra ²²⁵Ac.

In a preferred embodiment, the radionuclide labeling agents is selected from the group consisting of: ¹¹C, ¹³N, 150, ¹⁸F, ⁴³Sc, ⁴⁴Sc, ⁴⁵Ti, ⁵⁵Co, ⁶⁰Cu, ⁶¹Cu, ⁶⁴Cu, ⁶⁸Ga, ⁷⁶Br, ⁷²As, ⁸⁶Y, ⁸⁹Zr, ⁹⁰Y, ¹⁴⁹Tb, ¹⁵²Tb; and the stable isotopes of the corresponding element is selected from the group consisting of: ¹²C ¹³C, ¹⁴N, ¹⁵N, ¹⁶O, ¹⁷O ¹⁸O, ¹⁹F, ⁴⁵Sc, ⁴⁶Ti, ⁴⁷Ti, ⁴⁸Ti ⁴⁹Ti ⁵⁰Ti, ⁵⁹Co, ⁶³Cu, ⁶⁵Cu, ⁶⁹Ga, ⁷¹Ga, ⁷⁵As, ⁷⁹Br, ⁸¹Br, ⁸⁹Y, ⁹⁰Zr, ⁹¹Zr, ⁹²Zr, ⁹⁴Zr, ¹⁵⁹Tb. These radionuclides and their stable isotopes of the corresponding elements are particularly useful in Positron Emission Tomography (PET).

In another preferred embodiment, the radionuclide labeling agents is selected from the group consisting of: ⁶⁴Cu, ⁶⁷Cu, ⁶⁷Ga, ¹¹¹In, ¹³¹I, ¹⁷⁷Lu, ¹⁸⁶Re, ²⁰¹Ti, ²¹²Pb, ²¹³Bi; and the stable isotope of the corresponding element is selected from the group consisting of: ⁶³Cu, ⁶⁵Cu, ⁶⁹Ga, ⁷¹Ga, ¹¹³In, ¹²⁷I, ¹⁷⁵Lu, ¹⁸⁵Re, ²⁰³Tl, ²⁰⁵Ti, ²⁰⁶Pb, ²⁰⁷Pb, ²⁰⁸Pb, ²⁰⁹Bi. These radionuclides and their stable isotopes of the corresponding elements are particularly useful in Single Photon Emission Computed Tomography (SPECT).

In another preferred embodiment, the radionuclide labeling agents is selected from the group consisting of: ³²P, ³³P, ⁴⁷Sc, ⁶⁴Cu, ⁶⁷Cu, ⁸⁹Sr, ⁹⁰Y, ¹⁶⁶Ho, ¹⁶¹Tb, ¹⁶⁵Dy, ¹⁷⁷Lu, ¹⁸⁶Re, ¹⁸⁸Re; and the stable isotope of the corresponding element is selected from the group consisting of: ³¹p ⁴⁵Sc, ⁶³Cu, ⁶⁵Cu, ⁸⁴Sr, ⁸⁶Sr, ⁸⁷Sr, ⁸⁸Sr, ⁸⁹Y, ¹⁶⁵Ho, ¹⁵⁹Tb, ¹⁵⁶Dy ¹⁵⁸Dy ¹⁶⁰Dy, ¹⁶¹Dy, ¹⁶²Dy ¹⁶³Dy, ¹⁶⁴Dy ¹⁷⁵Lu, ¹⁸⁵Re. These radionuclides are beta-particle emitters and these radionuclides along with their stable isotopes of the corresponding element are applied in therapy for instance in relation to the treatment of various tumorous diseases. In another preferred embodiment, the radionuclide labeling agents is selected from the group consisting of: ¹⁴⁹Tb, ²¹²Pb, ²¹²Bi, ²¹³Bi, ²²⁷Th; and the stable isotope of the corresponding element is selected from the group consisting of: ¹⁵⁹Tb, ²⁰⁶Pb, ²⁰⁷Pb, ²⁰⁸Pb ²⁰⁹Bi, ²³²Th. These radionuclides are alpha-particle emitters and these radionuclides along with their stable isotopes of the corresponding element are applied in therapy for instance in relation to the treatment of various tumorous diseases.

In another preferred embodiment, the radionuclide labeling agents is selected from the group consisting of: ⁵¹Cr, ⁵⁸mCo, ⁶⁴Cu, ⁶⁷Ga, ⁷³Se, ⁷⁵Se, ⁷⁷Br, ^{80m}Br, ⁹⁴Tc, ^{99m}Tc, I, ⁰³mRh, ¹¹¹In, ¹¹⁴mIn, ¹¹⁵mIn, ¹¹⁹Sb, ¹²³I, ¹²⁴I, ¹²⁵I, ¹³⁵La, ¹⁶⁵Er, ¹⁹³mPt, ¹⁹⁵mPt; and the stable isotope of the corresponding element is selected from the group consisting of: ⁵²Cr, ⁵³Cr, ⁵⁴Cr, ⁵⁹Co, ⁶³Cu, ⁶⁵Cu, ⁶⁹Ga, ⁷¹Ga, ⁷⁴Se, ⁷⁶Se, ⁷⁷Se, ⁷⁸Se ⁸⁰Se ⁸²Se, ⁷⁹Br, ⁸¹Br, ¹⁰³Rh ¹¹³In, ¹²¹Sb, ¹²³Sb, ¹²⁷I, ¹³⁸La, ¹³⁹La ¹⁶²Er, ¹⁶⁴Er, ¹⁶⁶Er, ¹⁶⁷Er, ¹⁶⁸Er, ¹⁷⁰Er, ¹⁹²Pt 1, ⁹⁴Pt ¹⁹⁵Pt, ¹⁹⁶Pt, ¹⁹⁸Pt. These radionuclides emit electrons via the Auger effect with low kinetic energy. These radionuclides along with their stable isotopes of the corresponding element are applied in Auger therapy for instance in relation to highly targeted treatment of various tumorous diseases.

In another preferred embodiment, the radionuclide labeling agents is selected from the group consisting of: ³H, ¹⁴C and ³⁵S and the stable isotope of the corresponding element is selected from the group consisting of: ¹H, ²H, ¹²C, ¹³C, ³²S. These radionuclides are applied to in vitro studies such as displacement and tritiation assays.

In another preferred embodiment, the radionuclide labeling agents is selected from the group consisting of: ¹¹C, ¹³N, ¹⁸F, ¹²³I, ¹²⁵I, ¹³¹I, or ²¹¹At; and the stable isotope of the corresponding element (when such a stable isotope of the element is obtainable) is selected from the group consisting of: ¹²C, ¹⁴N, ¹⁹F, ¹²⁷I. These radionuclides along with their stable isotopes of the corresponding element if available, are among the most frequently used radionuclides in therapy and imaging presently.

The targeting vector that is conjugated to either the diene or to the dienophile mentioned in step b) in the for providing a labeled single isomeric chemical entity targeting vector can be any kind of targeting vector that is suitable for use in therapy, imaging, or diagnostics. Such commonly used targeting vectors that are suitable in the present method include antibodies, nanobodies, polymers, nanomedicines, cells, proteins, peptides, and small molecules.

Commonly applied targeting vectors, that are suitable in the present method includes: peptides such as Octreotide, Octreotate, AE105; small molecules such as FAPI derivatives and PSMA derivatives.

In a preferred embodiment, the targeting vector applied in the method for providing a labeled single isomeric chemical entity targeting vector is selected from the group comprising: Octreotide, Octreotate, AE105, FAPI derivatives and PSMA derivatives. The oxidizing step c) in the method for providing a labeled single isomeric chemical entity targeting vector is carried out at a certain temperature and time, by adding a specific oxidant to the ligated compound obtained in step b). These conditions ensures that the efficiency of the oxidation step is ≥90% thereby meeting the speed required for therapeutic, diagnostic or imaging use of the labeled single isomeric chemical entity targeting vector.

The time required to obtain an oxidation efficiency of ≥90% depends on the specific compound being oxidized, temperature, oxidant equivalents and on the oxidation agent. With the conditions applied in the present method, the ≥90% oxidation efficiency will be obtained within 60 minutes, such as from 0-50 minutes, from 0-40 minutes, from 0-30 minutes, from 0-20 minutes, from 0-10 minutes, or from 0-5 minutes. In a preferred embodiment, the oxidation efficiency obtained is ≥90% in 0-20 minutes.

The temperature for the oxidation step is 15° C.-50° C., such as 15° C.-45° C., 15° C.-40° C., 15° C.-35° C., 20° C.-30° C., or at approximately 20° C.-25° C. The preferred temperature is room temperature such as between 20° C.-25° C.

The oxidant should be a quinone oxidant, selected from chloranil, fluoranil, DDQ, or NaNO₂. It has surprisingly been found herein, that using other types of oxidants will not provide the desired single isomeric form of the labeled chemical entity targeting vector or will negatively impact the structure of the targeting vector. The oxidant is added to the ligated labeled compound obtained from step b) in the method for providing a labeled single isomeric chemical entity targeting vector from 1 to 100 equivalents of the product obtained in step b), such as from 10 to 90, 20 to 80, 30 to 70, 40 to 60 or 50 equivalents of the product obtained in step b). Preferably 1 to 10, such as 1 equivalent oxidant is added to the labeled compound obtained from step b).

In one embodiment, the oxidant is solid phase supported. Any commonly available solid support would be applicable, for instance oxidants supported by alumina, silica gel, polymer, montmorillonite, zeolite or a nanomaterial. The advantages of using a solid supported oxidants in general include easy removal from reactions by filtration, excess reagents can be used to drive reactions to completion without introducing difficulties in purification, easy to handle, recycling of recovered reagents is economical, and efficient.

The conventional ligation between a tetrazine and a TCO will result in a number of different tautomers, regioisomers and enantiomers as schematically shown in Scheme 1.

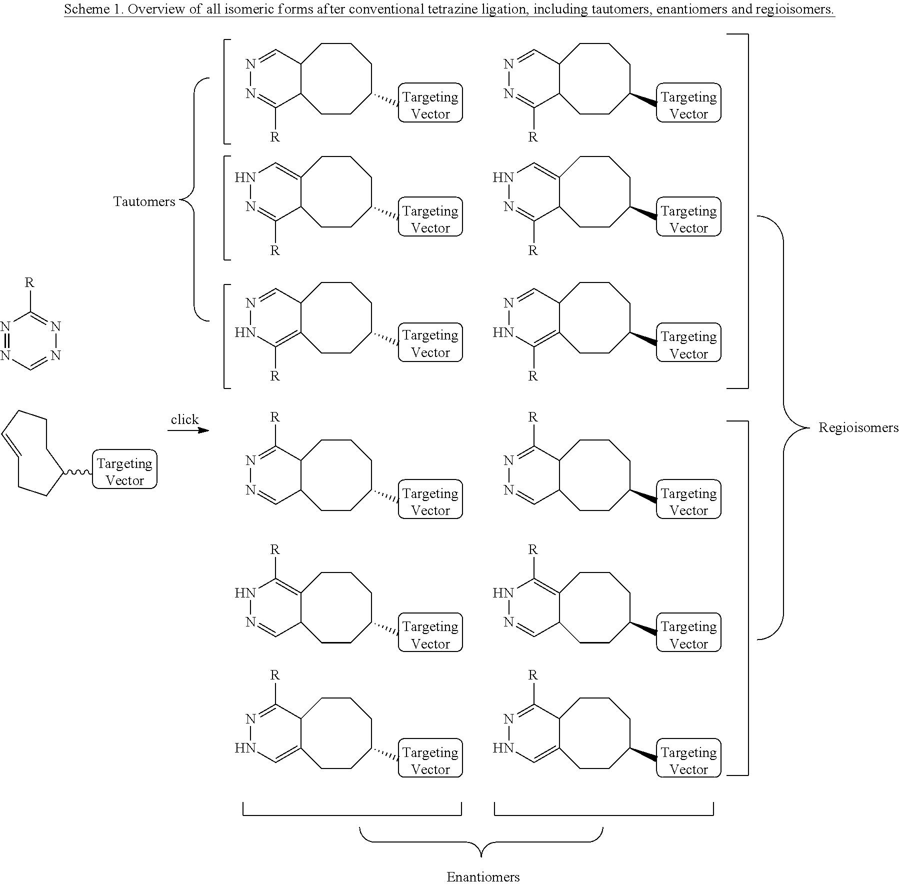

The below scheme 2 is an example of a current state-of-the-art tetrazine ligation. A reaction which gives too many isomeric products:

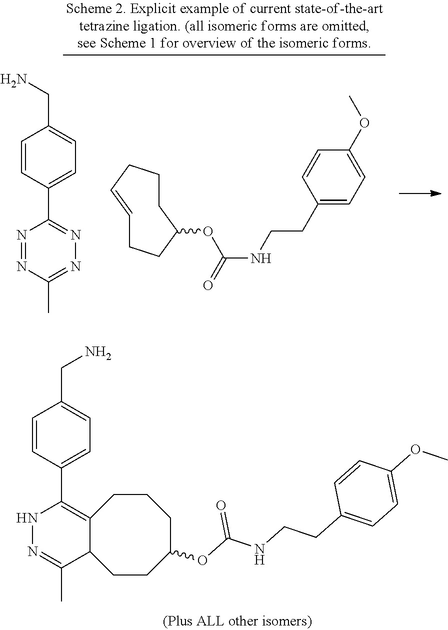

Multiple isomers (at least 12 isomers) can be formed in this reaction, which all have very similar polarities and are accordingly very difficult to separate for instance by HPLC.

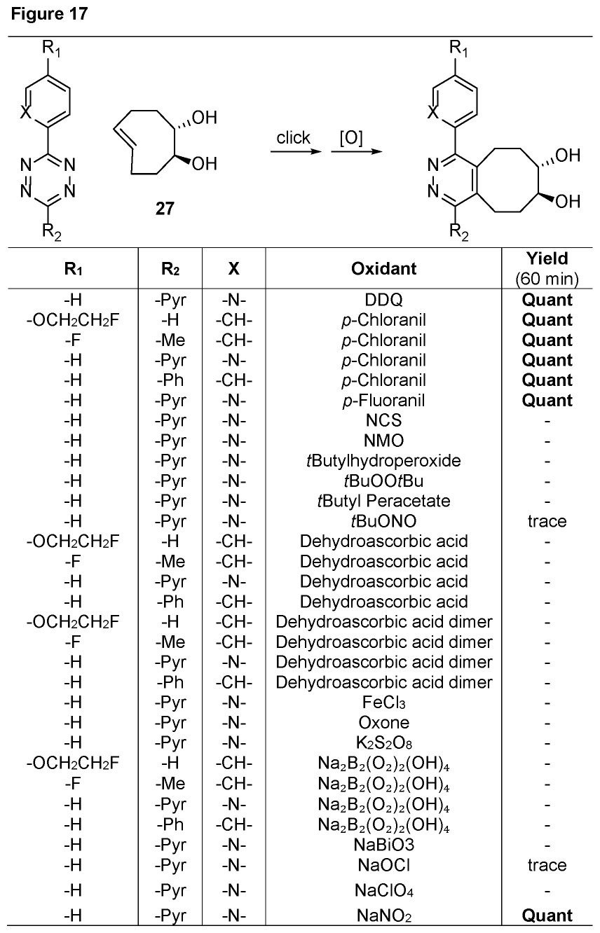

Herein, we describe a method, which allows for the use of a labeled first chemical entity such as radiolabelled tetrazine-based synthons in the radiolabelling of isomer-free dienophiles, such as isomer-free trans-cycloheptenes (TCH), isomer-free trans-cyclooctene (TCO) and isomer-free trans-cyclononene (TCN) functionalized vectors and vice versa, which upon subsequent chemical oxidation yields a single final compound within short time, such as within 0-60 minutes (FIG. 17). The method comprises two steps: a ligation step followed by an oxidation step.

In a preferred embodiment, the method for providing a labeled single isomeric chemical entity targeting vector comprises:
a) labeling a first chemical entity having inverse electron demand Diels-Alder cycloaddition reactivity and being conjugated to a pharmaceutic agent, an imaging agent, or a therapeutic agent, with a labeling agent; wherein the first chemical entity is selected from the group consisting of a symmetrical tetrazine wherein at least one of the symmetry planes passes through the nitrogen-nitrogen bonds of at least one tetrazine ring, an unsymmetrical tetrazine, an isomer-free trans-cycloheptene (TCH), an isomer-free trans-cyclooctene (TCO), and an isomer-free trans-cyclononene (TCN),
b) ligating the labeled first chemical entity obtained in step a) with a second chemical entity having complementary inverse electron demand Diels-Alder cycloaddition reactivity and being conjugated to a targeting vector; wherein the second chemical entity is selected from the group consisting of a symmetrical tetrazine wherein at least one of the symmetry planes passes through the nitrogen-nitrogen bonds of at least one tetrazine ring, an unsymmetrical tetrazine, an isomer-free trans-cycloheptene (TCH), an isomer-free trans-cyclooctene (TCO), an isomer-free trans-cyclononene (TCN), and a pre-meso trans-cycloheptene (TCH),
   wherein the reaction kinetics for the inverse electron demand Diels-Alder cycloaddition between the first and second chemical entities has a minimum rate constant of 500 M⁻¹ s⁻¹ in PBS at 25° C., determined by stopped-flow spectrophotometry,
      with the proviso that when the labeling agent in step a) is ⁹⁴Tc, ^{99m}Tc, ²¹¹At, ²²³Ra or ²²⁵Ac the labeling agent is conjugated to an unsymmetrical substituted diene; and c) oxidizing the ligated labeled targeting vector obtained from step b) at a temperature ranging from 20° C. to 30° C. for up to 20 minutes by adding from 1 to 100 equivalents of chloranil, fluoranil, DDQ or NaNO₂.

The starting entities to be ligated is an isomer-free dienophile such as an isomer-free TCH, TCO, or TCN and a diene such as a tetrazine. The diene can either be an unsymmetrical substituted diene, such as an unsymmetrical substituted tetrazine, or a symmetrical substituted tetrazine wherein at least one of the symmetry planes passes through the nitrogen-nitrogen bonds of at least one tetrazine ring, such as a symmetrical tetrazine. Using an isomer-free dienophile such as an isomer-free TCH, TCO, or TCN, reduces the number of formed click-products, by eliminating all enantiomeric and regioisomeric products. The formed tautomeric entities such as dihydropyridazines will be subsequently oxidized to the corresponding single isomeric form such as a pyridazine, resulting in a single product. The ‘R’ substituents on the diene, such as a tetrazine, employed in this method will typically be functionalized with an aryl substituted with ¹⁸F, ¹²³I, ¹²⁴I, ¹²⁵I, ¹³¹I or ²¹¹At.

The below scheme 2 and scheme 3 are illustrations of examples of ligations in accordance with the method of the invention here exemplified in using an unsymmetrical tetrazine and a TCO conjugated to a targeting vector:

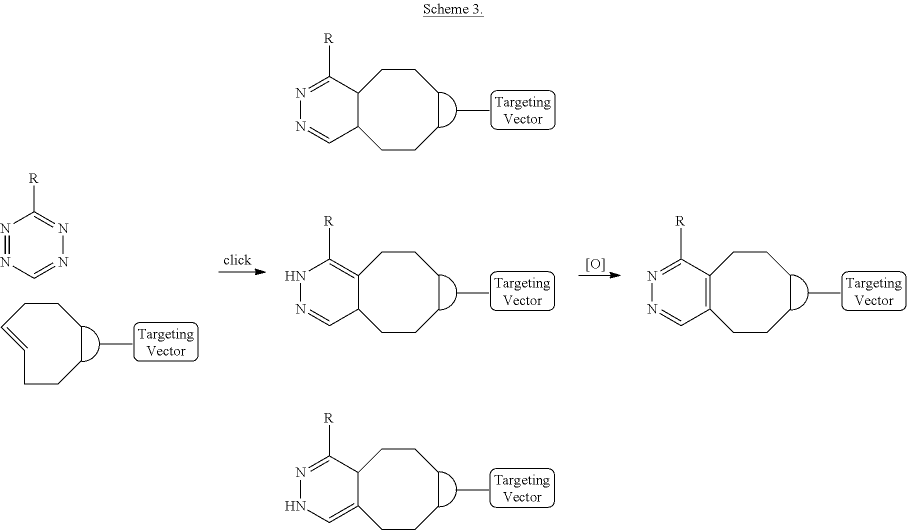

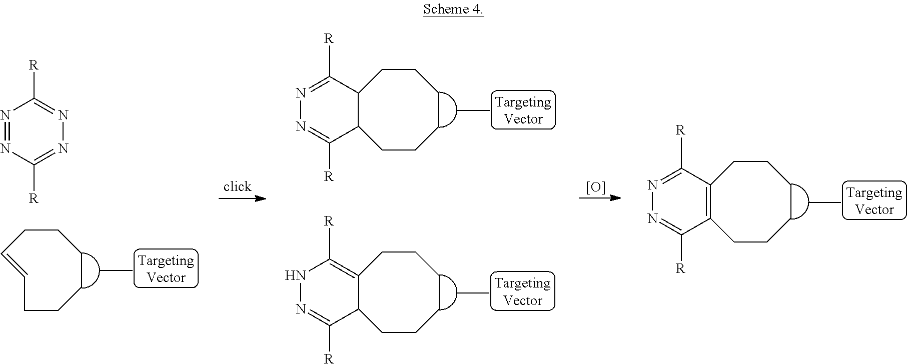

The targeting vector is an antibody, a nanobody, a polymer, a nanomedicine, a cell, a protein, a peptide, or a small molecule.

When the labeling agent in step a) is ²¹At, ²²³Ra or ²²⁵Ac the labeling agent must be conjugated to the unsymmetrical substituted diene.

The following dienes are examples for symmetrical tetrazines of formula Tz1 suitable for ligation in step b) of the method for providing a labeled single isomeric chemical entity targeting vector:

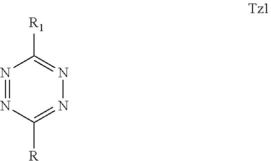

wherein R and R₁ are

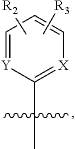

wherein the curly sign indicates the link to the tetrazine; and where R₂ is —H or (i) an isotope labeling agent directly connected to the aromatic ring; or (ii) an isotope labeling agent connected to the aromatic ring via a linker, said linker being selected from the group consisting of (CH₂)ₙ, -LO(CH₂)ₙ, -LNH(CH₂)ₙ, -LCONH(CH₂)ₙ, -LNHCO(CH₂)ₙ, where L is —(CH₂)ₘ or —O(CH₂CH₂O)ₘ, where n and m are independently selected from 1-25; or (iii) an isotope labeling agent that is chelated through a chelator selected from: 1,4,7,10-tetraazacyclododecane-N,N′,N′,N″-tetraacetic acid (DOTA), N,N′bis(2-hydroxy-5-(carboxyethyl)benzyl)ethylenediamine N,N′-diacetic acid (HBED-CC), 14,7-triazacyclononane-1,4,7-triacetic acid (NOTA), 2-(4.7-bis(carboxymethyl)-1,4,7-triazonan-1-yl)pentanedioic acid (NODAGA), 2-(4,7,10-tris(carboxymethyl)-1,4,7,10-tetraazacyclododecan-1-yl)pentanedioic acid (DOTAGA), 14,7-triazacyclononane phosphinic acid (TRAP), 14,7-triazacyclononane-1-methyl(2-carboxyethyl)phosphinic acid-4,7-bis(methyl(2-hydroxymethyl)phosphinic acid (NOPO), 3,6,9,15-tetraazabicyclo9.3.1.pentadeca-1 (15),11,13-triene-3,6,9-triacetic acid (PCTA), N-(5-acetyl (hydroxy)aminopentyl-N-(5-(4-(5-aminopentyl)(hydroxy)amino-4-oxobutanoyl)amino)pentyl-N-hydroxysuccinamide (DFO), diethylenetriaminepentaacetic acid (DTPA), trans-cyclohexyl-diethylenetriaminepentaacetic acid (CHX-DTPA), 1-oxa-4,7,10-triazacyclododecane-4,7,10-triacetic acid (OXO-Do3A), p-isothiocyanatobenzyl-DTPA (SCN-BZ-DTPA), 1-(p-isothiocyanatobenzyl)-3-methyl-DTPA (1B3M), 2-(p-isothiocyanatobenzyl)-4-methyl-DTPA (1M3B), and 1-(2)-methyl-4-isocyanatobenzyl-DTPA (MX-DTPA) connected to the aromatic ring through a linker, said linker being selected from the group consisting of (CH₂)ₙ, -LO(CH₂)ₙ, -LNH(CH₂)ₙ, -LCONH(CH₂)ₙ, -LNHCO(CH₂)ₙ, where L is —(CH₂)ₘ or —O(CH₂CH₂O)ₘ, and n and m are independently selected from 1-25;
wherein, when R₂ is either (i) or (ii) the isotope labeling agent is selected from the group consisting of:
¹H, ²H, ³H, ¹¹C, ¹²C, ¹³C ¹³N, ¹⁴N, ¹⁵N ¹⁸F, ¹⁹F, ¹²³I, ¹²⁴I, ¹²⁵I, ¹²⁷I, ¹³¹I, ²¹¹At, ¹⁵O, ¹⁶O, ¹⁷O, ¹⁸O, ³²S, ³⁵S, ⁴³Sc, ⁴⁴Sc, ⁴⁵Sc, ⁴⁵Ti, ⁴⁶Ti, ⁴⁷Ti, ⁴⁸Ti, ⁴⁹Ti, ⁵⁰Ti, ⁵⁵Co, ⁵⁸mCo, ⁵⁹Co, ⁶⁰Cu, ⁶¹Cu, ⁶³Cu, ⁶⁴Cu, ⁶⁵Cu, ⁶⁷Cu, ⁶⁷Ga, ⁶⁸Ga, ⁶⁹Ga, ⁷¹Ga, ⁷⁶Br, ⁷⁷Br, ⁷⁹Br, ^{80m}Br, ⁸¹Br, ⁷²As, ⁷⁵As, ⁸⁶Y, ⁸⁹Y, ⁹⁰Y, ⁸⁹Zr, ⁹⁰Zr, ⁹¹Zr, ⁹²Zr, ⁹⁴Zr, ¹⁴⁹Tb, ¹⁵²Tb, ¹⁵⁹Tb, ¹⁶¹Tb, ¹¹¹In, ¹¹³In, ¹¹⁴mIn, ¹¹⁵ min, ¹⁷⁵Lu, ¹⁷⁷Lu ¹⁸⁵Re, ¹⁸⁶Re, ¹⁸⁸Re, ²⁰¹Tl, ²⁰³Tl, ²⁰⁵Tl, ²⁰⁶Pb, ²⁰⁷Pb, ²⁰⁸Pb, ²¹²Pb, ²⁰⁹Bi, ²¹²Bi, ²¹³Bi, ³¹P, ³²P, ³³P, ⁴⁵Sc, ⁴⁷Sc, ³⁴Sr, ⁸⁶Sr, ⁸⁷Sr, ⁸⁸Sr, ⁸⁹Sr, ¹⁶⁵Ho, ¹⁶⁶Ho, ¹⁵⁶Dy, ¹⁵⁸Dy ¹⁶⁰Dy, ¹⁶¹Dy ¹⁶²Dy, ¹⁶³Dy ¹⁶⁴Dy, ¹⁶⁵Dy ²²⁷Th, ²³²Th, ⁵¹Cr, ⁵²Cr, ⁵³Cr, ⁵⁴Cr, ⁷³Se, ⁷⁴Se, ⁷⁵Se, ⁷⁶Se, ⁷⁷Se, ⁷⁸Se, ⁸⁰Se ⁸²Se, ⁹⁴Tc, ^{99m}Tc ¹⁰³Rh, 1, ⁰³mRh, ¹19Sb, ¹²¹Sb, ¹²³Sb, ¹³⁵La, ¹³⁸La, ¹³⁹La, ¹⁶²Er, ¹⁶⁴Er, ¹⁶⁵Er, ¹⁶⁶Er, ¹⁶⁷Er, ¹⁶⁸Er, ¹⁷⁰Er, ¹⁹³mPt, ¹⁹⁵mPt, ¹⁹²Pt, ¹⁹⁴Pt ¹⁹⁵Pt ¹⁹⁶Pt ¹⁹⁸Pt,
and wherein X and Y are independently selected from: —CH— and —N—;
and wherein R₃ is independently selected from H or a moiety selected from the group consisting of a hydroxy group, a sulfonamide, a carboxyl group, a sulfonyl group, amine, a substituted amine with 1-5 polyethylene glycol unit(s), a —(O—CH₂—CH₂)₁₋₅—OCH₂—COOH, Methyl, Ethyl, Propyl, optionally substituted heteroaryl, and optionally substituted arylalkyl; wherein optionally substituted in relation to said substituted amine means one or more substituents selected from, a halogen, a hydroxy group, a sulfonamide, a carboxyl group, a sulfonyl group, amine, (C1-C10) alkyl, (C2-C10)alkenyl, (C2-C10)alkynyl, (C1-C10)alkylene, (C1-C10)alkoxy, (C2-C10)dialkylamino, (C1-C10)alkylthio, (C2-C10)heteroalkyl, (C2-C10)heteroalkylene, (C3-30 C10)cycloalkyl, (C3-C10)heterocycloalkyl, (C3-10)cycloalkylene, (C3-C10)heterocycloalkylene, (C1-C10)haloalkyl, (C1-C10)perhaloalkyl, (C2-C10)-alkenyloxy, (C3-C10)-alkynyloxy, aryloxy, arylalkyloxy, heteroaryloxy, heteroarylalkyloxy, (C1-C6)alkyloxy-(C1-C4)alkyl, optionally substituted aryl, optionally substituted heteroaryl, and optionally substituted arylalkyl; wherein optionally substituted means one or more substituents selected from a halogen, a hydroxy group, a sulfonamide, a carboxyl group, a sulfonyl group, amine, a substituted amine with 1-5 polyethylene glycol unit(s), a —(O—CH₂—CH₂)₁₋₅—OCH₂—COOH, H, Methyl, Ethyl, Propyl, optionally substituted heteroaryl, and optionally substituted arylalkyl; wherein optionally substituted in relation to said substituted amine means one or more substituents selected from a halogen, a hydroxy group, a sulfonamide, a carboxyl group, a sulfonyl group, and amine;
and wherein R and R₁ are identical or differs only in the isotope number of the labelling agent.

The below tetrazines are preferred tetrazines of Formula Tz1 for use in the ligation in step b) of the method for providing a labeled single isomeric chemical entity targeting vector:

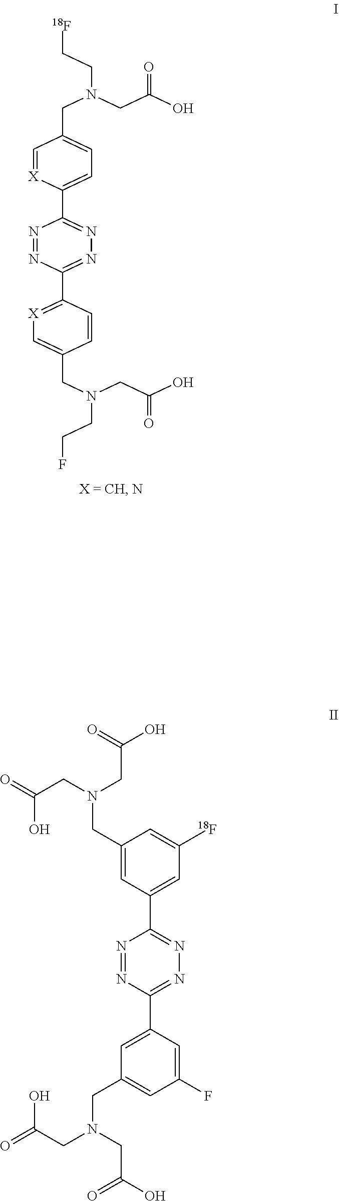

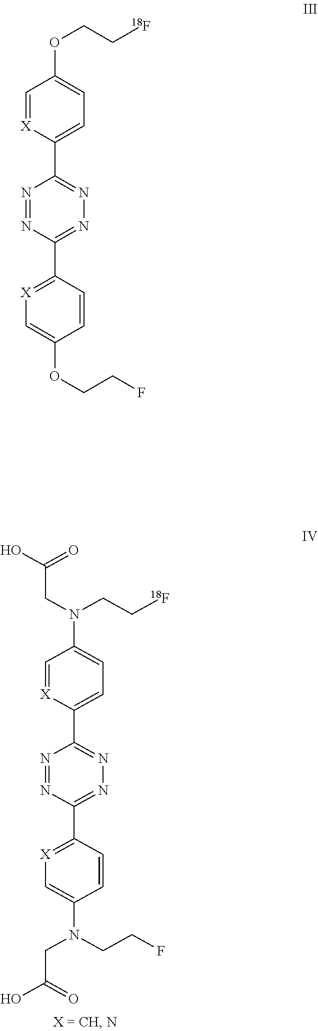

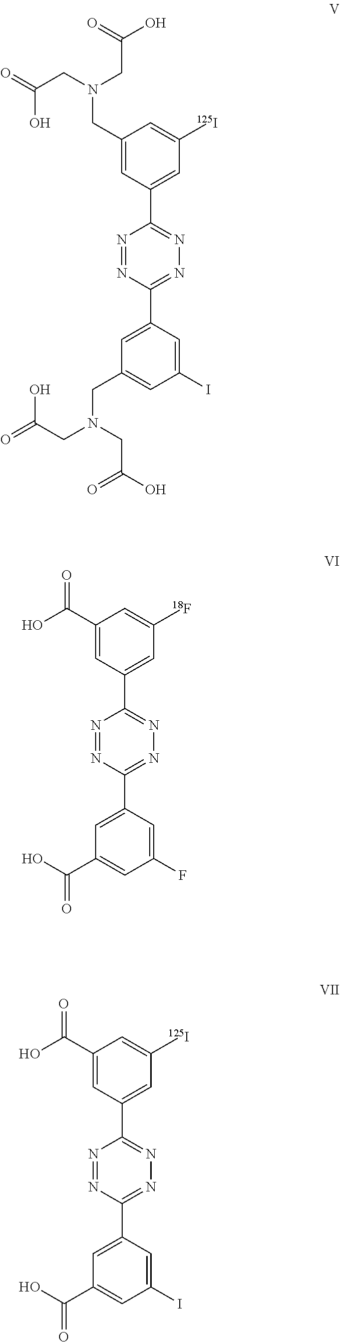 [SMILES: O=C(O)CN(CC(=O)O)Cc1cc(I)cc(-c2nnc(-c3cc([125I])cc(CN(CC(=O)O)CC(=O)O)c3)nn2)c1 ; O=C(O)c1cc(F)cc(-c2nnc(-c3cc([18F])cc(C(=O)O)c3)nn2)c1 ; O=C(O)c1cc(I)cc(-c2nnc(-c3cc([125I])cc(C(=O)O)c3)nn2)c1]

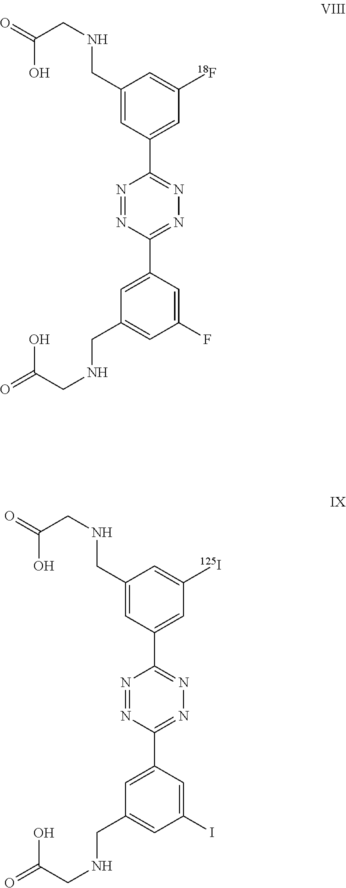 [SMILES: O=C(O)CNCc1cc(F)cc(-c2nnc(-c3cc([18F])cc(CNCC(=O)O)c3)nn2)c1 ; O=C(O)CNCc1cc(I)cc(-c2nnc(-c3cc([125I])cc(CNCC(=O)O)c3)nn2)c1]

Unsymmetrical tetrazines of formula Tz2 are moreover examples of preferred dienes suitable for ligation in step b) of the method for providing a labeled single isomeric chemical entity targeting vector:

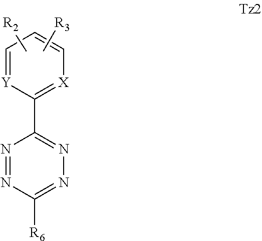

wherein R₄ is —H or (i) an isotope labeling agent directly connected to the aromatic ring or (ii) an isotope labeling agent connected to the aromatic ring via a linker, said linker being selected from the group consisting of (CH₂)ₙ, -LO(CH₂)ₙ, -LNH(CH₂)ₙ, -LCONH(CH₂)ₙ, -LNHCO(CH₂)ₙ, where L is —(CH₂)ₘ or —O(CH₂CH₂O)ₘ, where n and m are independently selected from 1-25, or (iii) an isotope labeling agent that is chelated through a chelator selected from: 1,4,7,10-tetraazacyclododecane-N,N′,N′,N″-tetraacetic acid (DOTA), N,N′-bis(2-hydroxy-5-(carboxyethyl)benzyl)ethylenediamine N,N′-diacetic acid (HBED-CC), 14,7-triazacyclononane-1,4,7-triaceticacid (NOTA), 2-(4.7-bis(carboxymethyl)-1,4,7-triazonan-1-yl)pentanedioic acid (NODAGA), 2-(4,7,10-tris(carboxymethyl)-1,4,7,10-tetraazacyclododecan-1-yl)pentanedioic acid (DOTAGA), 14,7-triazacyclononane phosphinic acid (TRAP), 14,7-triazacyclononane-1-methyl(2-carboxyethyl)phosphinic acid-4,7-bis(methyl(2-hydroxymethyl)phosphinic acid (NOPO), 3,6,9,15-tetraazabicyclo9.3.1.pentadeca-1 (15),11,13-triene-3,6,9-triacetic acid (PCTA), N′-(5-acetyl (hydroxy)aminopentyl-N-(5-(4-(5-aminopentyl)(hydroxy)amino-4-oxobutanoyl)amino)pentyl-N-hydroxysuccinamide (DFO), diethylenetriaminepentaacetic acid (DTPA), trans-cyclohexyl-diethylenetriaminepentaacetic acid (CHX-DTPA), 1-oxa-4,7,10-triazacyclododecane-4,7,10-triacetic acid (OXO-Do3A), p-isothiocyanatobenzyl-DTPA (SCN-BZ-DTPA), 1-(p-isothiocyanatobenzyl)-3-methyl-DTPA (1B3M), 2-(p-isothiocyanatobenzyl)-4-methyl-DTPA (1M3B), and 1-(2)-methyl-4-isocyanatobenzyl-DTPA (MX-DTPA) and connected to the aromatic ring through a linker, said linker being selected from the group consisting of (CH₂)ₙ, -LO(CH₂)ₙ, -LNH(CH₂)ₙ, -LCONH(CH₂)ₙ, -LNHCO(CH₂)ₙ, where L is —(CH₂)ₘ or —O(CH₂CH₂O)ₘ, and n and m are independently selected from 1-25; wherein when R₄ is (i), (ii) or (iii) the isotope labeling agent is selected from the group consisting of: ¹H, ²H, ³H, ¹¹C ¹²C, ¹³C, ¹³N, ¹⁴N, ¹⁵N, ¹⁸F, ¹⁹F, ¹²³I, ¹²⁴I, ¹²⁵I, ¹²⁷I, ¹³¹I, ²¹¹At, ¹⁵O, ¹⁶O, ¹⁷O, ¹⁸O, ³²S, ³⁵S, ⁴³Sc, ⁴⁴Sc, ⁴⁵Sc, ⁴⁵Ti, ⁴⁶Ti ⁴⁷Ti, ⁴⁸Ti ⁴⁹Ti 5i ⁵⁵Co, ⁵⁸mCo, ⁵⁹Co, ⁶⁰Cu, ⁶¹Cu, ⁶³Cu, ⁶⁴Cu, ⁶⁵Cu, ⁶⁷Cu, ⁶⁷Ga, ⁶⁸Ga, ⁶⁹Ga, ⁷¹Ga, ⁷⁶Br, ⁷⁷Br, ⁷⁹Br, ^{80m}Br, ⁸¹Br, ⁷²As, ⁷⁵As, ⁸⁶Y, ⁸⁹Y, ⁹⁰Y, ⁸⁹Zr, ⁹⁰Zr, ⁹¹Zr, ⁹²Zr, ⁹⁴Zr, ¹⁴⁹Tb, ¹⁵²Tb, ¹⁵⁹Tb, ¹⁶¹Tb, ¹¹¹In, ¹¹³In, ¹¹⁴mIn, ¹¹⁵ min, ¹⁷⁵Lu, ¹⁷⁷Lu, ¹⁸⁵Re, ¹⁸⁶Re, ¹⁸⁸Re, ²⁰¹Tl, ²⁰³Tl, ²⁰⁵Tl, ²⁰⁶Pb, ²⁰⁷Pb, ²⁰⁸Pb, ²¹²Pb, ²⁰⁹Bi, ²¹²Bi, ²¹³Bi, ³¹P, ³²P, ³³P ⁴⁵Sc, ⁴⁷Sc, ⁸⁴Sr, ⁸⁶Sr, ⁸⁷Sr, ⁸⁸Sr, ⁸⁹Sr, ¹⁶⁵Ho, ¹⁶⁶Ho, ¹⁵⁶Dy, ¹⁵⁸Dy, ¹⁶⁰Dy, ¹⁶¹Dy, ¹⁶²Dy, ¹⁶³Dy, ¹⁶⁴Dy, ¹⁶⁵Dy, ²²⁷Th, ²³²Th, ⁵¹Cr, ⁵²Cr, ⁵³Cr, ⁵⁴Cr, ⁷³Se ⁷⁴Se ⁷⁵Se ⁷⁶Se ⁷⁷Se ⁷⁸Se ⁸⁰Se ⁸²Se ⁹⁴Tc, ^{99m}Tc ¹⁰³Rh, ¹⁰³mRh, ¹¹⁹Sb, ¹²¹Sb, ¹²³Sb, ¹³⁵La, ¹³⁸La ¹³⁹La, ¹⁶²Er, ¹⁶⁴Er, ¹⁶⁵Er, ¹⁶⁶Er, ¹⁶⁷Er, ¹⁶⁸Er, ¹⁷⁰Er, ¹⁹³mPt, ¹⁹⁵mPt, ¹⁹²Pt, ¹⁹⁴Pt, ¹⁹⁵Pt, ¹⁹⁶Pt, ¹⁹⁸Pt, ²¹¹At, ²²³Ra, ²²⁵Ac,
and wherein X and Y are independently selected from: —CH— and —N—;
and wherein R₆ is H or

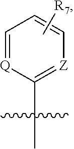

wherein Q and Z are independently selected from: —CH— and —N— and wherein the curly sign indicates the link to the tetrazine; and wherein R₅ and R₇ are independently selected from H or a moiety selected from the group consisting of a hydroxy group, a sulfonamide, a carboxyl group, a sulfonyl group, amine, a substituted amine with 1-5 polyethylene glycol unit(s), a —(O—CH₂—CH₂)₁₋₅—OCH₂—COOH, methyl, ethyl, propyl, optionally substituted heteroaryl, and optionally substituted arylalkyl; wherein optionally substituted in relation to said substituted amine means one or more substituents selected from a halogen, a hydroxy group, a sulfonamide, a carboxyl group, a sulfonyl group, amine, (C1-C10) alkyl, (C2-C10)alkenyl, (C2-C10)alkynyl, (C1-C10)alkylene, (C1-C10)alkoxy, (C2-C10)dialkylamino, (C1-C10)alkylthio, (C2-C10)heteroalkyl, (C2-C10)heteroalkylene, (C3-30 C10)cycloalkyl, (C3-C10)heterocycloalkyl, (C3-10)cycloalkylene, (C3-C10)heterocycloalkylene, (C1-C10)haloalkyl, (C1-C10)perhaloalkyl, (C2-C10)-alkenyloxy, (C3-C10)-alkynyloxy, aryloxy, arylalkyloxy, heteroaryloxy, heteroarylalkyloxy, (C1-C6)alkyloxy-(C1-C4)alkyl, optionally substituted aryl, optionally substituted heteroaryl, and optionally substituted arylalkyl; wherein optionally substituted means one or more substituents selected from a halogen, a hydroxy group, a sulfonamide, a carboxyl group, a sulfonyl group, amine, a substituted amine with 1-5 polyethylene glycol unit(s), a —(O—CH₂—CH₂)₁₋₅—OCH₂—COOH, H, Methyl, Ethyl, Propyl, optionally substituted heteroaryl, and optionally substituted arylalkyl; wherein optionally substituted in relation to said substituted amine means one or more substituents selected from a halogen, a hydroxy group, a sulfonamide, a carboxyl group, a sulfonyl group, and an amine.

The below tetrazines are preferred tetrazines of Formula Tz2 for use in ligation in step b) of the method for providing a labeled single isomeric chemical entity targeting vector:

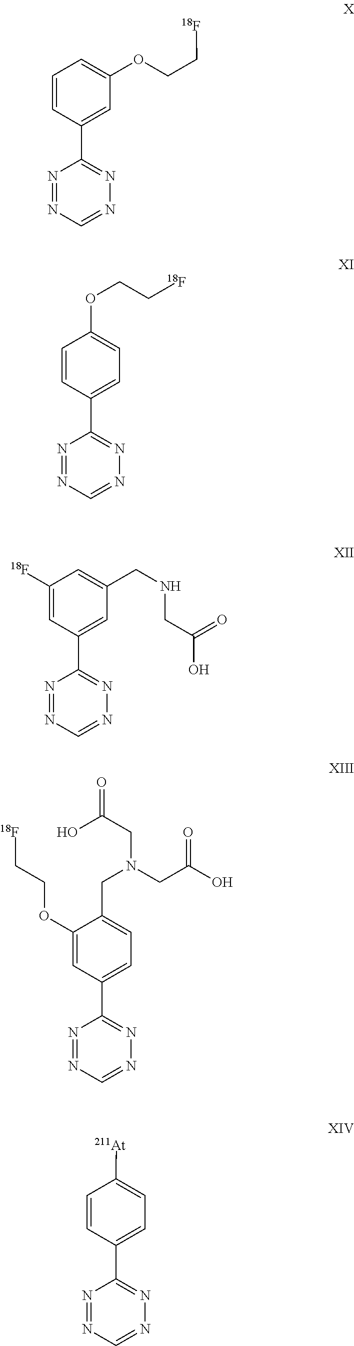

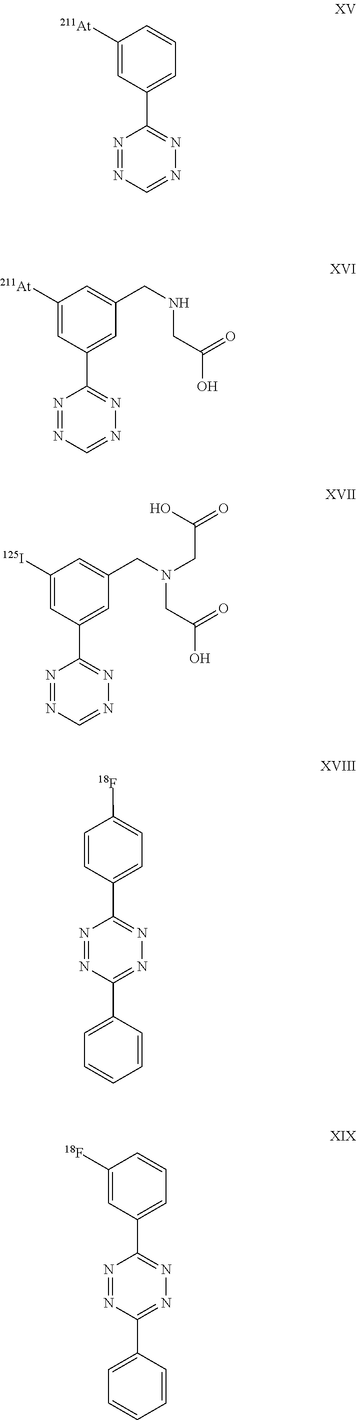

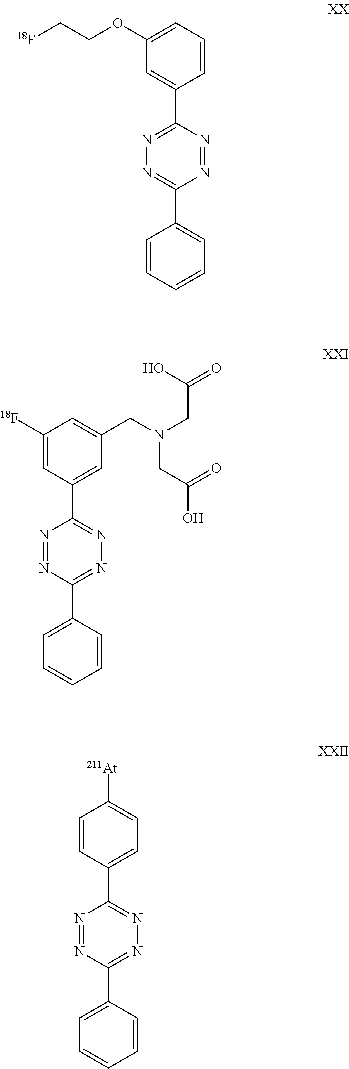

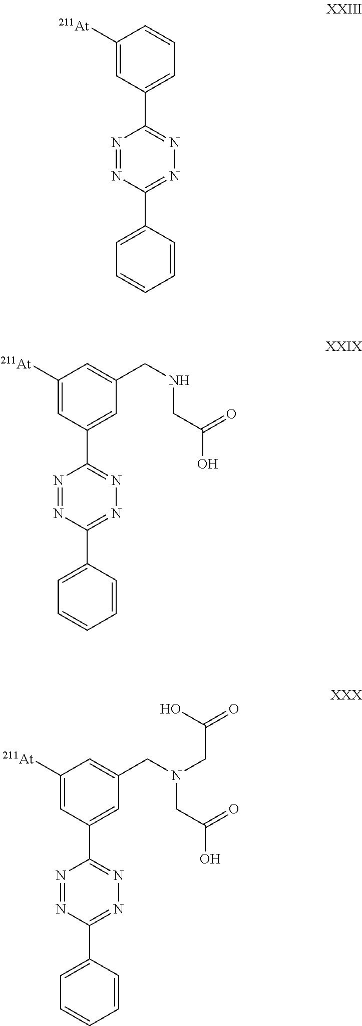

The below trans-cycloheptenes (TCH's), trans-cyclooctenes (TCO's), and a trans-cyclononenes (TCNs) are preferred isomer-free dienophiles for use in ligating in step b) of the method for providing a labeled single isomeric chemical entity targeting vector:

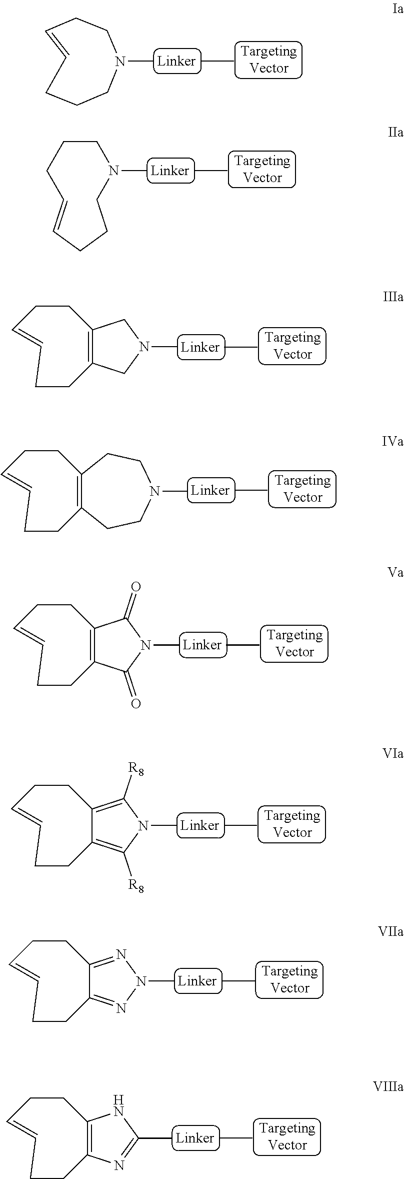

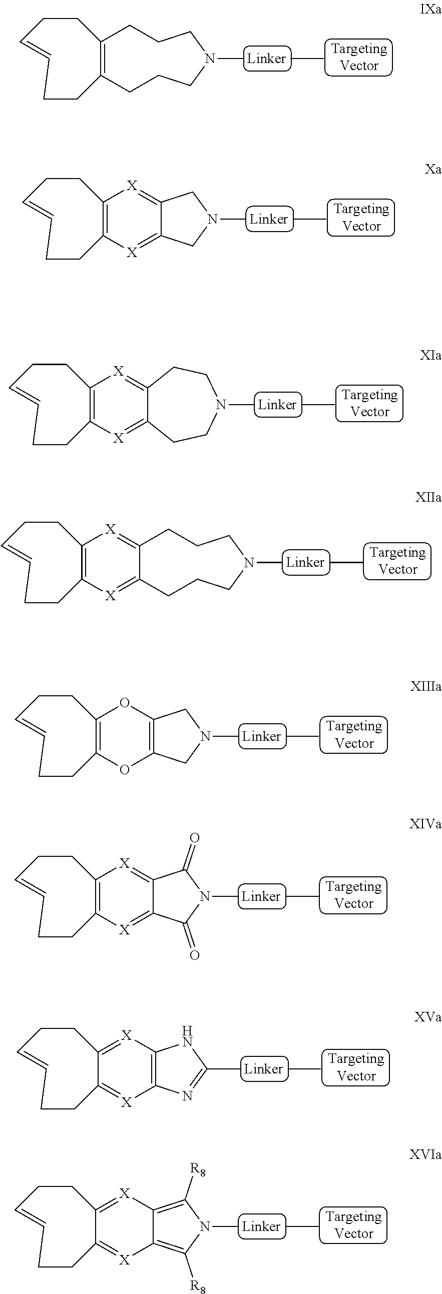

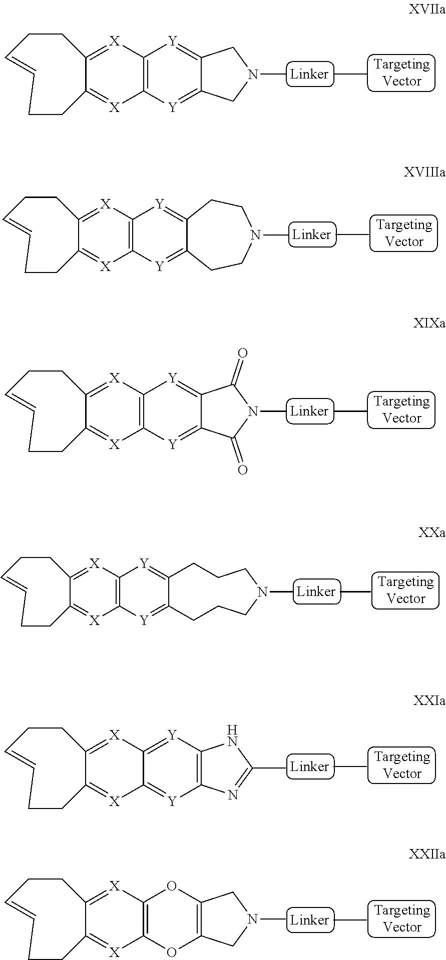

Wherein X is N, NO or CR₃; Y is N, NO or CR₃; R₃ is selected from the group consisting of: —H, —F, —OH, —NH₂, —COOH, —COOCH₃, CF₃, —Cl, —CONH₂, CONHCH₃, —CON(CH₃)₂, —CH₂CH₂OH, —CH₂CH₂NH₂, —CHCH₂N(CH₃)₂ and
wherein the linker is selected from the group comprising: —(CH₂)ₙ— (CH₂)ₙNH, (CH₂)ₙCO, (CH₂)ₙO, (CH₂CH₂O)ₙ, (CH₂CH₂O)ₙCH₂CH₂NH, (CH₂CH₂O)ₙCH₂CH₂CO, —CO(CH)₂— CO(CH₂)ₙNH, CO(CH₂)ₙCO, CO(CH₂)ₙO, CO(CH₂CH₂O)ₙ CO(CH₂CH₂O)ₙCH₂CH₂NH, CO(CH₂CH₂O)ₙCH₂CH₂CO, COO(CH)₂— COO(CH₂)ₙNH, COO(CH₂)ₙCO, COO(CH₂)ₙO, COO(CH₂CH₂O)ₙ COO(CH₂CH₂O)ₙCH₂CH₂NH, COO(CH₂CH₂O)ₙCH₂CH₂CO, CONH(CH)₂—CONH(CH₂)ₙNH, CONH(CH₂)ₙCO, CONH(CH₂)ₙO, CONH(CH₂CH₂O)ₙ, CONH(CH₂CH₂O)ₙCH₂CH₂NH, CONH(CH₂CH₂O)ₙCH₂CH₂CO, —CONHPhCO, —COOPhCO, —COPhCO, CONHCHMCO, (CH₂)ₙNHCHMCO, (CH₂)ₙOCONHCHMCO, (CH₂)ₙNHCHMCO, (CH₂)ₙNHCOCHMNH, (CH₂)OCOCHMNH, (CH₂CH₂O)ₙCH₂CH₂NHCHMCO, (CH₂CH₂O)ₙCH₂CH₂CONHCHMCO, (CH₂CH₂O)ₙCH₂CH₂NHCHMCO, (CH₂CH₂O)ₙCH₂CH₂NHCOCHMNH, (CH₂CH₂O)ₙCOCHMNH, where n is 0-25 and where M is a side chain selected from the group consisting of side chains of the natural amino acids: H, CH₃, CH₂SH, CH₂COOH, CH₂CH₂COOH, CH₂C₆H₅, CH₂C₃H₃N₂, CH(CH₃)CH₂CH₃, (CH₂)₄NH₂, CH₂CH(CH₃)₂, CH₂CH₂SCH₃, CH₂CONH₂, (CH₂)₄NHCOC₄H₅NCH₃, CH₂CH₂CH₂, CH₂CH₂CONH₂, (CH₂)₃NH—C(NH)NH₂, CH₂OH, CH(OH)CH₃, CH₂SeH, CH(CH₃)₂, CH₂C₈H₆N, CH₂C₆H₄OH;
and where the targeting vector is an antibody, a nanobody, a polymer, a nanomedicine, a cell, a protein, a peptide, or a small molecule.

Examples of unsymmetrical Tzs that are suitable for ligation in step b) of the method for providing a labeled single isomeric chemical entity targeting vector:

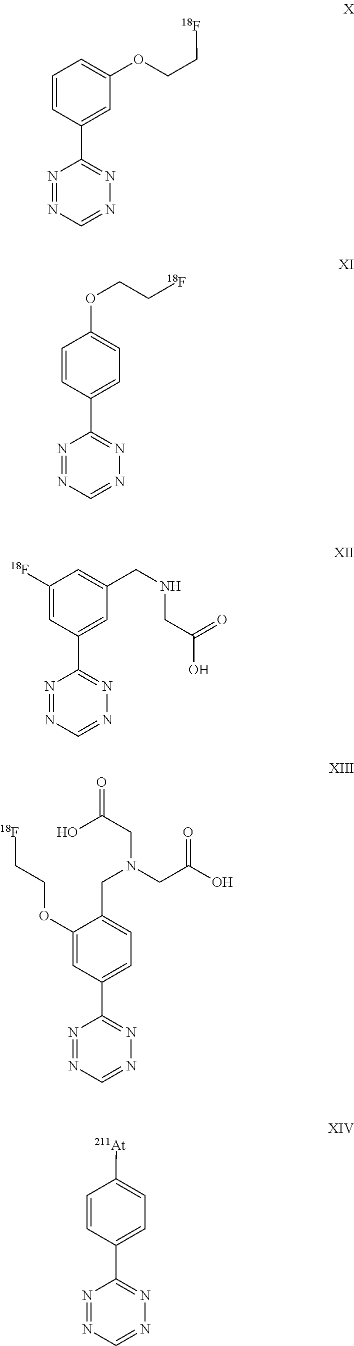

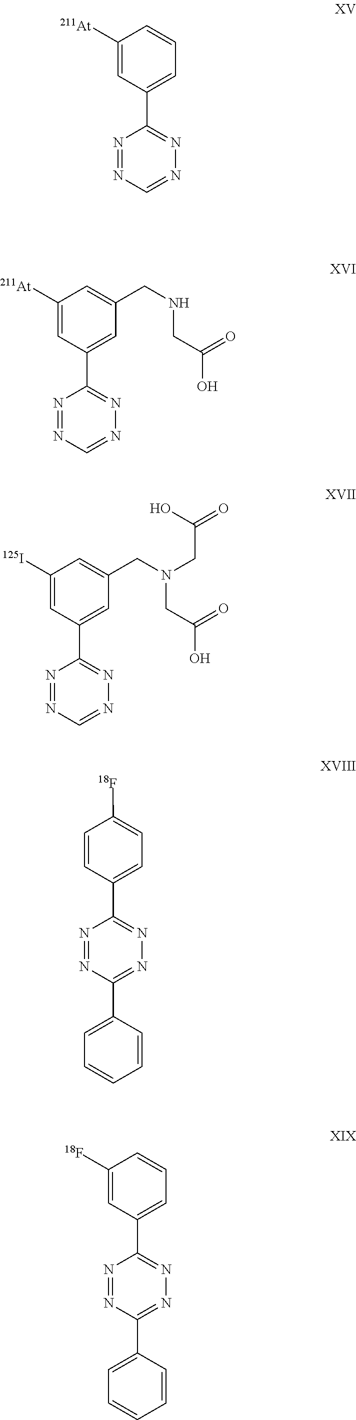

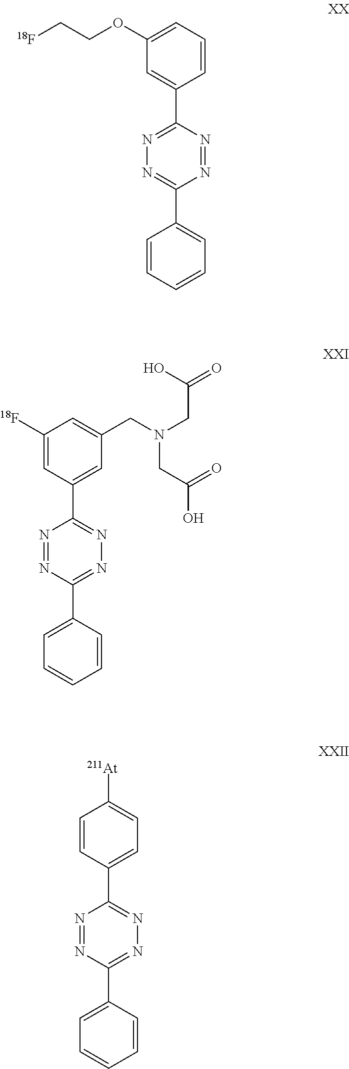

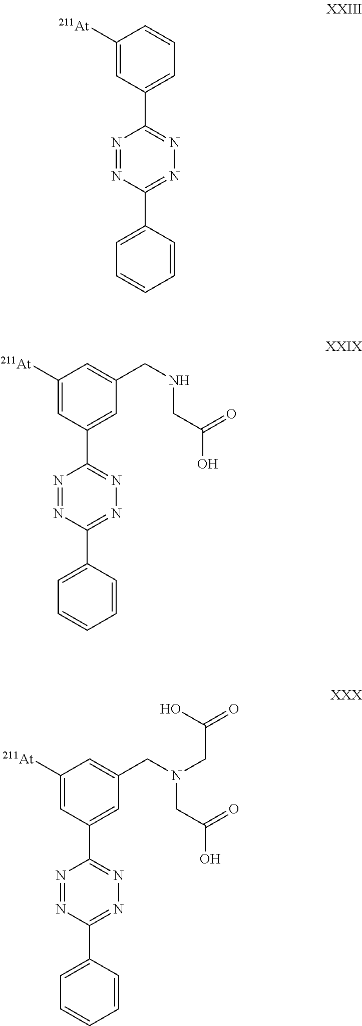

Examples of monocyclic isomer-free TCH and TCN that are suitable for ligation in step b) of the method for providing a labeled single isomeric chemical entity targeting vector:

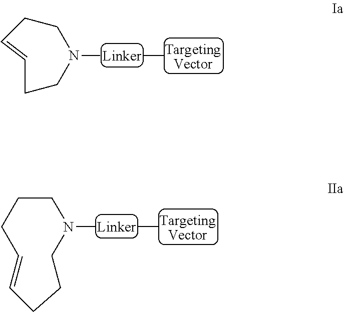

Wherein the linker is selected from the group comprising: —(CH₂)ₙ— (CH₂)ₙNH, (CH₂)ₙCO, (CH₂)ₙO, (CH₂CH₂O)ₙ, (CH₂CH₂O)ₙCH₂CH₂NH, (CH₂CH₂O)ₙCH₂CH₂CO, —CO(CH)₂— CO(CH₂)ₙNH, CO(CH₂)ₙCO, CO(CH₂)ₙO, CO(CH₂CH₂O)ₙ CO(CH₂CH₂O)ₙCH₂CH₂NH, CO(CH₂CH₂O)ₙCH₂CH₂CO, COO(CH)₂—COO(CH₂)ₙNH, COO(CH₂)ₙCO, COO(CH₂)ₙO, COO(CH₂CH₂O)ₙ COO(CH₂CH₂O)ₙCH₂CH₂NH, COO(CH₂CH₂O)ₙCH₂CH₂CO, CONH(CH)₂—CONH(CH₂)ₙNH, CONH(CH₂)ₙCO, CONH(CH₂)ₙO, CONH(CH₂CH₂O)ₙ, CONH(CH₂CH₂O)ₙCH₂CH₂NH, CONH(CH₂CH₂O)ₙCH₂CH₂CO, —CONHPhCO, —COOPhCO, —COPhCO, CONHCHMCO, (CH₂)ₙNHCHMCO, (CH₂)ₙOCONHCHMCO, (CH₂)ₙNHCHMCO, (CH₂)ₙNHCOCHMNH, (CH₂)OCOCHMNH, (CH₂CH₂O)ₙCH₂CH₂NHCHMCO, (CH₂CH₂O)ₙCH₂CH₂CONHCHMCO, (CH₂CH₂O)ₙCH₂CH₂NHCHMCO, (CH₂CH₂O)ₙCH₂CH₂NHCOCHMNH, (CH₂CH₂O)ₙCOCHMNH, where n is 0-25 and where M is a side chain selected from the group consisting of side chains of the natural amino acids: H, CH₃, CH₂SH, CH₂COOH, CH₂CH₂COOH, CH₂C₆H₅, CH₂C₃H₃N₂, CH(CH₃)CH₂CH₃, (CH₂)₄NH₂, CH₂CH(CH₃)₂, CH₂CH₂SCH₃, CH₂CONH₂, (CH₂)₄NHCOC₄H₅NCH₃, CH₂CH₂CH₂, CH₂CH₂CONH₂, (CH₂)₃NH—C(NH)NH₂, CH₂OH, CH(OH)CH₃, CH₂SeH, CH(CH₃)₂, CH₂C₈H₆N, CH₂C₆H₄OH; and where the targeting vector is an antibody, a nanobody, a polymer, a nanomedicine, a cell, a protein, a peptide, or a small molecule.

Examples of bicyclic isomer-free TCOs that are suitable for ligation in step b) of the method for providing a labeled single isomeric chemical entity targeting vector:

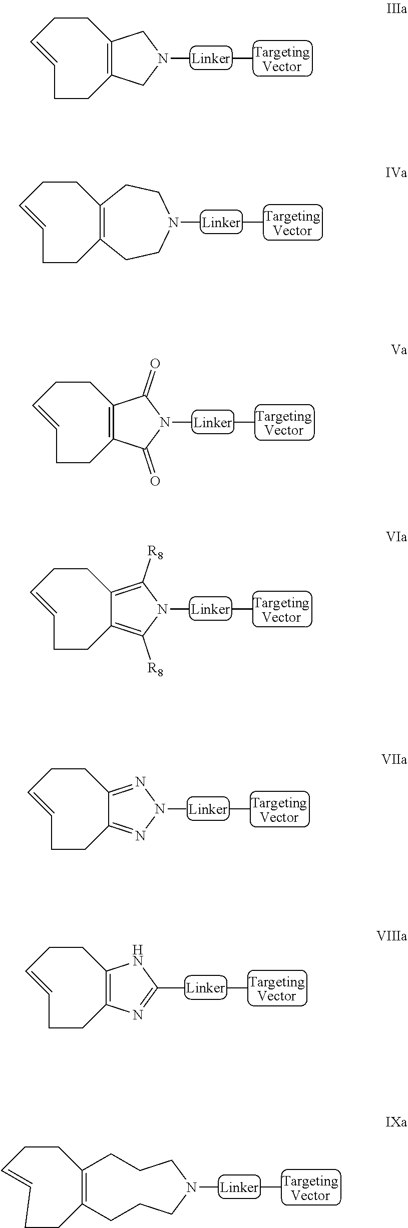

Wherein R₃ is H, F, OH, NH₂, CH₃, COOH, COOCH₃, CF₃, Cl, CONH₂, CONHCH₃, CON(CH₃)₂, CH₂CH₂OH, CH₂CH₂NH₂, CHCH₂N(CH₃)₂, and the linker is selected from the group comprising: —(CH₂)ₙ—(CH₂)ₙNH, (CH₂)ₙCO, (CH₂)ₙO, (CH₂CH₂O)ₙ, (CH₂CH₂O)ₙCH₂CH₂NH, (CH₂CH₂O)ₙCH₂CH₂CO, —CO(CH)₂—CO(CH₂)ₙNH, CO(CH₂)ₙCO, CO(CH₂)ₙO, CO(CH₂CH₂O)ₙ CO(CH₂CH₂O)ₙCH₂CH₂NH, CO(CH₂CH₂O)ₙCH₂CH₂CO, COO(CH)₂—COO(CH₂)ₙNH, COO(CH₂)ₙCO, COO(CH₂)ₙO, COO(CH₂CH₂O)ₙ COO(CH₂CH₂O)ₙCH₂CH₂NH, COO(CH₂CH₂O)ₙCH₂CH₂CO, CONH(CH)₂—CONH(CH₂)ₙNH, CONH(CH₂)ₙCO, CONH(CH₂)ₙO, CONH(CH₂CH₂O)ₙ, CONH(CH₂CH₂O)ₙCH₂CH₂NH, CONH(CH₂CH₂O)ₙCH₂CH₂CO, —CONHPhCO, —COOPhCO, —COPhCO, CONHCHMCO, (CH₂)ₙNHCHMCO, (CH₂)ₙOOONHCHMCO, (CH₂)ₙNHCHMCO, (CH₂)ₙNHCOCHMNH, (CH₂)OCOCHMNH, (CH₂CH₂O)ₙCH₂CH₂NHCHMCO, (CH₂CH₂O)ₙCH₂CH₂CONHCHMCO, (CH₂CH₂O)ₙCH₂CH₂NHCHMCO, (CH₂CH₂O)ₙCH₂CH₂NHCOCHMNH, (CH₂CH₂O)ₙCOCHMNH, where n is 0-25 and where M is a side chain selected from the group consisting of side chains of the natural amino acids: H, CH₃, CH₂SH, CH₂COOH, CH₂CH₂COOH, CH₂C₆H₅, CH₂C₃H₃N₂, CH(CH₃)CH₂CH₃, (CH₂)₄NH₂, CH₂CH(CH₃)₂, CH₂CH₂SCH₃, CH₂CONH₂, (CH₂)₄NHCOC₄H₅NCH₃, CH₂CH₂CH₂, CH₂CH₂CONH₂, (CH₂)₃NH—C(NH)NH₂, CH₂OH, CH(OH)CH₃, CH₂SeH, CH(CH₃)₂, CH₂C₈H₆N, CH₂C₆H₄OH; and where the targeting vector is an antibody, a nanobody, a polymer, a nanomedicine, a cell, a protein, a peptide, or a small molecule.

Examples of tricyclic isomer-free TCOs that are suitable for ligation in step b) of the method for providing a labeled single isomeric chemical entity targeting vector:

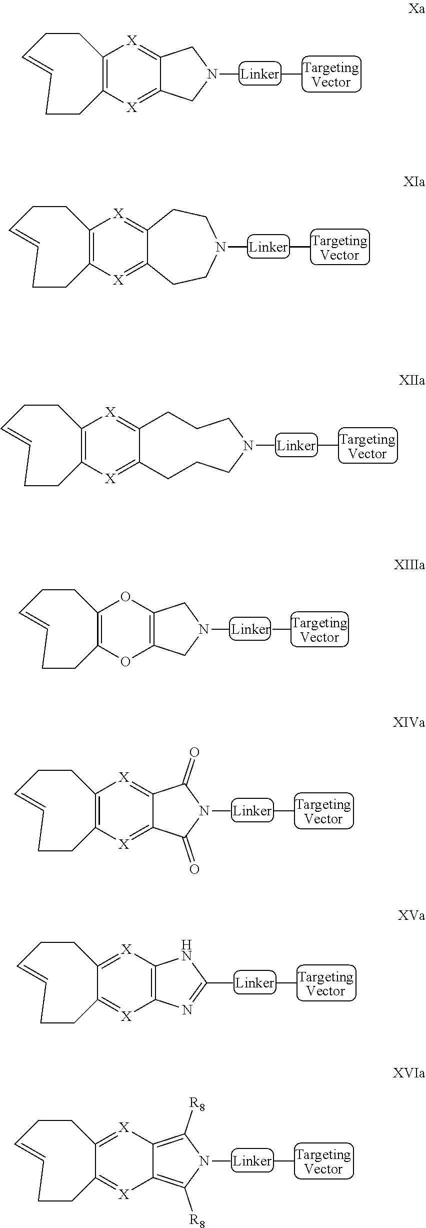

wherein X is N, NO or CR₃; Y is N, NO or CR₃; R₃ is selected from the group consisting of: —H, —F, —OH, —NH₂, —COOH, —COOCH₃, CF₃, —Cl, —CONH₂, CONHCH₃, —CON(CH₃)₂, —CH₂CH₂OH, —CH₂CH₂NH₂, —CHCH₂N(CH₃)₂ and
wherein the linker is selected from the group comprising: —(CH₂)ₙ—(CH₂)ₙNH, (CH₂)ₙCO, (CH₂)ₙO, (CH₂CH₂O)ₙ, (CH₂CH₂O)ₙCH₂CH₂NH, (CH₂CH₂O)ₙCH₂CH₂CO, —CO(CH)₂—CO(CH₂)ₙNH, CO(CH₂)ₙCO, CO(CH₂)ₙO, CO(CH₂CH₂O)ₙ CO(CH₂CH₂O)ₙCH₂CH₂NH, CO(CH₂CH₂O)ₙCH₂CH₂CO, COO(CH)₂—COO(CH₂)ₙNH, COO(CH₂)ₙCO, COO(CH₂)ₙO, COO(CH₂CH₂O)ₙ COO(CH₂CH₂O)ₙCH₂CH₂NH, COO(CH₂CH₂O)ₙCH₂CH₂CO, CONH(CH)₂—CONH(CH₂)ₙNH, CONH(CH₂)ₙCO, CONH(CH₂)ₙO, CONH(CH₂CH₂O)ₙ, CONH(CH₂CH₂O)ₙCH₂CH₂NH, CONH(CH₂CH₂O)ₙCH₂CH₂CO, —CONHPhCO, —COOPhCO, —COPhCO, CONHCHMCO, (CH₂)ₙNHCHMCO, (CH₂)ₙOCONHCHMCO, (CH₂)ₙNHCHMCO, (CH₂)ₙNHCOCHMNH, (CH₂)OCOCHMNH, (CH₂CH₂O)ₙCH₂CH₂NHCHMCO, (CH₂CH₂O)ₙCH₂CH₂CONHCHMCO, (CH₂CH₂O)ₙCH₂CH₂NHCHMCO, (CH₂CH₂O)ₙCH₂CH₂NHCOCHMNH, (CH₂CH₂O)ₙCOCHMNH, where n is 0-25 and where M is a side chain selected from the group consisting of side chains of the natural amino acids: H, CH₃, CH₂SH, CH₂COOH, CH₂CH₂COOH, CH₂C₆H₅, CH₂C₃H₃N₂, CH(CH₃)CH₂CH₃, (CH₂)₄NH₂, CH₂CH(CH₃)₂, CH₂CH₂SCH₃, CH₂CONH₂, (CH₂)₄NHCOC₄H₅NCH₃, CH₂CH₂CH₂, CH₂CH₂CONH₂, (CH₂)₃NH—C(NH)NH₂, CH₂OH, CH(OH)CH₃, CH₂SeH, CH(CH₃)₂, CH₂C₈H₆N, CH₂C₆H₄OH;
and where the targeting vector is an antibody, a nanobody, a polymer, a nanomedicine, a cell, a protein, a peptide, or a small molecule.

Examples of tetracyclic isomer-free TCO that are suitable for ligation in step b) of the method for providing a labeled single isomeric chemical entity targeting vector:

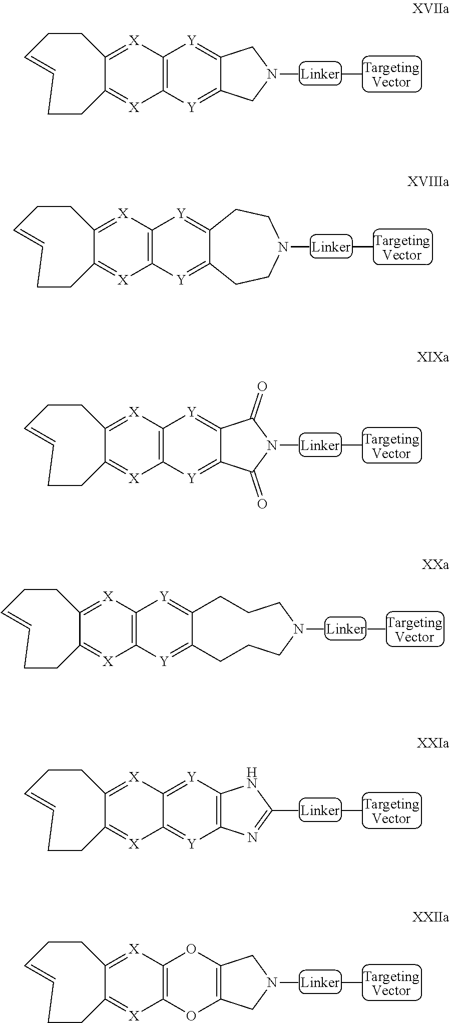

wherein X is N, NO or CR₃; Y is N, NO or CR₃; R₃ is selected from the group consisting of: —H, —F, —OH, —NH₂, —COOH, —COOCH₃, CF₃, —Cl, —CONH₂, CONHCH₃, —CON(CH₃)₂, —CH₂CH₂OH, —CH₂CH₂NH₂, —CHCH₂N(CH₃)₂ and the linker is selected from the group comprising: —(CH₂)ₙ—(CH₂)ₙNH, (CH₂)ₙCO, (CH₂)ₙO, (CH₂CH₂O)ₙ, (CH₂CH₂O)ₙCH₂CH₂NH, (CH₂CH₂O)ₙCH₂CH₂CO, —CO(CH)₂—CO(CH₂)ₙNH, CO(CH₂)ₙCO, CO(CH₂)ₙO, CO(CH₂CH₂O)ₙ CO(CH₂CH₂O)ₙCH₂CH₂NH, CO(CH₂CH₂O)ₙCH₂CH₂CO, COO(CH)₂—COO(CH₂)ₙNH, COO(CH₂)ₙCO, COO(CH₂)ₙO, COO(CH₂CH₂O)ₙ COO(CH₂CH₂O)ₙCH₂CH₂NH, COO(CH₂CH₂O)ₙCH₂CH₂CO, CONH(CH)₂—CONH(CH₂)ₙNH, CONH(CH₂)ₙCO, CONH(CH₂)ₙO, CONH(CH₂CH₂O)ₙ, CONH(CH₂CH₂O)ₙCH₂CH₂NH, CONH(CH₂CH₂O)ₙCH₂CH₂CO, —CONHPhCO, —COOPhCO, —COPhCO, CONHCHMCO, (CH₂)ₙNHCHMCO, (CH₂)ₙOOONHCHMCO, (CH₂)ₙNHCHMCO, (CH₂)ₙNHCOCHMNH, (CH₂)OCOCHMNH, (CH₂CH₂O)ₙCH₂CH₂NHCHMCO, (CH₂CH₂O)ₙCH₂CH₂CONHCHMCO, (CH₂CH₂O)ₙCH₂CH₂NHCHMCO, (CH₂CH₂O)ₙCH₂CH₂NHCOCHMNH, (CH₂CH₂O)ₙCOCHMNH, where n is 0-25 and where M is a side chain selected from the group consisting of side chains of the natural amino acids: H, CH₃, CH₂SH, CH₂COOH, CH₂CH₂COOH, CH₂C₆H₅, CH₂C₃H₃N₂, CH(CH₃)CH₂CH₃, (CH₂)₄NH₂, CH₂CH(CH₃)₂, CH₂CH₂SCH₃, CH₂CONH₂, (CH₂)₄NHCOC₄H₅NCH₃, CH₂CH₂CH₂, CH₂CH₂CONH₂, (CH₂)₃NH—C(NH)NH₂, CH₂OH, CH(OH)CH₃, CH₂SeH, CH(CH₃)₂, CH₂C₈H₆N, CH₂C₆H₄OH;
and where the targeting vector is an antibody, a nanobody, a polymer, a nanomedicine, a cell, a protein, a peptide, or a small molecule.

Step c) in the method for providing a labeled single isomeric chemical entity targeting vector is an oxidation step. Even though auto-oxidation of the ligated entity targeting vector, such as a pyridazine, obtained in step b) of the method occurs spontaneously, this process is extremely slow and can last from several hours up to several days. Step c) in the method provides a fast way for oxidizing the pyridazine compound wherein only a single isomer form is obtained at least within 60 minutes, such as within 1-20 minutes. In order to facilitate this process, the dihydropyridazines are oxidized by either a standard, or solid-supported oxidant, preferably solid-supported. The oxidizing step can be performed at a temperature ranging from 15 to 50° C., such as at 20-30° C., preferably at room temperature, for approximately 10 to 60 minutes, preferably for less than 20 minutes. To facilitate the oxidation adding 1 to 100 equivalents, preferably 1, of an oxidant to the ligated compound obtained from the ligation step. The oxidant needs to be selective for the oxidation of the dihydropyrazine to pyridazine (95% efficiency). The targeting vector must not be chemically modified by the oxidant. The oxidant is a quinone oxidant selected from the group comprising: chloranil, fluoranil, DDQ, NaNO₂.

In a preferred embodiment, the diene is a symmetrical substituted diene wherein at least one of the symmetry planes passes through the nitrogen-nitrogen bonds of at least one tetrazine ring obtained from a precursor selected from:

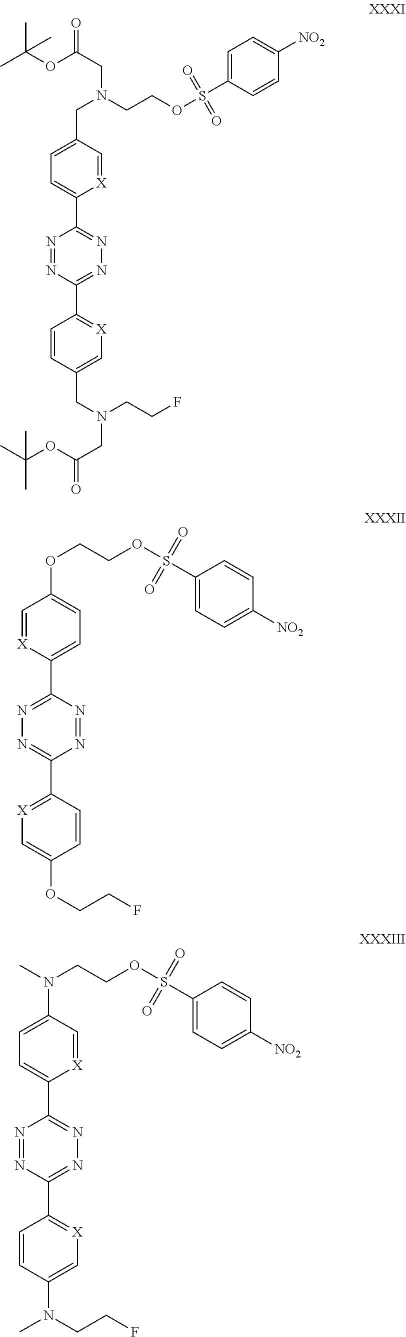

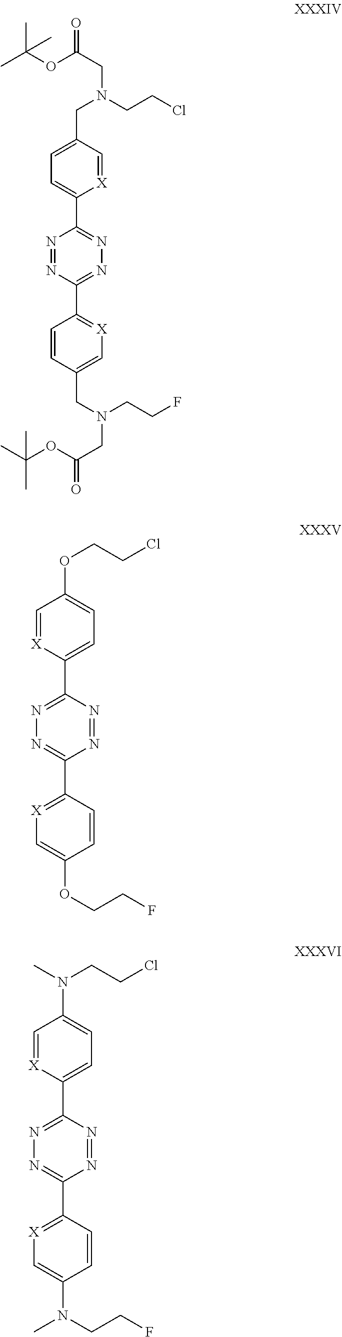

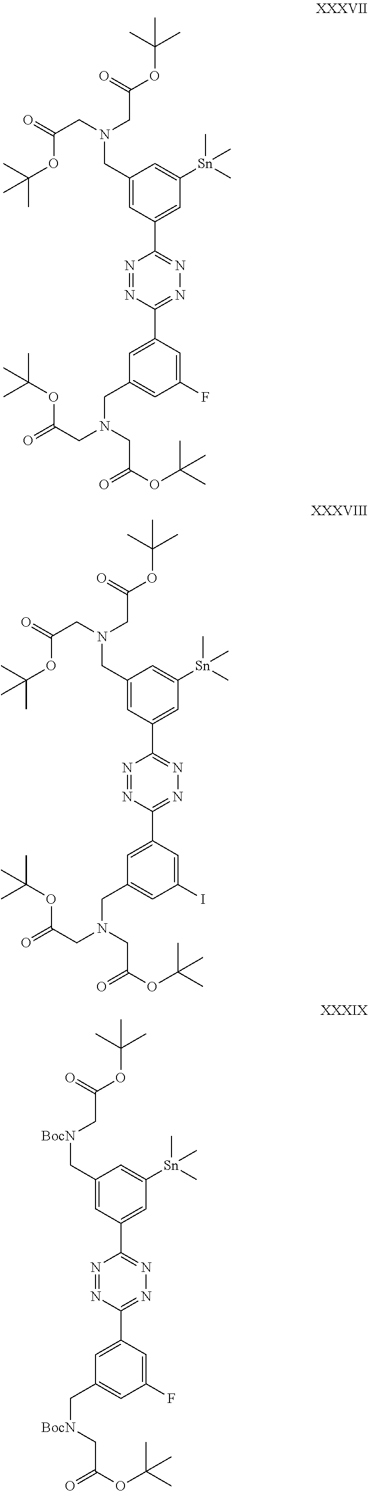

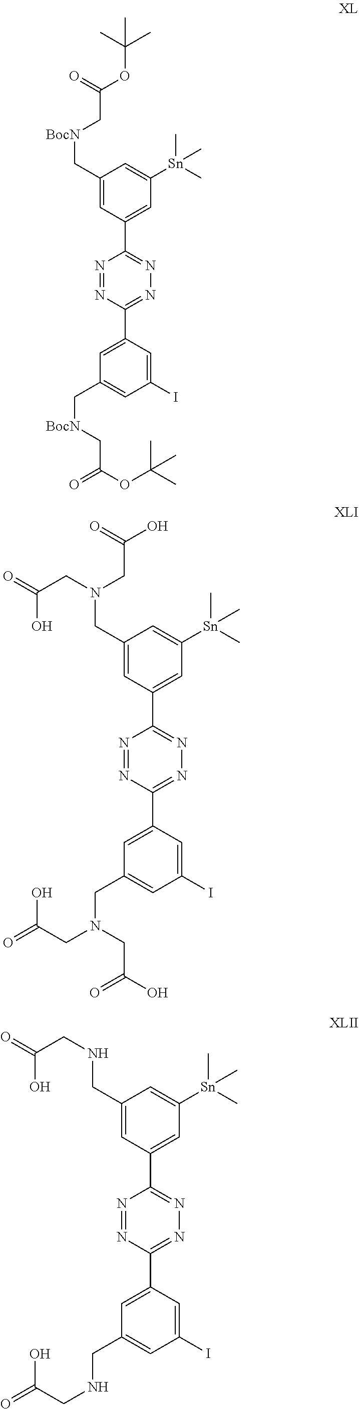

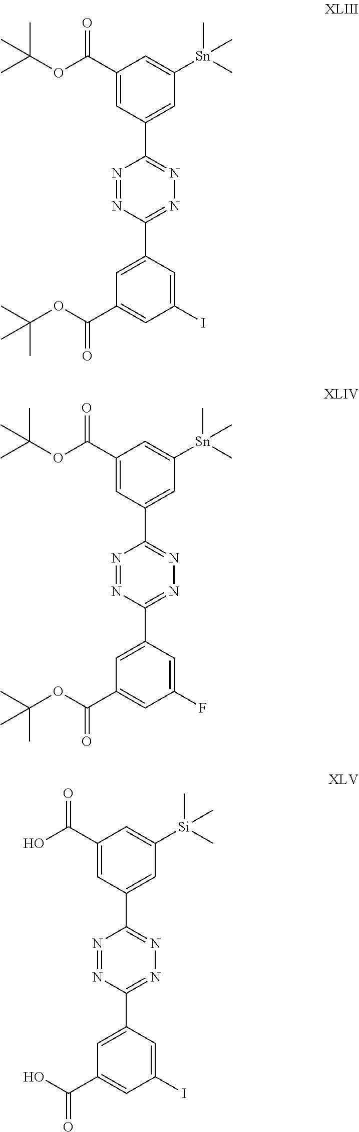

wherein X is CH or N.

In another preferred embodiment, the diene is an unsymmetrical substituted diene obtained from a precursor selected from:

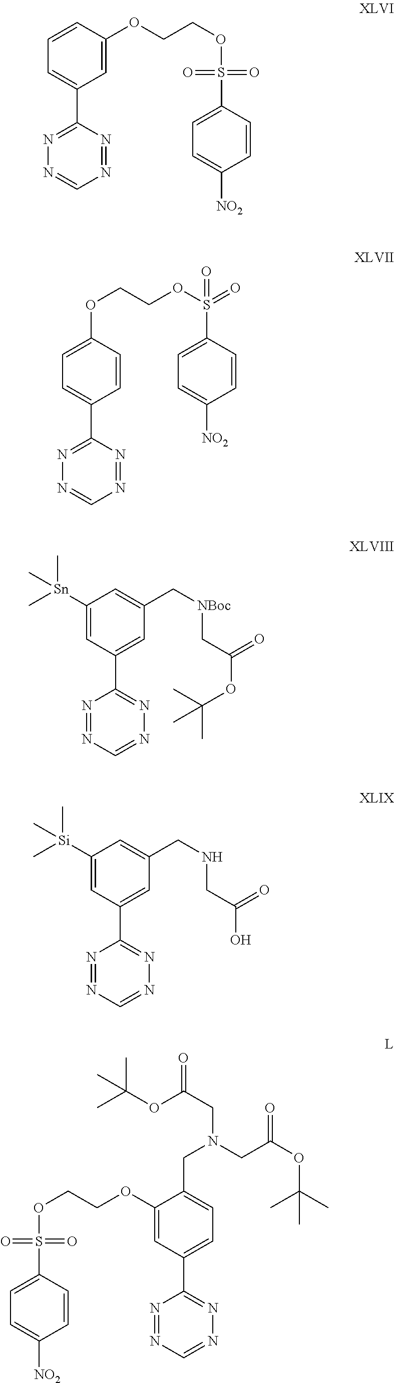 [SMILES: O=[N+]([O-])c1ccc(S(=O)(=O)OCCOc2cccc(-c3nncnn3)c2)cc1 ; O=[N+]([O-])c1ccc(S(=O)(=O)OCCOc2ccc(-c3nncnn3)cc2)cc1 ; CC(C)(C)OC(=O)CN(Cc1cc(-c2nncnn2)c[c]([Sn]([CH3])([CH3])[CH3])c1)C(=O)OC(C)(C)C ; C[Si](C)(C)c1cc(CNCC(=O)O)cc(-c2nncnn2)c1 ; CC(C)(C)OC(=O)CN(CC(=O)OC(C)(C)C)Cc1ccc(-c2nncnn2)cc1OCCOS(=O)(=O)c1ccc([N+](=O)[O-])cc1]

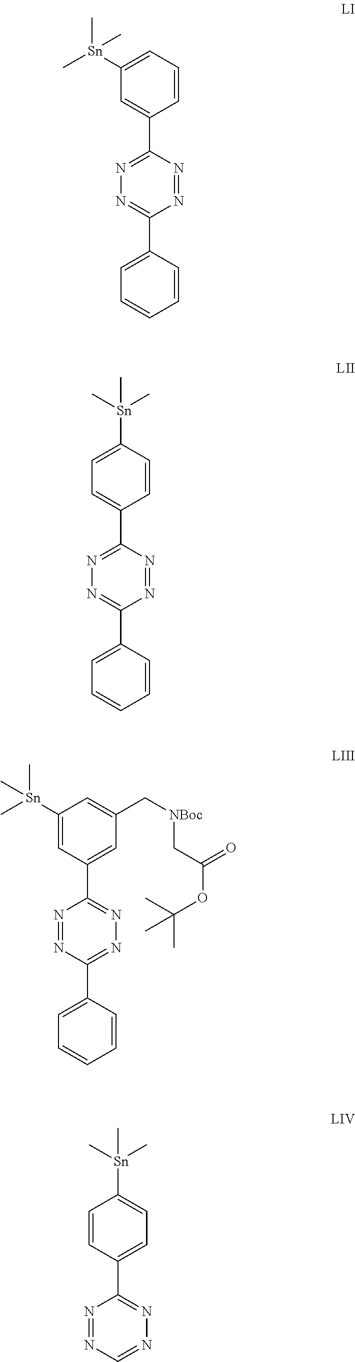 [SMILES: [CH3][Sn]([CH3])([CH3])[c]1cccc(-c2nnc(-c3ccccc3)nn2)c1 ; [CH3][Sn]([CH3])([CH3])[c]1ccc(-c2nnc(-c3ccccc3)nn2)cc1 ; CC(C)(C)OC(=O)CN(Cc1cc(-c2nnc(-c3ccccc3)nn2)c[c]([Sn]([CH3])([CH3])[CH3])c1)C(=O)OC(C)(C)C ; [CH3][Sn]([CH3])([CH3])[c]1ccc(-c2nncnn2)cc1]

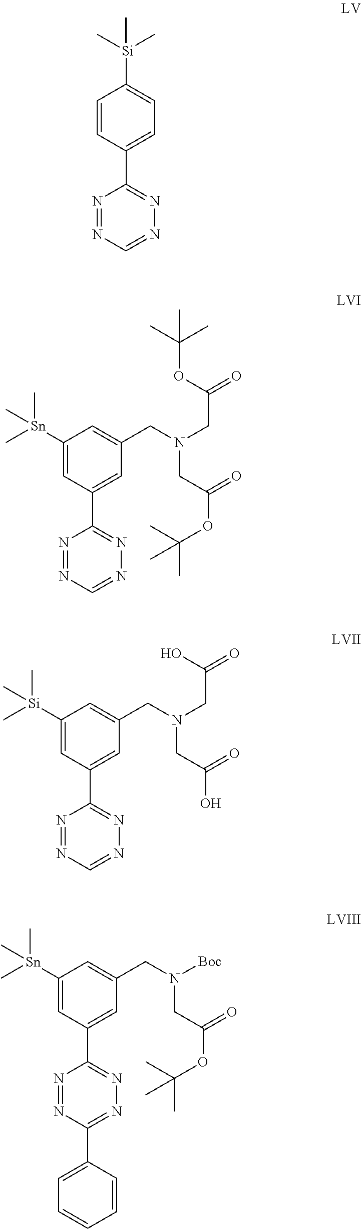 [SMILES: C[Si](C)(C)c1ccc(-c2nncnn2)cc1 ; CC(C)(C)OC(=O)CN(CC(=O)OC(C)(C)C)Cc1cc(-c2nncnn2)c[c]([Sn]([CH3])([CH3])[CH3])c1 ; C[Si](C)(C)c1cc(CN(CC(=O)O)CC(=O)O)cc(-c2nncnn2)c1 ; CC(C)(C)OC(=O)CN(Cc1cc(-c2nnc(-c3ccccc3)nn2)c[c]([Sn]([CH3])([CH3])[CH3])c1)C(=O)OC(C)(C)C]

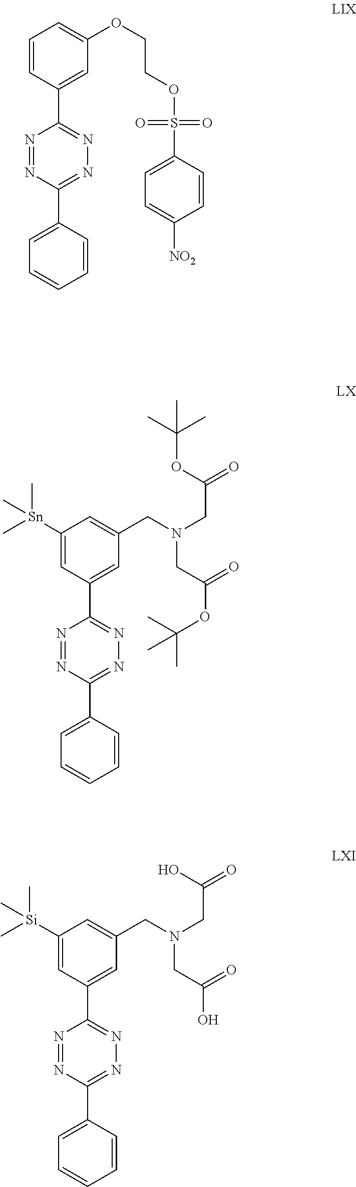 [SMILES: O=[N+]([O-])c1ccc(S(=O)(=O)OCCOc2cccc(-c3nnc(-c4ccccc4)nn3)c2)cc1 ; CC(C)(C)OC(=O)CN(CC(=O)OC(C)(C)C)Cc1cc(-c2nnc(-c3ccccc3)nn2)c[c]([Sn]([CH3])([CH3])[CH3])c1 ; C[Si](C)(C)c1cc(CN(CC(=O)O)CC(=O)O)cc(-c2nnc(-c3ccccc3)nn2)c1]

In another preferred embodiment, the isomer-free dienophile is obtained from a precursor selected from:

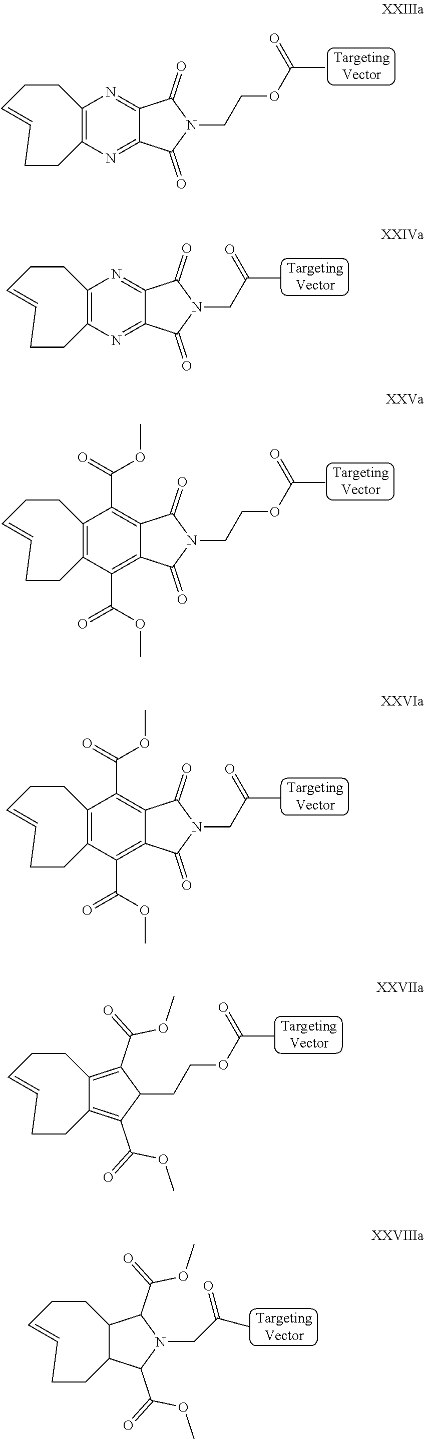 [SMILES: CC(=O)OCCN1C(=O)c2nc3c(nc2C1=O)CC/C=C/CC3 ; CC(=O)CN1C(=O)c2nc3c(nc2C1=O)CC/C=C/CC3 ; COC(=O)c1c2c(c(C(=O)OC)c3c1C(=O)N(CCOC(C)=O)C3=O)CC/C=C/CC2 ; COC(=O)c1c2c(c(C(=O)OC)c3c1C(=O)N(CC(C)=O)C3=O)CC/C=C/CC2 ; COC(=O)C1=C2CC/C=C/CCC2=C(C(=O)OC)C1CCOC(C)=O ; COC(=O)C1C2CC/C=C/CCC2C(C(=O)OC)N1CC(C)=O]

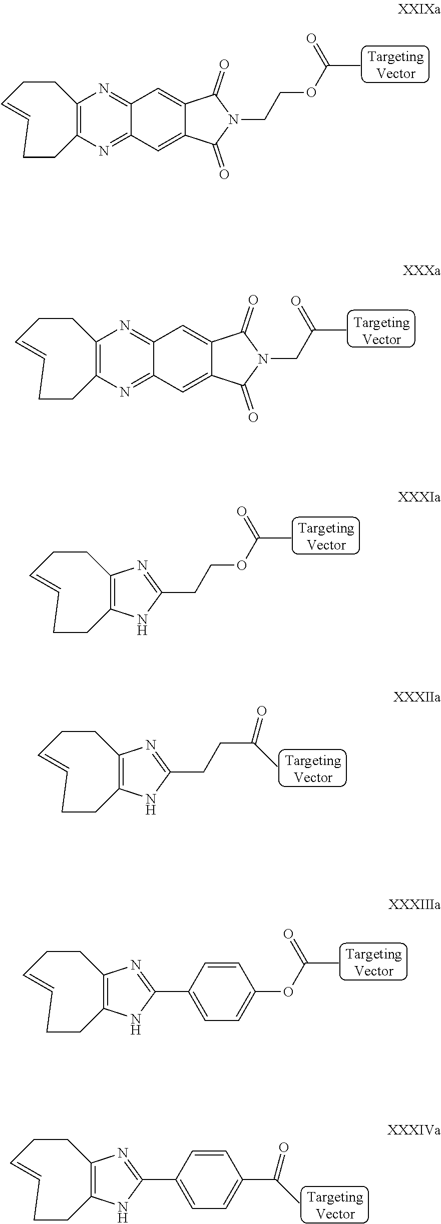 [SMILES: CC(=O)OCCN1C(=O)c2cc3nc4c(nc3cc2C1=O)CC/C=C/CC4 ; CC(=O)CN1C(=O)c2cc3nc4c(nc3cc2C1=O)CC/C=C/CC4 ; CC(=O)OCCc1nc2c([nH]1)CC/C=C/CC2 ; CC(=O)CCc1nc2c([nH]1)CC/C=C/CC2 ; CC(=O)Oc1ccc(-c2nc3c([nH]2)CC/C=C/CC3)cc1 ; CC(=O)c1ccc(-c2nc3c([nH]2)CC/C=C/CC3)cc1]

wherein the targeting vector is an antibody, a nanobody, a polymer, a nanomedicine, a cell, a protein, a peptide, or a small molecule.

The labeled single isomeric chemical entity targeting vector provided by the method for providing a labeled single isomeric chemical entity targeting vectors can be used in therapy, radiotherapy, theranostics, diagnostics, or imaging, depending on the labeling agent, or the pharmaceutical agent, or imaging agent or therapeutic agent and on the targeting vector.

Preferably, the targeting vector is coupled to the linker via a nitrogen on the targeting vector. Alternatively, the targeting vector is preferable coupled to the linker via a carbonyl on the targeting vector. In a preferred embodiment, the labeled single isomeric chemical entity targeting vector provided by the method for providing a labeled single isomeric chemical entity targeting vectors is used in therapy.

In another preferred embodiment, the labeled single isomeric chemical entity targeting vector provided by the method for providing a labeled single isomeric chemical entity targeting vectors is used in radiotherapy.

In another preferred embodiment, the labeled single isomeric chemical entity targeting vector provided by the method for providing a labeled single isomeric chemical entity targeting vectors is used in theranostics.

In another preferred embodiment, the labeled single isomeric chemical entity targeting vector provided by the method for providing a labeled single isomeric chemical entity targeting vectors is used in diagnostics.

In another preferred embodiment, the labeled single isomeric chemical entity targeting vector provided by the method for providing a labeled single isomeric chemical entity targeting vectors is used in imaging.

The following Examples describes (1) the synthesis of tetrazines and TCOs representative for use in step a) and b) of the present method for providing a labeled single isomeric chemical entity targeting vector and (2) click reactions and oxidations between such compounds, yielding a single isomeric pyridazine.

### EXAMPLES

### General

All reagents and solvents were dried prior to use according to standard methods. Commercial reagents were used without further purification. Analytical TLC was performed using silica gel 60 F254 (Merck) with detection by UV absorption and/or by charring following immersion in a 7% ethanolic solution of sulfuric acid or KMnO₄-solution (1.5 g of KMnO₄, 10 g K₂CO₃, and 1.25 mL 10% NaOH in 200 mL water). Purification of compounds was carried out by column chromatography on silica gel (40-60 μm, 60 Å) or employing a CombiFlash NextGen 300+(Teledyne ISCO). ¹H and ¹³C NMR spectra were recorded on Brucker (400 and 600 MHz instruments), using Chloroform-d, Methanol-d₄ or DMSO-d₆ as deuterated solvent and with the residual solvent as the internal reference. For all NMR experiences the deuterated solvent signal was used as the internal lock. Chemical shifts are reported in δ parts per million (ppm). Coupling constants (J values) are given in Hertz (Hz). Multiplicities of ¹H NMR signals are reported as follows: s, singlet; d, doublet; dd, doublet of doublets; ddd, doublet of doublets of doublets; dt, doublet of triplets; t, triplet; q, quartet; m, multiplet; br, broad signal. NMR spectra of all compounds are reprocessed in MestReNova software (version 12.0.22023) from original FID's files. Mass spectra analysis was performed using MS-Acquity-A: Waters Acquity UPLC with QDa-detector. Purification by preparative HPLC was performed on Agilent 1260 infinity system, column SymmetryPrep-C18, 17 mL/min H₂O-MeCN gradient 50-100% 15 min with 0.1% trifluoroacetic acid. All final compounds were >95% pure as determined by analytical HPLC. Analytical HPLC method: (Thermo Fisher® UltiMate 3000) with a C-18 column (Luna@5u C18(2) 100 Å, 150×4.6 mm), eluents: A: H2O with 0.1% TFA, B: MeCN with 0.1% TFA. Gradient from 100% A->100% B over 15 minutes, back to 100% A over 4 minutes, flow rate 1.5 mL/min. Detection by UV-absorption at A=254 nm on a UVD 170U detector.

### Example 1

Synthesis of Symmetrical Tetrazines and their Precursors

### Compound I and XXXIV

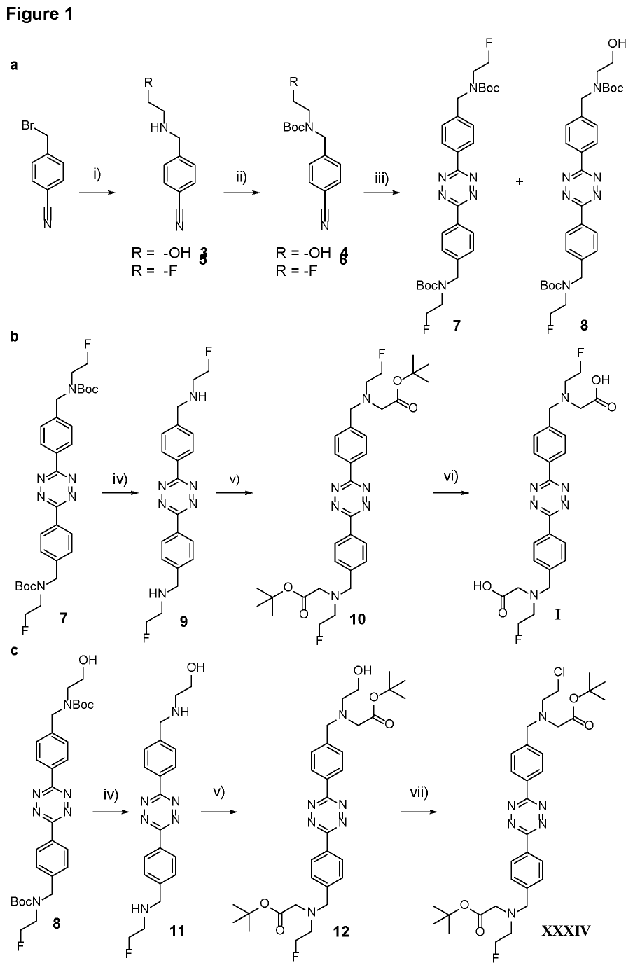

FIG. 1. shows a reaction scheme for the synthesis of symmetrical tetrazines. Reagents and conditions: i) NH₂(CH₂)₂R, MeCN, 12 h, rt; ii) Boc₂O, Et₃N, DCM, 12 h, rt; iii) Zn(OTf)₂, NH₂NH₂, H₂O, EtOH, 65° C., 24 h; iv) HCl, dioxane, rt, 4 h; v) t-Butyl bromoacetate, Et₃N, DMF, 50° C., 12 h; vi) TFA, DCM, rt, 2 h; vii) MsCI, Et₃N, DMAP, DCM, rt, 12 h.

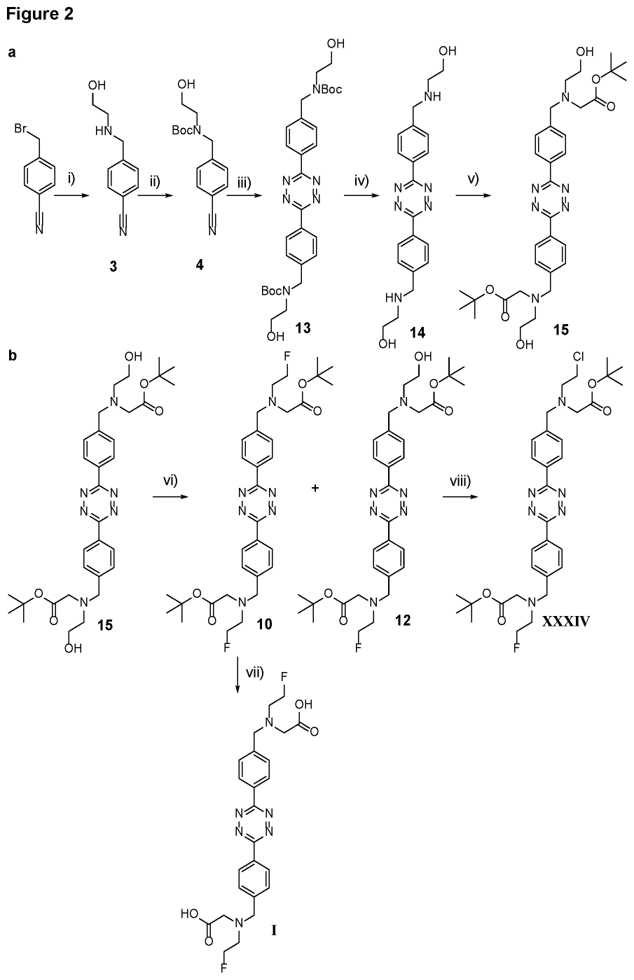

FIG. 2. Reagents and conditions: i) NH₂(CH₂)₂₀H, MeCN, 12 h, rt; ii) Boc₂O, Et₃N, DCM, 12 h, rt; iii) Zn(OTf)₂, NH₂NH₂—H₂O, EtOH, 65° C., 24 h; iv) HCl, dioxane, rt, 4 h; v) t-Butyl bromoacetate, Et₃N, DMF, 50° C., 12 h; vi) DAST, DCM, −78° C. to rt, 4 h; vii) TFA, DCM, rt, 2 h; viii) MsCI, Et₃N, DMAP, DCM, rt, 12 h.

### Synthesis of 4-(((2-hydroxyethyl)amino)methyl)benzonitrile (3)

To a solution of ethanolamine in DCM (60 mL) was added dropwise a solution of 4-(bromomethyl)benzonitrile (4 gr, 20.20 mmol) in DCM (20 mL). The reaction was stirred at rt for 1 h. The organic phase was then washed with water (3×30 mL), dried and concentrated under reduced pressure to give 3.55 g (99%) of the desired product as a white solid. Rf=0.21 (DCM/MeOH 95/5); ¹H NMR (400 MHz, CDCl₃) δ 8.01-7.52 (m, 2H), 7.47-7.30 (m, 2), 4.05-3.76 (m, 2H), 3.74-3.43 (m, 2H), 2.73 (dtd, J=10.7, 6.0, 1.7 Hz, 2H), 2.47 (s, 1H); ¹³C NMR (101 MHz, CDCl₃) δ 145.76, 132.22, 128.67, 118.88, 110.71, 60.91, 53.07, 50.78.

### Synthesis of tert-butyl (4-cyanobenzyl)(2-hydroxyethyl)carbamate (4)

To a solution of 4-(((2-hydroxyethyl)amino)methyl)benzonitrile (1.62 g, 9.19 mmol) and Et₃N (2.56 mL, 18.39 mmol) in DCM (30 mL) was added Boc₂O (2.10 gr, 9.65 mmol). The reaction was stirred at room temperature for 12 h. The solution was then washed with water (50 mL) and K₂CO₃ saturated solution (50 mL), dried over anhydrous Na₂SO₄, filtered and concentrated under reduced pressure to afford 2.36 g (93%) of the desired product as a crude (mixture of rotamers). Rf.=0.4 (heptane/EtOAc 50/50); ¹H NMR (600 MHz, CDCl₃) δ 7.63 (d, J=7.9 Hz, 2H), 4.55-4.52 (m, 2H), 3.74 (s, 2H), 3.51-2.94 (m, 2H), 2.69 (s, 1H), 1.76-0.53 (m, 9H); ¹³C NMR (151 MHz, CDCl₃) δ 156.91, 144.11, 132.43, 128.02, 127.50, 118.72, 111.19, 81.02, 62.17, 61.46, 52.10, 51.17, 50.37, 49.51, 28.32.

### Synthesis of 4-(((2-fluoroethyl)amino)methyl)benzonitrile (5)

To a solution of 4-(bromomethyl)benzonitrile (0.78 g, 4.00 mmol) in CH₃CN (40 mL) was added K₂CO₃ (0.33 g, 24.0 mmol) and 2-fluoroethylamine hydrochloride (0.16 g, 16.0 mmol). The mixture was stirred at room temperature overnight. The solvent was removed under reduced pressure, and the residue was diluted with water (20 mL), extracted with EtOAc. The combined organic layer was washed with brine, dried over MgSO₄, filtered and concentrated under reduced pressure. The crude product was purified by flash column chromatography using EtOAc (Heptane/EtOAc 50/50) in heptane to afford 0.54 g (76%) of the desired product as a colorless oil. Rf=0.24 (Heptane/EtOAc 40/60). ¹H NMR (400 MHz, CDCl₃) δ 7.55 (d, J=8.2 Hz, 2H), 7.40 (d, J=8.0 Hz, 2H), 4.63-4.48 (m, 1H), 4.47-4.37 (m, 1H), 3.84 (s, 2H), 2.93-2.84 (m, 1H), 2.84-2.72 (m, 1H), 1.65 (s, 1H). ¹³C NMR (101 MHz, CDCl₃) δ 145.6, 132.3, 128.6, 118.9, 110.9, 83.5 (d, J=165.5 Hz), 53.1, 49.1 (d, J=19.7 Hz).

### Synthesis of tert-butyl 4-cyanobenzyl(2-fIuoroethyl)carbamate (6)

To a solution of 4-(((2-fluoroethyl)amino)methyl)benzonitrile (540 mg, 3.03 mmol) and Et₃N (1.27 mL, 9.09 mmol) in CH₂Cl₂ (40 mL) was added Boc₂O (790 mg, 3.63 mmol) and the mixture was stirred at room temperature for 12 h. The solution was washed with water and saturated K₂CO₃ solution, dried over Na₂SO₄, filtered and concentrated under reduced pressure. The crude product was purified by flash column chromatography using (Heptane/EtOAc 70/30) to afford 0.710 g (84%) of the desired product as a colorless oil (mixture of rotamers). Rf=0.42 (Heptane/EtOAc 80/20). ¹H NMR (400 MHz, CDCl₃) δ 7.55 (d, J=7.8 Hz, 2H), 7.27 (d, J=7.8 Hz, 2H), 4.79-4.10 (m, 4H), 3.62-3.28 (m, 2H), 1.96-1.05 (m, 9H). ¹³C NMR (101 MHz, CDCl₃) δ 155.4, 144.2, 143.8, 132.4, 128.1, 127.5, 118.7, 111.1, 83.2 (d, J=168.2 Hz), 82.7 (d, J=170.5 Hz), 52.1, 51.2, 47.7, 28.3.

### Synthesis of di-tert-butyl (((1,2,4,5-tetrazine-3,6-diyl)bis(4,1-phenylene))bis(methylene))bis((2-fluoroethyl)carbamate) (7) and tert-butyl (4-(6-(4-(((tert-butoxycarbonyl)(2-fluoroethyl)amino)methyl)phenyl)-1,2,4,5-tetrazin-3-yl)benzyl)(2-hydroxyethyl)carbamate (8)

To a suspension of tert-butyl 4-cyanobenzyl(2-fluoroethyl)carbamate (1.1 gr, 3.95 mmol), tert-butyl (4-cyanobenzyl)(2-hydroxyethyl)carbamate (0.27 gr, 0.99 mmol) and Zn(OTf)₂ (0.72 gr, 1.98 mmol) in EtOH (30 mL) was added hydrazine monohydrate (3.83 mL, 79 mmol). The mixture was allowed to stir at 70° C. for 22 hours, and when the reaction is completed, is cooled at room temperature. The volatiles were removed under reduced pressure and the residue solubilized in EtOH (40 mL). A solution of NaNO₂ (5.52 g, 80.00 mmol) in water (20 mL) was added to the crude reaction followed by dropwise addition of HCl (2M) until gas evolution ceased and a pH of 2-3 was achieved producing a red mixture. The crude reaction was extracted with DCM (3×40 mL) and washed with brine (3×20 mL). The organic phase was collected, dried over MgSO₄, filtered and concentrated under reduced pressure. Purification by flash chromatography (DCM/MeOH 98/2) afforded 0.300 g (26%) of di-tert-butyl (((1,2,4,5-tetrazine-3,6-diyl)bis(4,1-phenylene))bis(methylene))bis((2-fluoroethyl)carbamate) as a red oil (mixture of rotamers). Rf=0.45 (DCM/MeOH 98/2); ¹H NMR (600 MHz, CDC₃) δ 8.63 (d, J=8.0 Hz, 4H), 7.50 (d, J=7.0 Hz, 4H), 5.11-4.38 (m, 8H), 3.97-3.26 (m, 4H), 1.95-0.57 (m, 18H); ¹³C NMR (151 MHz, CDC₃) δ 163.77, 155.61, 155.57, 143.70, 130.81, 128.51, 128.21, 127.87, 83.18 (d, J=167.9 Hz), 82.61 (d, J=169.3 Hz), 72.44, 65.78, 52.09, 51.09, 47.67 (d, J=19.9 Hz), 47.19 (d, J=21.0 Hz), 28.38.

0.08 g (7%) of a second more polar fraction corresponding to tert-butyl (4-(6-(4-(((tert-butoxycarbonyl)(2-fluoroethyl)amino)methyl)phenyl)-1,2,4,5-tetrazin-3-yl)benzyl)(2-hydroxyethyl)carbamate was isolated as a red solid (mixture of rotamers). Rf=0.22 (DCM/MeOH 98/2); ¹H NMR (400 MHz, CDC₃) δ 8.54 (d, J=7.9 Hz, 4H), 7.40 (d, J=8.1 Hz, 4H), 4.79-4.34 (m, 8H), 3.52-3.25 (m, 5H), 1.38 (d, J=8.8 Hz, 18H).

### Synthesis of N,N′-(((1,2,4,5-tetrazine-3,6-diyl)bis(4,1-phenylene))bis(methylene))bis(2-fluoroethan-1-amine) (9)

Di-tert-butyl (((1,2,4,5-tetrazine-3,6-diyl)bis(4,1-phenylene))bis(methylene))bis((2-fluoroethyl) carbamate) (0.15 gr, 0.25 mmol) was treated with a solution of HCl (4 M) in dioxane (1 mL). A precipitate was formed. Filtration afforded 0.1 gr (83%) of the desired product as hydrochloride salt. ¹H NMR (600 MHz, DMSO) δ 9.70 (s, 4H), 8.61 (d, J=8.0 Hz, 4H), 7.89 (d, J=8.0 Hz, 4H), 4.86 (t, J=4.6 Hz, 2H), 4.78 (t, J=4.6 Hz, 2H), 4.37 (s, 4H), 3.40-3.25 (m, 4H); ¹³C NMR (151 MHz, DMSO) δ 163.63, 136.83, 132.81, 131.66, 128.28, 80.09 (d, J=165.1 Hz), 50.24, 47.21 (d, J=19.8 Hz).

### Synthesis of di-tert-butyl 2,2′-((((1,2,4,5-tetrazine-3,6-diyl)bis(4,1-phenylene))bis(methylene))bis((2-fluoroethyl)azanediyl))diacetate (10)

To a suspension of the hydrochloride salt of N,N′-(((1,2,4,5-tetrazine-3,6-diyl)bis(4,1-phenylene))bis(methylene))bis(2-fluoroethan-1-amine) (0.09 gr, 0.20 mmol) in anhydrous DMF (3 mL) was added Et₃N (0.13 mL, 1.00 mmol). The reaction was stirred until all the precipitate went in solution. Tert-butyl bromo acetate (0.12 mL, 0.80 mmol) was added and the reaction stirred for 6 h at 50° C. The mixture was cooled to room temperature and diluted with EtOAc (30 mL). The organic phase was washed with water (2×15 mL) and brine (2×15 mL). The organic phase was collected, dried over MgSO₄, filtered and concentrated under reduced pressure to give 0.11 gr (91%) of the desired compound as a red oil. Rf=0.48 (Heptane/EtOAc 70/30); ¹H NMR (400 MHz, CDCl₃) δ 8.62 (d, J=8.4 Hz, 4H), 7.65 (d, J=8.2 Hz, 4H), 4.64 (t, J=5.0 Hz, 2H), 4.53 (t, J=5.0 Hz, 2H), 4.04 (s, 4H), 3.42 (s, 4H), 3.14 (t, J=5.0 Hz, 2H), 3.07 (t, J=5.0 Hz, 2H), 1.51 (s, 18H); ¹³C NMR (101 MHz, CDCl₃) δ 170.58, 163.82, 144.39, 130.80, 129.63, 128.02, 83.08 (d, J=167.6 Hz), 81.19, 58.48, 55.69, 53.70 (d, J=20.0 Hz), 28.22.

### Synthesis of 2,2′-((((1,2,4,5-tetrazine-3,6-diyl)bis(4,1-phenylene))bis(methylene))bis((2-fluoroethyl)azanediyl))diacetic acid (I)

To a solution of di-tert-butyl 2,2′-((((1,2,4,5-tetrazine-3,6-diyl)bis(4,1-phenyl ene))bis(methylene))bis((2-fluoroethyl)azanediyl))diacetate (0.12 g, 0.19 mmol) in 5 mL of CH₂Cl₂ was added 2 mL of TFA. The mixture was stirred at room temperature for 4 h. The solvent was then removed under reduced pressure. Purification by preparative HPLC afforded 0.70 g (50%) of the desired compound (TFA salt) as a red solid. ¹H NMR (600 MHz, DMSO) δ 8.51 (d, J=8.2 Hz, 4H), 7.67 (d, J=7.9 Hz, 4H), 4.60 (t, J=5.0 Hz, 2H), 4.53 (t, J=5.0 Hz, 2H), 4.02 (s, 4H), 3.46 (s, 4H), 3.06 (s, 2H), 3.02 (s, 2H); ¹³C NMR (151 MHz, DMSO) δ 172.57, 163.68, 144.56, 131.23, 130.11, 128.01, 82.92 (d, J=165.3 Hz), 58.08, 54.66, 53.64 (d, J=19.8 Hz).

### Synthesis of 2-((4-(6-(4-(((2-fluoroethyl)amino)methyl)phenyl)-1,2,4,5-tetrazin-3-yl)benzyl)amino)ethan-1-ol (11)

(4-(6-(4-(((Tert-butoxycarbonyl)(2-fluoroethyl)amino)methyl)phenyl)-1,2,4,5-tetrazin-3-yl)benzyl)(2-hydroxyethyl)carbamate (0.06 gr, 0.1 mmol) was treated with a solution of HCl (4 M) in dioxane (1 mL). A precipitate was formed. Filtration afforded 0.035 gr (75%) of the desired product as hydrochloride salt. ¹H NMR (600 MHz, DMSO) δ 9.64 (s, 4H), 8.61 (d, J=8.3 Hz, 4H), 8.02-7.53 (m, 4H), 5.28 (s, 1H), 4.85 (t, J=4.6 Hz, 1H), 4.77 (t, J=4.6 Hz, 1H), 4.37 (s, 2H), 4.34 (s, 2H), 3.73 (s, 2H), 3.40 (t, J=4.8 Hz, 1H), 3.35 (t, J=4.8 Hz, 1H), 3.04 (s, 2H); ¹³C NMR (151 MHz, DMSO) δ 163.63, 137.00, 136.86, 132.81, 132.74, 131.64, 128.29, 128.27, 80.12 (d, J=165.3 Hz), 56.84, 50.27, 50.01, 49.12, 47.24 (d, J=19.9 Hz).

### Synthesis of tert-butyl N-(4-(6-(4-(((2-(tert-butoxy)-2-oxoethyl)(2-fluoroethyl)amino) methyl)phenyl)-1,2,4,5-tetrazin-3-yl)benzyl)-N-(2-hydroxyethyl)glycinate (12)

To a suspension of the hydrochloride salt of 2-((4-(6-(4-(((2-fluoroethyl)amino)methyl)phenyl)-1,2,4,5-tetrazin-3-yl)benzyl)amino)ethan-1-ol (0.03 gr, 0.077 mmol) in anhydrous DMF (3 mL) was added Et₃N (0.05 mL, 0.38 mmol). The reaction was stirred until all the precipitate went in solution. Tert-butyl bromo acetate (0.04 mL, 0.3 mmol) was added and the reaction stirred for 6 h at 50° C. The mixture was cooled to room temperature and diluted with EtOAc (30 mL). The organic phase was washed with water (2×15 mL) and brine (2×15 mL). The organic phase was collected, dried over MgSO₄, filtered and concentrated under reduced pressure to give 0.03 gr (64%) of the desired compound as a red oil. Rf=0.25 (Heptane/EtOAc 70/30); ¹H NMR (400 MHz, CDCl₃) δ 9.44-8.40 (m, 2H), 7.61 (dd, J=9.6, 8.2 Hz, 2H), 4.62 (t, J=5.0 Hz, 1H), 4.50 (t, J=5.0 Hz, 1H), 4.02 (s, 2H), 3.96 (s, 2H), 3.63 (s, 2H), 3.39 (s, 2H), 3.29 (s, 2H), 3.23 (s, 1H), 3.11 (t, J=5.0 Hz, 1H), 3.04 (t, J=5.0 Hz, 1H), 2.91 (t, J=5.1 Hz, 2H), 1.48 (s, 9H), 1.46 (s, 9H); ¹³C NMR (151 MHz, CDCl₃) δ 170.99, 170.60, 163.87, 163.75, 144.47, 143.63, 131.04, 130.76, 129.75, 129.64, 128.15, 128.05, 83.09 (d, J=167.8 Hz), 81.71, 81.20, 59.12, 58.56, 58.48, 56.99, 55.71, 55.52, 53.70 (d, J=20.1 Hz), 28.22, 28.12.

### Synthesis of tert-butyl N-(4-(6-(4-(((2-(tert-butoxy)-2-oxoethyl)(2-chloroethyl)amino) methyl)phenyl)-1,2,4,5-tetrazin-3-yl)benzyl)-N-(2-fluoroethyl)glycinate (XXXIV)

To a solution of compound tert-butyl N-(4-(6-(4-(((2-(tert-butoxy)-2-oxoethyl)(2-fluoroethyl)amino) methyl)phenyl)-1,2,4,5-tetrazin-3-yl)benzyl)-N-(2-hydroxyethyl)glycinate (0.04 g, 0.065 mmol) and DIPEA (0.034 mL, 0.19 mmol) in CH₂Cl₂ (10 mL) were added mesyl chloride (0.01 g, 0.019 mmol) and DMAP (0.001 g, 0.01 mmol). The reaction was stirred at room temperature for 12 h. The solvent was removed under reduced pressure. Purification by flash chromatography (80/20 Heptane/EtOAc) afforded 0.020 (48%) of the desired product as a red solid. Rf=0.55 (Heptane/EtOAc 70/30); ¹H NMR (400 MHz, CDCl₃) δ 8.60 (d, J=8.1 Hz, 4H), 7.62 (dd, J=8.4, 3.0 Hz, 4H), 4.62 (t, J=5.0 Hz, 1H), 4.50 (t, J=5.0 Hz, 1H), 4.11-3.89 (m, 4H), 3.57 (t, J=6.8 Hz, 2H), 3.39 (s, 2H), 3.36 (s, 2H), 3.15-3.10 (m, 3H), 3.04 (t, J=5.0 Hz, 1H), 1.48 (s, 18H); ¹³C NMR (101 MHz, CDCl₃) δ 170.51, 163.84, 163.81, 130.87, 130.79, 129.64, 129.56, 128.05, 128.03, 83.08 (d, J=167.8 Hz), 81.35, 81.19, 58.48, 58.12, 55.96, 55.70, 55.50, 53.70 (d, J=20.0 Hz), 28.23.

### Synthesis of di-tert-butyl 2,2′-((((1,2,4,5-tetrazine-3,6-diyl)bis(4,1-phenylene)) bis(methylene))bis((2-hydroxyethyl)azanediyl))diacetate (13)

To a suspension of tert-butyl (4-cyanobenzyl)(2-hydroxyethyl)carbamate (4 gr, 14.47 mmol) and Zn(OTf)₂ (2.63 gr, 7.23 mmol) in EtOH (40 mL) was added hydrazine monohydrate (14.04 mL, 289 mmol). The mixture was allowed to stir at 70° C. for 22 hours, and when the reaction is completed, is cooled at room temperature. The volatiles were removed under reduced pressure and the residue solubilized in EtOH (80 mL). A solution of NaNO₂ (19.97 g, 289.00 mmol) in water (50 mL) was added to the crude reaction followed by dropwise addition of HCl (2M) until gas evolution ceased and a pH of 2-3 was achieved producing a red mixture. The crude reaction was extracted with DCM (3×60 mL) and washed with brine (3×50 mL). The organic phase was collected, dried over MgSO₄, filtered and concentrated under reduced pressure. Purification by flash chromatography (DCM/MeOH 95/5) afforded 1.2 g (28%) of the desired product as a red solid (mixture of rotamers). Rf=0.21 (DCM/MeOH 95/5); ¹H NMR (600 MHz, CDCl₃) δ 8.63 (d, J=7.9 Hz, 4H), 7.49 (d, J=7.9 Hz, 4H), 4.62 (s, 4H), 3.79 (s, 4H), 3.60-3.37 (m, 4H), 3.01 (s, 2H), 1.48 (s, 18H); ¹³C NMR (151 MHz, CDCl₃) δ 163.74, 157.21, 156.07, 143.55, 130.84, 128.25, 127.88, 80.91, 62.19, 61.44, 52.12, 51.13, 50.28, 49.41, 28.39.

### Synthesis of di-tert-butyl 2,2′-((((1,2,4,5-tetrazine-3,6-diyl)bis(4,1-phenylene)) bis(methylene))bis((2-hydroxyethyl)azanediyl))diacetate (14)

(4-(6-(4-(((Tert-butoxycarbonyl)(2-fluoroethyl)amino)methyl)phenyl)-1,2,4,5-tetrazin-3-yl)benzyl)(2-hydroxyethyl)carbamate (0.9 gr, 1.55 mmol) was treated with a solution of HCl (4 M) in dioxane (15 mL). A precipitate was formed. Filtration afforded 0.68 gr (97%) of the desired product as hydrochloride salt. ¹H NMR (400 MHz, DMSO) δ 9.41 (s, 4H), 8.59 (d, J=8.4 Hz, 4H), 7.89 (d, J=8.4 Hz, 4H), 5.29 (s, 2H), 4.33 (s, 4H), 3.74 (s, 4H), 3.04 (s, 4H); ¹³C NMR (101 MHz, DMSO) δ 163.62, 137.03, 132.71, 131.65, 128.23, 56.84, 49.98, 49.14.

### Synthesis of di-tert-butyl 2,2′-((((1,2,4,5-tetrazine-3,6-diyl)bis(4,1-phenylene))bis(methylene))bis((2-hydroxyethyl)azanediyl))diacetate (15)

To a suspension of the hydrochloride salt of di-tert-butyl 2,2′-((((1,2,4,5-tetrazine-3,6-diyl)bis(4,1-phenylene)) bis(methylene))bis((2-hydroxyethyl)azanediyl))diacetate (0.25 gr, 0.55 mmol) in anhydrous DMF (10 mL) was added Et₃N (0.38 mL, 2.75 mmol). The reaction was stirred until all the precipitate went in solution. Tert-butyl bromo acetate (0.32 mL, 2.20 mmol) was added and the reaction stirred for 6 h at 50° C. The mixture was cooled to room temperature and diluted with EtOAc (40 mL). The organic phase was washed with water (2×15 mL) and brine (2×15 mL). The organic phase was collected, dried over MgSO₄, filtered and concentrated under reduced pressure to give 0.31 gr (91%) of the desired compound as a red oil. Rf=0.23 (Heptane/EtOAc 60/40); ¹H NMR (400 MHz, CDCl₃) δ 8.28 (d, J=8.0 Hz, 4H), 7.27 (d, J=8.0 Hz, 4H), 3.62 (s, 4H), 3.31 (t, J=5.2 Hz, 4H), 2.96 (s, 4H), 2.58 (t, J=5.1 Hz, 4H), 1.14 (s, 18H); ¹³C NMR (101 MHz, CDCl₃) δ 171.14, 163.75, 143.96, 130.90, 129.66, 128.11, 81.56, 59.21, 58.57, 56.95, 55.67.

### Synthesis of di-tert-butyl 2,2′-((((1,2,4,5-tetrazine-3,6-diyl)bis(4,1-phenylene))bis(methylene))bis((2-fluoroethyl)azanediyl))diacetate (10) and di-tert-butyl 2,2′-((((1,2,4,5-tetrazine-3,6-diyl)bis(4,1-phenylene))bis(methylene))bis((2-hydroxyethyl)azanediyl))diacetate (12)

To a solution of di-tert-butyl 2,2′-((((1,2,4,5-tetrazine-3,6-diyl)bis(4,1-phenylene))bis(methylene))bis((2-hydroxyethyl)azanediyl))diacetate (0.24 g, 0.39 mmol) in anhydrous THF (15 mL) at −78° C. was added DAST (0.055 mL, 0.39 mmol). The resulting mixture was stirred for 1 hour at −78° C. and additional 3 hours at room temperature. Subsequently the reaction was quenched with NaHCO₃ saturated solution (10 mL) and stirred for 30 minutes. The reaction mixture was extracted with DCM (3×30 mL) and washed with brine (3×30 mL). The organic phase was collected, dried over MgSO₄, filtered and concentrated under reduced pressure. Purification by flash chromatography (DCM/MeOH 95/5) afforded 0.12 g (50%) of di-tert-butyl 2,2′-((((1,2,4,5-tetrazine-3,6-diyl)bis(4,1-phenylene))bis(methylene))bis((2-fluoroethyl)azanediyl))diacetate (NMR see example x). A more polar fraction corresponding to di-tert-butyl 2,2′-((((1,2,4,5-tetrazine-3,6-diyl)bis(4,1-phenylene))bis(methylene))bis((2-hydroxyethyl)azanediyl))diacetate was isolated in 0.04 g (14%).

### Example 2

Synthesis of Unsymmetrical Tetrazines and their Precursors

Compounds XII, XIII, and their precursors XLVIII, L are obtained as disclosed in paper Garcia-Vázquez, R.; Battisti, U. M.; JØrgensen, J. T.; Shalgunov, V.; Hvass, L.; Stares, D. L.; Petersen, I. N.; Crestey, F. C.; Ldffler, A.; Svatunek, D.; et al. Direct Cu-mediated aromatic 18F-labeling of highly reactive tetrazines for pretargeted bioorthogonal PET imaging. Chem. Sci. 2021, 12, 11668-11675 and Battisti, U. M.; Bratteby, K.; JØrgensen, J. T.; Hvass, L.; Shalgunov, V.; Mikula, H.; Kjwr, A.; Herth, M. M. Development of the First Aliphatic 18F-Labeled Tetrazine Suitable for Pretargeted PET Imaging-Expanding the Bioorthogonal Tool Box. J. Med. Chem. 2021, 64, 15297-15312

### Example 2.1

### Compound XLVIII

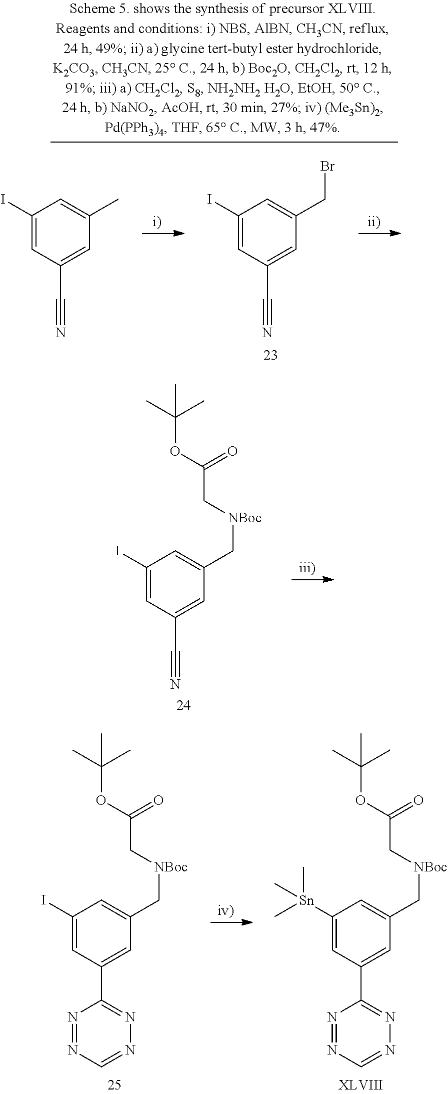

### 3-(Bromomethyl)-5-iodobenzonitrile (23)

To a solution of 3-iodo-5-methylbenzonitrile (2.50 g, 10.28 mmol) and N-bromosuccinimide (2.28 g, 12.86 mmol) in CH₃CN (40 mL) was added AIBN (0.67 g, 4.11 mmol). The reaction was refluxed for 24 h. The solvent was removed under vacuum and the crude purified by flash chromatography (95/5 n-heptane/EtOAc) to give 1.61 g (49%) of desired product as a white solid. Rf=0.28 (95/5 n-heptane/EtOAc); ¹H NMR (400 MHz, CDCl₃) δ 7.96 (d, J=1.6 Hz, 1H), 7.89 (d, J=1.6 Hz, 1H), 7.64 (t, J=1.6 Hz, 1H), 4.38 (s, 2H)¹³C NMR (101 MHz, CDCl₃) δ 142.20, 140.90, 140.12, 131.67, 116.55, 114.56, 94.05, 29.90.

### tert-Butyl 2-((tert-butoxycarbonyl)(3-cyano-5-iodobenzyl)amino)acetate (24)

To a solution of 3-(bromomethyl)-5-iodobenzonitrile (2.00 g, 6.21 mmol) in CH₃CN (40 mL) was added K₂CO₃ (4.29 g, 31.07 mmol) and glycine tert-butyl ester hydrochloride (3.12 g, 18.63 mmol). The reaction mixture was stirred at rt overnight. The solvent was removed under reduced pressure, and the resulting mixture was diluted with water (20 mL), extracted with EtOAc (2×25 mL), washed with brine (30 mL), dried over MgSO₄, filtered and concentrated. Purification by flash chromatography (80/20 n-heptane/EtOAC); afforded 1.91 g (83%) of the desired product as a colorless oil. Rf=0.23 (80/20 n-heptane/EtOAC); 1H NMR (400 MHz, CDCl₃) δ 7.89 (s, 1H), 7.79 (s, 1H), 7.56 (s, 1H), 3.72 (s, 2H), 3.21 (s, 2H), 1.91 (s, 1H), 1.41 (s, 9H); 13C NMR (101 MHz, CDCl₃) δ 171.24, 143.30, 141.56, 139.06, 130.89, 117.13, 114.11, 93.95, 81.68, 51.74, 50.76, 28.13.

To a solution of this amine (1.9 g, 5.10 mmol) and Et₃N (1.71 mL, 12.25 mmol) in CH₂Cl₂ (40 mL) was added Boc₂O (1.41 g, 6.12 mmol). The reaction was stirred at room temperature for 12 hours. The solution was then washed with water (50 mL) and K₂CO₃ saturated solution (50 mL), dried over anhydrous Na₂SO₄, filtered and concentrated under reduced pressure to afford 2.5 g of the crude. Purification by flash chromatography (80/20 n-heptane/EtOAc) afforded 2.2 g (91%) of the desired product as a colorless oil (60/40 unassigned rotamers mixture). Rf=0.38 (80/20 n-heptane/EtOAc); ¹H NMR (400 MHz, CDCl₃) δ 7.99-7.80 (m, 2H), 7.71-7.41 (m, 1H), 4.48 (s, 1.2H), 4.42 (s, 0.8H), 3.86 (s, 0.8H), 3.70 (s, 1.2H), 1.51-1.42 (m, 18H); ¹³C NMR (101 MHz, CDCl₃) δ 168.43, 155.62, 142.14, 141.49, 141.13, 140.79, 139.30, 131.33, 130.50, 130.04, 117.00, 114.29, 94.03, 82.03, 81.93, 81.30, 81.13, 50.85, 50.56, 49.78, 49.53, 28.22, 28.05.

### tert-Butyl 2-((tert-butoxycarbonyl)(3-iodo-5-(1,2,4,5-tetrazin-3yl)benzyl)amino) acetate (25)

CH₂Cl₂ (0.17 mL, 2.67 mmol), sulfur (0.17 g, 0.67 mmol), hydrazine monohydrate (1.1 mL, 21.39 mmol) and ethanol (4.0 mL) along with tert-butyl 2-((tert-butoxycarbonyl)(3-cyano-5-iodobenzyl)amino)acetate (2.10 g, 4.44 mmol) were added to a Biotage microwave vial (10-20 mL) equipped with a stir bar. The vessel was sealed, and the reaction mixture was heated to 50° C. for 24 hours, before being allowed to cool to room temperature and unsealed. Then 3 ml of CH₂Cl₂ and NaNO₂ (1.84 g, 26.73 mmol) in water (30 ml) were added to the now yellow mixture followed by dropwise addition of acetic acid (10 mL), producing a mixture red in color. The reaction mixture was extracted with CH₂Cl₂, washed with brine, dried with MgSO₄ and filtered before concentrating in vacuo. The crude was purified using flash chromatography (95/5 n-heptane/EtOAc) to yield 0.64 g (27%) of the desired product as a red solid (60/40 unassigned rotamers mixture). Rf=0.31 (80/20 n-heptane/EtOAc); ¹H NMR (400 MHz, CDC₃) δ 10.18 (s, 1H), 8.84-8.71 (m, 1H), 8.45-8.36 (m, 1H), 7.87-7.74 (m, 1H), 4.54 (s, 1.2H), 4.47 (s, 0.8H), 3.85 (s, 0.8H), 3.69 (s, 1.2H), 1.44-1.36 (m, 9H), 1.38 (s, 9H); ¹³C NMR (101 MHz, CDC₃) δ 168.63, 165.14, 158.04, 155.71, 155.39, 141.70, 141.46, 141.26, 140.93, 136.06, 135.93, 133.58, 133.52, 126.70, 126.48, 95.21, 81.83, 81.74, 81.07, 80.85, 51.11, 50.71, 49.47, 49.33, 28.31, 28.26, 28.07.

### tert-Butyl N-(3-(1,2,4,5-tetrazin-3-yl)-5-(trimethylstannyl)benzyl)-N-(tert-butoxycarbonyl)glycinate (XLVIII)

Pd(PPh₃)₄ (19.4 mg, 10%) and hexamethylditin (87 μL, 0.42 mmol) were successively added to a microwave vial equipped with a stir bar which was then sealed and purged with N₂. A solution of tert-butyl 2-((tert-butoxycarbonyl)(3-iodo-5-(1,2,4,5-tetrazin-3yl)benzyl)amino) acetate (0.08 g, 0.17 mmol) in dry THF (2.5 mL) was added via a syringe and the reaction allowed to stir at 65° C. in a microwave for 3 hours. The reaction was allowed to cool to room temperature and unsealed before being quenched with saturated aqueous KF (1 mL). The solution was extracted with CH₂Cl₂ (3×10 mL) washed with brine (10 mL), dried over MgSO₄, filtered and concentrated under reduced pressure. The crude was purified using flash chromatography (95/5 n-heptane/EtOAc) to yield 0.055 g (66%) of the desired product as a purple oil (60/40 unassigned rotamers mixture). Rf=0.38 (80/20 n-neptane/EtOAc); ¹H NMR (400 MHz, CDC₃) δ 10.21 (s, 1H), 8.74-8.60 (m, 1H), 8.45-8.37 (m, 1H), 7.70-7.61 (m, 2H), 4.65 (s, 1.2H), 4.58 (s, 0.8H), 3.90 (s, 0.8H), 3.75 (s, 1.2H), 1.49 (s, 9H), 1.44 (s, 9H), 0.36 (s, 9H); ¹³C NMR (101 MHz, Chloroform-d) δ 168.85, 166.70, 157.76, 155.81, 155.62, 144.69, 140.25, 139.75, 138.58, 138.33, 134.55, 131.09, 127.41, 81.62, 81.51, 80.69, 80.51, 51.60, 51.17, 49.25, 48.95, 28.37, 28.29, 28.07, −9.34.

### Example 3

### Synthesis of Isomer Free TCO

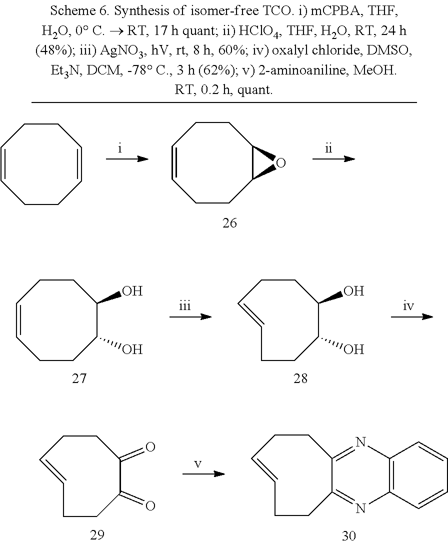

(Z)-9-Oxabicyclo[6.1.0]non-4-ene (26). Cylcooctadiene (1.00 g, 9.24 mmol) was dissolved in dry DCM (20 mL) and cooled to 0° C., to which was added mCBPA (2.07 g, 9.24 mmol) in a portion wise manner. After addition, the reaction was stirred for 30 min at 0° C. and afterwards the reaction was allowed to warm to room temperature. The reaction was stirred for 17 h. The reaction was quenched with 2M K₂CO₃ (20 mL) and the crude was extracted with 3×DCM (15 mL). The combined organic phases was washed with K₂CO₃ (25 mL), and brine (25 mL). The solvent was evaporated, and the crude was without purification used in the next reaction.

(Z)-Cyclooct-5-ene-1,2-diol (27). 25 (1.15 g, 9.24 mmol) was dissolved in THF/water (3:2 v/v, 50 mL) mixture containing HClO₄ (250 μL, 2.90 mmol). The reaction was stirred at room temperature for 24 h. Water (10 mL) was added to the reaction mixture, and the product was extracted with 3×Et₂O (20 mL). The combined ether fractions were washed with 2×2M NaHCO₃ (25 mL). The crude was purified by CombiFlash, which yielded a clear viscous oil. (624 mg, 4.40 mmol, 48%). ¹H NMR (600 MHz, Chloroform-d) δ 5.64-5.56 (m, 2H), 3.72-3.65 (m, 2H), 2.41-2.33 (m, 2H), 2.17-2.09 (m, 4H), 1.63-1.55 (m, 2H); ¹³C NMR (151 MHz, Chloroform-d) δ 129.3, 74.1, 33.6, 22.9.

### (E)-Cyclooct-5-ene-1,2-diol (28)

The compound was obtained as described in Royzen, M.; Yap, G. P. A.; Fox, J. M. A Photochemical Synthesis of Functionalized trans-Cyclooctenes Driven by Metal Complexation. Journal of the American Chemical Society 2008, 130, 3760-3761. A flash cartridge (220 g, screw top, luer lock end fittings, Cat #FCSTLL-220-6) was packed with 8 cm silica (15-40 μm) on the bottom and silver nitrate impregnated silica until the top. The column was flushed with 9:1 diethyl ether/n-heptane (500 mL) and the column was protected from light with aluminium foil. The cooling fence and UV lamps were turned on and after 10 minutes no detection of silver leakage was observed. Methyl benzoate (1 mL), 27 (1 g) and an additional 50 mL 9:1 diethyl ether/n-heptane solution were added to a round-bottom flask. The mixture was then added to the quartz flask. The pump was turned on (flowrate=100 mL/min) and the photoreactor was turned on and photoisomerization was conducted for 8 hours. After 8 hours, the photoreactor was turned off and the column was dried by a stream of air. The silica was removed from the column and washed with 400 mL ammonia and 400 mL DCM. The mixture was stirred for 30 minutes, filtered and the organic layer was collected. The organic layer was washed with brine, dried with MgSO4, filtered and concentrated to give 0.60 g (60%) of the product as a yellowish oil (mixture of axial and equatorial isomers); ¹H NMR (CDCl₃, 400 MHz) δ 5.45-5.36 (m, 2H), 3.58 (bs, 2H), 3.47 (m, 2H), 2.35-2.21 (m, 4H), 2.02-1.98 (m, 2H), 1.76-1.65 (m, 2H). ¹³C NMR (CDCl3, 100 MHz) δ 132.6, 76.0, 40.7, 32.6.

### (E)-Cyclooct-5-ene-1,2-dione (29)

Under Argon atmosphere, at −78° C., a solution of DMSO (1.05 mL, 14.77 mmol) in dry DCM (30 mL) was added drop wise to a solution of (COCl)₂ (736 μL, 8.44 mmol) in dry DCM (15 mL). After addition, the reaction mixture is stirred for 30 min at −78° C. A solution of 28 (500 mg, 3.52 mmol) in dry DCM (10 mL) is added drop wise and the reaction mixture is stirred an additional 30 minutes at −78° C. Et₃N (4.90 mL, 35.16 mmol) is added dropwise and the reaction mixture was kept at −78° C. for an additional 60 minutes. The reaction mixture was allowed to warm to room temperature and was stirred an additional hour. The reaction mixture is washed with water (50 mL), 2×0.5 M HCl_{(aq)} (50 mL) and brine (30 mL). The organic phase is dried over MgSO₄ and evaporated, which yielded the desired compound as a clear liquid. (302 mg, 2.19 mmol, 62%).

### (E)-6,7,10,11-Tetrahydrocycloocta[b]quinoxaline (30)

The compound was obtained following the procedure reported in Delpivo, C.; Micheletti, G.; Boga, C. A Green Synthesis of Quinoxalines and 2,3-Dihydropyrazines. Synthesis 2013, 45, 1546-1552. A 50 mL round-bottomed flask was charged with a solution of the appropriate 29 (138 mg, 1 mmol) in MeOH (3 mL). To this solution was added the respective 2-aminoaniline (110 mg, 1 mmol) and the mixture was stirred at r.t. After 30 min, the crude mixture was extracted with CH₂Cl₂ (3×10 mL). The combined organic layers were dried (MgSO4) and after filtration, the solvent was removed to give 0.2 g (97%) the desired product. ¹H NMR (400 MHz, CDCl₃) δ 8.23-7.93 (m, 2H), 7.77-7.55 (m, 2H), 6.19-5.93 (m, 1H), 5.24-5.09 (m, 1H), 3.40-3.23 (m, 2H), 3.22-3.12 (m, 1H), 3.02 (td, J=12.8, 2.8 Hz, 1H), 2.80-2.69 (m, 1H), 2.69-2.54 (m, 1H), 2.50-2.38 (m, 1H), 2.36-2.25 (m, 1H).

### Example 3.1

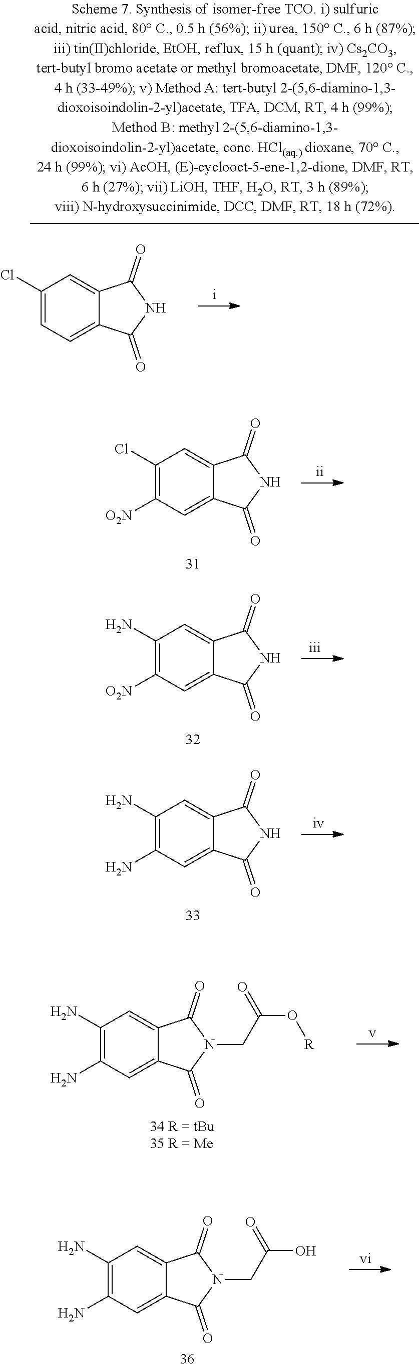

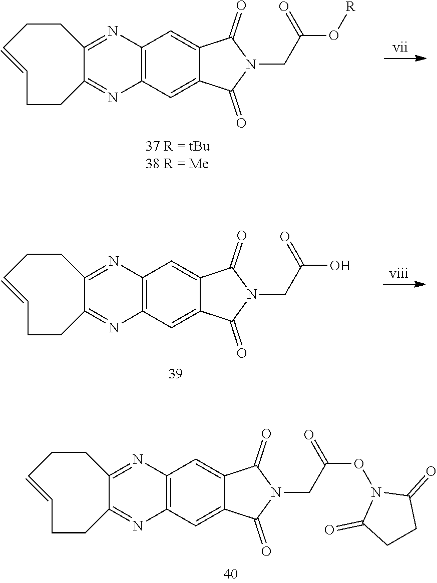

### 5-Chloro-6-nitroisoindoline-1,3-dione (31)

The compound was synthesized according to the literature.1 A mixture of concentrated sulfuric acid (33.1 mL) and fuming nitric acid (2.26 mL) was added dropwise to 4-chloro-phthalimide (5.00 g, 27.53 mmol). The reaction mixture was heated at 80° C. for 0.5 h. After cooling to r.t. the dark red solution was poured into ice. The resulted yellow precipitate was collected by filtration, washed with water, dried in vacuum affording a pale-yellow solid that was recrystallized from EtOH to give 3.5 g (56% yield) of the desired product. Rf=0.45 (n-Heptane/EtOAc 60/40); ¹H NMR (400 MHz, DMSO) δ 11.87 (s, 1H), 8.51 (s, 1H), 8.27 (s, 1H); ¹³C NMR (101 MHz, DMSO) δ 167.21, 167.17, 151.86, 136.54, 133.00, 130.89, 126.60, 120.20.

### 5-Amino-6-nitroisoindoline-1,3-dione (32)

The compound was synthesized according to the literature.1 A mixture of 5-chloro-6-nitroisoindoline-1,3-dione (2.4 g, 10.59) and urea (6.36 g, 105.92 mmol) was stirred and heated to 150° C. under argon for 6 h. After cooled to rt, the solid was suspended in hot water (80° C.), filtered, washed with hot water (3×50 mL). The solid was recrystallized from EtOH to give 1.92 g (87%) of a yellow solid. Rf=0.28 (n-Heptane/EtOAc 60/40); ¹H NMR (600 MHz, DMSO) δ 11.43 (s, 1H), 8.56-8.05 (m, 3H), 7.40 (s, 1H); ¹³C NMR (151 MHz, DMSO) δ 168.01, 150.64, 138.24, 132.50, 122.38, 117.74, 114.65.

### 5,6-Diaminoisoindoline-1,3-dione (33)

The compound was synthesized according to the literature.1 To a suspension of 4-amino-5-nitro-phthalimide (1.0 g, 4.27 mmol) and Tin(II) chloride (4.57 g, 24.14 mmol) in ethanol (30 mL) was refluxed under argon for 15 h. The resulted bright red suspension was cooled to r.t., the red solid was collected by filtration, washed with ethanol and dried in vacuum to give a red orange solid. Rf=0.15 (95/5 DCM/MeOH); ¹H NMR (400 MHz, DMSO) δ 10.25 (s, 1H), 6.81 (s, 2H), 5.50 (s, 4H); ¹³C NMR (101 MHz, DMSO) δ 170.72, 140.30, 122.83, 107.03.

### tert-Butyl 2-(5,6-diamino-1,3-dioxoisoindolin-2-yl)acetate (34)

To a suspension of Cs₂CO₃ (1.93 g, 5.92 mmol) and 5,6-diaminoisoindoline-1,3-dione (1.0 g, 5.64 mmol) in anhydrous DMF (5 mL) was added tert-butyl bromoacetate (0.87 mL, 5.92 mmol). The reaction was heated at 120° C. for 4 h under argon. The mixture was cooled to r.t. and water (20 mL) was added. The reaction mixture was extracted with DCM (3×50 mL). The organic phase was dried over anhydrous MgSO4, filtered and concentrated under reduced pressure. The resulting residue was purified using flash chromatography (98/2 DCM/MeOH) to yield 0.8 g (49%) of the desired compound as a yellow solid. Rf=0.42 (DCM/MeOH 93/7); ¹H NMR (600 MHz, DMSO) δ 6.89 (s, 2H), 5.61 (s, 4H), 4.11 (s, 2H), 1.40 (s, 9H); ¹³C NMR (151 MHz, DMSO) δ 168.63, 167.62, 140.46, 121.55, 107.34, 82.08, 40.55, 28.08.

### Methyl 2-(5,6-diamino-1,3-dioxoisoindolin-2-yl)acetate (35)

To a suspension of Cs₂CO₃ (1.93 g, 5.92 mmol) and 5,6-diaminoisoindoline-1,3-dione (1.0 g, 5.64 mmol) in anhydrous DMF (5 mL) was added methyl bromoacetate (0.63 mL, 5.92 mmol). The reaction was heated at 120° C. for 4 h under argon. The mixture was cooled to r.t. and water (20 mL) was added. The reaction mixture was extracted with DCM (3×50 mL). The organic phase was dried over anhydrous MgSO4, filtered and concentrated under reduced pressure. The resulting residue was purified using flash chromatography (98/2 DCM/MeOH) to yield 0.45 g (33%) of the desired compound as a yellow solid. Rf=0.40 (DCM/MeOH 93/7); ¹H NMR (400 MHz, DMSO) δ 6.90 (s, 2H), 5.63 (s, 4H), 4.25 (s, 2H), 3.67 (s, 3H); ¹³C NMR (101 MHz, DMSO) δ 169.13, 168.50, 140.52, 121.47, 107.37, 52.72, 38.69.

### 2-(5,6-diamino-1,3-dioxoisoindolin-2-yl)acetic acid (36)Method A

To a solution of tert-butyl 2-(5,6-diamino-1,3-dioxoisoindolin-2-yl)acetate (0.4 g, 1.37 mmol) in DCM (6 mL) was added TFA (2 mL). The reaction was stirred at r.t. for 4 h and then concentrated under reduced pressure to give 0.32 g (99%) of the desired compound as a yellow solid. Rf=0.29 (DCM/MeOH 99/1+0.1% AcOH); ¹H NMR (400 MHz, DMSO) δ 6.90 (s, 2H), 4.13 (s, 2H); ¹³C NMR (101 MHz, DMSO) δ 169.95, 168.60, 140.16, 121.80, 107.71, 38.86.

### Method B

To a solution of methyl 2-(5,6-diamino-1,3-dioxoisoindolin-2-yl)acetate (0.25 g, 1.01 mmol) in dioxane (6 mL) was added conc. HCl (1 mL). The reaction was stirred at 70° C. for 24 h and then concentrated under reduced pressure to give 0.23 g (99%) of the desired compound as a yellow solid. Rf=0.29 (DCM/MeOH 99/1+0.1% AcOH); ¹H NMR (400 MHz, DMSO) δ 6.90 (s, 2H), 4.13 (s, 2H); 13C NMR (101 MHz, DMSO) δ 169.95, 168.60, 140.16, 121.80, 107.71, 38.86.

### tert-Butyl (E)-2-(1,3-dioxo-1,3,6,7,10,11-hexahydro-2H-cycloocta[b]pyrrolo [3,4-g]quinoxalin-2-yl)acetate (37)

A solution of tert-butyl 2-(5,6-diamino-1,3-dioxoisoindolin-2-yl)acetate (0.4 g, 1.40 mmol) and acetic acid (0.8 mL, 14.03 mmol) in DMF (5 mL) and water (1 mL) was added to (E)-cyclooct-5-ene-1,2-dione (0.21 g, 1.54 mmol). The reaction was stirred at r.t. under dark for 6 h. The solution was diluted with 10 mL of water and purified directly by preparative HPLC to give 0.09 g (17%) of the desired compound as a yellow solid. Rf=0.36 (Heptane/EtOAc 75/25); ¹H NMR (400 MHz, CDCl₃) δ 8.61-8.37 (m, 2H), 6.10 (ddd, J=15.8, 10.7, 3.9 Hz, 1H), 5.15 (ddd, J=16.3, 9.5, 5.8 Hz, 1H), 4.42 (s, 2H), 3.40-3.28 (m, 2H), 3.24 (dd, J=10.9, 5.1 Hz, 1H), 3.08 (td, J=12.8, 2.8 Hz, 1H), 2.85-2.66 (m, 2H), 2.45 (q, J=11.9 Hz, 1H), 2.39-2.24 (m, 1H), 1.48 (s, 9H).

### Methyl (E)-2-(1,3-dioxo-1,3,6,7,10,11-hexahydro-2H-cycloocta[b]pyrrolo [3,4-g]quinoxalin-2-yl)acetate (38)

A solution of methyl 2-(5,6-diamino-1,3-dioxoisoindolin-2-yl)acetate (0.38 g, 1.40 mmol) and acetic acid (0.8 mL, 14.03 mmol) in DMF (5 mL) and water (1 mL) was added to (E)-cyclooct-5-ene-1,2-dione (0.21 g, 1.54 mmol). The reaction was stirred at r.t. under dark for 6 h. The solution was diluted with 10 mL of water and purified directly by preparative HPLC to give 0.15 g (26%) of the desired compound as a yellow solid. Rf=0.31 (Heptane/EtOAc 75/25); ¹H NMR (600 MHz, CDCl₃) δ 8.53-8.46 (m, 2H), 6.10 (ddd, J=15.7, 10.8, 3.9 Hz, 1H), 5.14 (ddd, J=16.2, 9.5, 5.8 Hz, 1H), 4.53 (s, 2H), 3.79 (s, 3H), 3.34 (ddt, J=14.9, 11.1, 5.4 Hz, 2H), 3.24 (dd, J=11.2, 5.1 Hz, 1H), 3.08 (td, J=12.8, 2.8 Hz, 1H), 2.84-2.65 (m, 2H), 2.50-2.39 (m, 1H), 2.33 (ddd, J=11.1, 9.3, 5.5 Hz, 1H).

### (E)-2-(1,3-Dioxo-1,3,6,7,10,11-hexahydro-2H-cycloocta[b]pyrrolo [3,4-g]quinoxalin-2-yl)acetic acid (39)

A solution of 2-(5,6-diamino-1,3-dioxoisoindolin-2-yl)acetic acid (0.33 g, 1.40 mmol) and acetic acid (0.8 mL, 14.03 mmol) in DMF (5 mL) and water (1 mL) was added to (E)-cyclooct-5-ene-1,2-dione (0.21 g, 1.54 mmol). The reaction was stirred at r.t. under dark for 6 h. The solution was diluted with 10 mL of water and purified directly by preparative HPLC to give 0.13 g (27%) of the desired compound as a yellow solid. Rf=0.45 (DCM/MeOH 99/1+0.1% AcOH); ¹H NMR (600 MHz, DMSO) δ 13.33 (s, 1H), 8.47 (d, J=9.4 Hz, 2H), 6.18 (ddd, J=17.2, 10.7, 3.8 Hz, 1H), 5.17-5.08 (m, 1H), 4.42 (s, 2H), 3.52-3.43 (m, 1H), 3.23 (dd, J=9.2, 3.1 Hz, 2H), 3.12 (dd, J=11.2, 5.1 Hz, 1H), 2.71 (tt, J=17.0, 6.0 Hz, 2H), 2.32 (qd, J=11.0, 7.6 Hz, 1H), 2.17 (tt, J=11.3, 5.4 Hz, 1H); ¹³C NMR (151 MHz, DMSO) δ 169.10, 166.50, 161.93, 161.59, 143.21, 143.07, 137.16, 132.73, 130.81, 130.63, 125.34, 125.12, 46.31, 41.35, 34.30, 28.33.

### 2,5-Dioxopyrrolidin-1-yl (E)-2-(1,3-dioxo-1,3,6,7,10,11-hexahydro-2H-cycloocta[b]pyrrolo[3,4-g]quinoxalin-2-yl)acetate (40)

To a solution of (E)-2-(1,3-dioxo-1,3,6,7,10,11-hexahydro-2H-cycloocta[b]pyrrolo [3,4-g]quinoxalin-2-yl)acetic acid (0.06 g, 0.18 mmol) in anhdrous DMF (3 mL) under argon was added DCC (0.04 g, 0.19 mmol) and then N-hydroxysuccinimide (0.04 g, 0.35 mmol). The reaction was stirred overnight at r.t. under argon. EtOAc (10 mL) was added, and the organic layer washed with water (2×10 mL) and brine (2×10 mL). The organic phase was dried over anhydrous MgSO4, filtered and concentrated under reduced pressure. The residue was solubilized in dry cold MECN (4 mL), filtered and concentrated to give a yellow solid. The solid was triturated in diethyl ether and filtered to give 0.056 g (72%) of the desired compound as a yellow solid. Rf=0.41 (n-Heptane/EtOAc 50/50); ¹H NMR (400 MHz, CDCl₃) δ 8.56-8.47 (m, 2H), 6.10 (td, J=12.1, 5.9 Hz, 1H), 5.25-5.07 (m, 1H), 4.87 (s, 2H), 3.40-3.30 (m, 2H), 3.30-3.21 (m, 1H), 3.15-3.04 (m, 1H), 2.92-2.68 (m, 6H), 2.45 (q, J=11.9 Hz, 1H), 2.33 (dt, J=12.1, 6.1 Hz, 1H); ¹³C NMR (101 MHz, CDCl₃) δ 168.16, 165.58, 163.15, 161.67, 161.31, 143.40, 143.24, 136.54, 132.67, 130.16, 130.00, 126.26, 126.07, 46.74, 41.81, 37.05, 34.19, 28.26, 25.55.

### Example 3.2

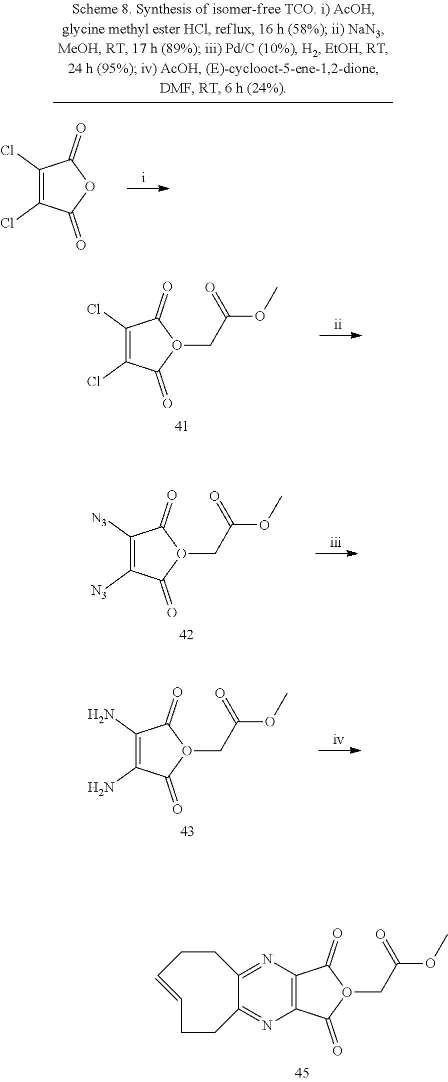

### Methyl 2-(3,4-dichloro-2,5-dioxo-2,5-dihydro-1H-pyrrol-1-yl)acetate (41)

To a solution of dichloromaleic anhydride (5.0 g, 29.95 mmol) in acetic acid (100 mL) was added glycine methyl ester hydrochloride (4.51 g, 35.94 mmol). The mixture was heated at reflux during 16 h. The solvent was removed under reduced pressure. Water (100 mL) was added, and the resulting suspension was stirred for 1 h. The solid was filtered to give 4.15 g (58%) of the desired product as a beige solid. ¹H NMR (400 MHz, DMSO) δ 4.41 (s, 2H), 3.70 (s, 3H); ¹³C NMR (101 MHz, DMSO) δ 167.96, 162.82, 133.39, 53.09, 40.13.

### Methyl 2-(3,4-diazido-2,5-dioxo-2,5-dihydro-1H-pyrrol-1-yl)acetate (42)

A solution of 2-(3,4-dichloro-2,5-dioxo-2,5-dihydro-1H-pyrrol-1-yl)acetate (4.15 g, 17.43 mmol) and sodium azide (2.72 g, 41.84 mmol) in methanol (70 mL) was stirred overnight. The solvent was removed under vacuum, the residue dissolved in dichloromethane (50 mL), washed with water (2×30 mL) and brine (2×30 mL), dried over anhydrous sodium sulphate, and the solvent removed by vacuum to give the compound (3.89 g, 89%) as a yellow solid. ¹H NMR (400 MHz, DMSO) δ 4.34 (s, 2H), 3.70 (s, 3H); ¹³C NMR (101 MHz, DMSO) δ 168.14, 164.29, 120.16, 53.03, 39.25.

### Methyl 2-(3,4-diazido-2,5-dioxo-2,5-dihydro-1H-pyrrol-1-yl)acetate (43)

To solution of the 2-(3,4-diazido-2,5-dioxo-2,5-dihydro-1H-pyrrol-1-yl)acetate (1.6 g, 6.37 mmol) in EtOH (40 mL) was added 10% Pd/C (0.34 g, 0.31 mmol). The mixture was stirred at rt and put under H₂ atmosphere for 24 h. The catalyst was then filtered on a celite pad and the solvent removed to give 1.2 g (95%) of the desired compound as a red oil. NMR ok. ¹H NMR (400 MHz, DMSO) δ 4.94 (s, 4H), 4.09 (s, 2H), 3.65 (s, 4H); ¹³C NMR (101 MHz, DMSO) δ 169.38, 169.16, 118.89, 52.63, 38.46.

### Methyl (E)-2-(1,3-dioxo-1,3,5,6,9,10-hexahydro-2H-cycloocta[b]pyrrolo[3,4-e]pyrazin-2-yl)acetate (45)

A solution of methyl 2-(3,4-diazido-2,5-dioxo-2,5-dihydro-1H-pyrrol-1-yl)acetate (0.28 g, 1.40 mmol) and acetic acid (0.8 mL, 14.03 mmol) in DMF (5 mL) and water (1 mL) was added to (E)-cyclooct-5-ene-1,2-dione (0.21 g, 1.54 mmol). The reaction was stirred at r.t. under dark for 6 h. The solution was diluted with 10 mL of water and purified directly by preparative HPLC to give 0.1 g (24%) of the desired compound as a yellow solid. Rf=0.28 (Heptane/EtOAc 75/25); ¹H NMR (600 MHz, CDCl₃) δ 6.11 (ddd, J=15.7, 10.8, 3.9 Hz, 1H), 5.13 (ddd, J=16.2, 9.5, 5.8 Hz, 1H), 4.53 (s, 2H), 3.77 (s, 3H), 3.35 (ddt, J=14.9, 11.1, 5.4 Hz, 2H), 3.26 (dd, J=11.2, 5.1 Hz, 1H), 3.08 (td, J=12.8, 2.8 Hz, 1H), 2.85-2.63 (m, 2H), 2.50-2.39 (m, 1H), 2.32 (ddd, J=11.1, 9.3, 5.5 Hz, 1H): ¹³C NMR (151 MHz, CDCl₃) δ 167.20, 164.20, 163.25, 143.86, 128.71, 52.87, 38.83, 35.36, 27.06.

### Example 3.3

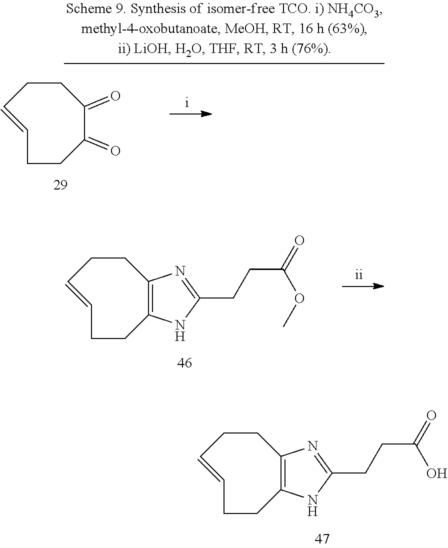 [SMILES: O=C1CC/C=C/CCC1=O ; COC(=O)CCc1nc2c([nH]1)CC/C=C/CC2 ; O=C(O)CCc1nc2c([nH]1)CC/C=C/CC2]

### Methyl (E)-3-(4,5,8,9-tetrahydro-1H-cycloocta[d]imidazol-2-yl)propanoate (46)

In a 15 mL screw neck vial ammonium bicarbonate (384 mg, 4.86 mmol) and methyl-4-oxobutanoate (85%, 331 mg, 2.43 mmol) was stirred in 2 mL MeOH at rt for 15 min. (E)-cyclooct-5-ene-1,2-dione (305 mg, 2.21 mmol) in 3 mL MeOH was added to the suspension and stirred for 16 h. The reaction mixture was diluted with 0.1% TFA(_{aq}.) and submitted to preparative HPLC to yield methyl (E)-3-(4,5,8,9-tetrahydro-1H-cycloocta[d]imidazol-2-yl)propanoate as a beige, viscous liquid (325 mg, 1.39 mmol, 63%). ¹H NMR (599.65 MHz, MeOD) δ=5.64-5.56 (m, 2H), 3.70 (s, 3H), 3.20-3.15 (m, 2H), 2.95 (dt, J=13.8, 6.5 Hz, 2H), 2.88 (t, J=7.0 Hz, 2H), 2.76-2.70 (m, 2H), 2.35 (d, J=6.1 Hz, 2H), 2.28 (h, J=6.7 Hz, 2H). ¹³C NMR (150.78 MHz, MeOD) δ=173.2, 136.0, 129.9, 56.6, 52.5, 31.5, 30.81, 29.0, 22.1.

### (E)-3-(4,5,8,9-Tetrahydro-1H-cycloocta[d]imidazol-2-yl)propanoic acid (47)

In a 5 mL screw neck vial 1M LiOH solution (861 μL, 861 μmol) was added to methyl (E)-3-(4,5,8,9-tetrahydro-1H-cycloocta[d]imidazol-2-yl)propanoate (100 mg, 287 μmol) in 2.5 mL THF/water (3:2 v/v). The suspension was stirred at rt for 3 h. The reaction was quenched with 1M TFA(_{aq}.) and submitted to preparative HPLC to yield (E)-3-(4,5,8,9-Tetrahydro-1H-cycloocta[d]imidazol-2-yl)propanoic acid as a light yellow, amorphous solid. (73 mg, 218 μmol, 76%). ¹H NMR (599.65 MHz, DMSO) δ=12.52 (s, 1H), 5.57-5.48 (m, 2H), 3.03 (t, J=7.2 Hz, 2H), 2.88 (dt, J=14.2, 6.4 Hz, 2H), 2.77 (t, J=7.2 Hz, 2H), 2.65 (ddd, J=14.4, 6.7, 4.9 Hz, 2H), 2.24 (ddd, J=11.5, 9.4, 4.5 Hz, 2H), 2.16 (ddd, J=12.1, 7.4, 4.3 Hz, 2H). ¹³C NMR (150.78 MHz, DMSO) δ=172.6, 142.67, 134.9, 127.6, 30.5, 29.2, 27.8, 20.8.

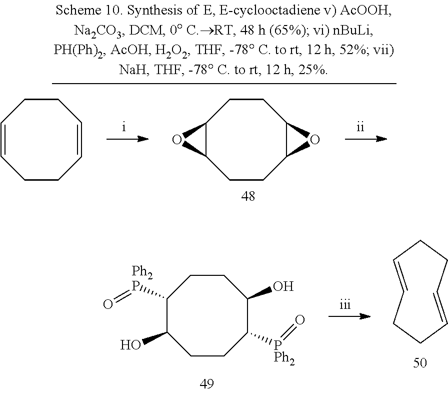

### cis,cis-5,10-Dioxatricyclo[7.1.0.04,6]decane (48)

The compound was synthesized as described in Stöckmann, H.; Neves, A. A.; Day, H. A.; Stairs, S.; Brindle, K. M.; Leeper, F. J. (E,E)-1,5-Cyclooctadiene: a small and fast click-chemistry multitalent. Chemical Communications 2011, 47, 7203-7205. Sodium carbonate (20.5 g, 50.84 mmol, 1.1 eq) was added to a stirred solution of (Z,Z)-1,5-cyclooctadiene (5 g, 46.2 mmol, 1.0 eq) in DCM (25 mL). The suspension was cooled to 0° C. and peracetic acid (36%, 17.9 mL, 97.1 mmol, 2.1 eq) was added. The mixture was warmed to r.t. and stirred until the reaction had gone to completion, after 48 hours. The reaction was quenched with sat. Na2CO3 solution, followed by extraction with EtOAc (4×25 mL). The combined organic layers were dried over MgSO4, concentrated and distilled to give cis,cis-5,10-dioxatricyclo[7.1.0.04,6]decane (4.19 g, 29.89 mmol, 65%). ¹H NMR (400 MHz, CDCl₃) δ 2.97-2.88 (m, 4H), 2.00-1.89 (m, 4H), 1.89-1.77 (m, 4H); ¹³C NMR (101 MHz, CDCl₃) δ 56.02, 22.01.

### ((Trans,trans)-2,6-dihydroxycyclooctane-1,5-diyl)bis(diphenylphosphine oxide) (49)

The compound was synthesized as described in Stöckmann, H.; Neves, A. A.; Day, H. A.; Stairs, S.; Brindle, K. M.; Leeper, F. J. (E,E)-1,5-Cyclooctadiene: a small and fast click-chemistry multitalent. Chemical Communications 2011, 47, 7203-7205. To a solution of diphenylphosphine (10.43 mL, 11.16 g, 59.92 mmol, 2.1 eq.) and cis,cis-5,10-dioxatricyclo-[7.1.0.04,6]decane (4.0 g, 28.5 mmol, 1.0 eq.) in THF (120 mL), BuLi (2 M solution in hexanes,29.96 mL, 59.92 mmol, 2.1 eq.) was added dropwise at −78° C. The mixture was stirred for 1 h, allowed to warm to room temperature and stirred for 12 h. The brown solution was cooled to 0° C., diluted with THF (86 mL) and quenched by the addition of AcOH (4.9 mL, 5.14 g, 85.60 mmol, 3 eq) and H₂O₂ (30%, 8.7 mL, 85.60 mmol, 3 eq). The mixture became clear, followed by the formation of a white precipitate. The mixture was removed from the ice bath and stirred vigorously at 25° C. for 2 h The suspension was then transferred to a separatory funnel, EtOAc (150 mL) and H₂O (50 mL) were added, the aqueous layer removed, and the organic layer washed with brine (2×20 mL). The precipitate was filtered off, which yield ((trans,trans)-5,8-dihydroxycyclooctane-1,4-diyl)bis(diphenylphosphine oxide). The combined organic layers were dried over MgSO4, filtered and concentrated to afford a white solid. The remaining AcOH was azeotropically removed with toluene to yield an isomeric mixture of phosphine oxides (8.14 g, 14.9 mmol, 52%). Spectroscopic data were in agreement with previously published data.

### (E,E)-1,5-cyclooctadiene (50)

The compound was synthesized as described in Stöckmann, H.; Neves, A. A.; Day, H. A.; Stairs, S.; Brindle, K. M.; Leeper, F. J. (E,E)-1,5-Cyclooctadiene: a small and fast click-chemistry multitalent. Chemical Communications 2011, 47, 7203-7205. The mixture of phosphine oxides (6.00 g, 11.0 mmol, 1.0 eq) was dissolved in DMF (30 mL) with stirring under an Ar atmosphere. The clear solution became cloudy and NaH (60% in mineral oil, 1.44 g, 34.2 mmol, 3.1 eq) was added in portions over 5 min at r.t. under a positive Ar stream. The reaction mixture was stirred for 12 h at room temperature, cooled to 0° C., diluted with pentane (50 mL) and quenched with half-saturated NH4Cl (22 mL). The aqueous layer was extracted with pentane (50 mL) and the combined organic layers were washed with water (5×22 mL) and brine (1×22 mL) to give (E,E)-1,5-cyclooctadiene (25% yield). Spectroscopic data were in agreement with previously published data.

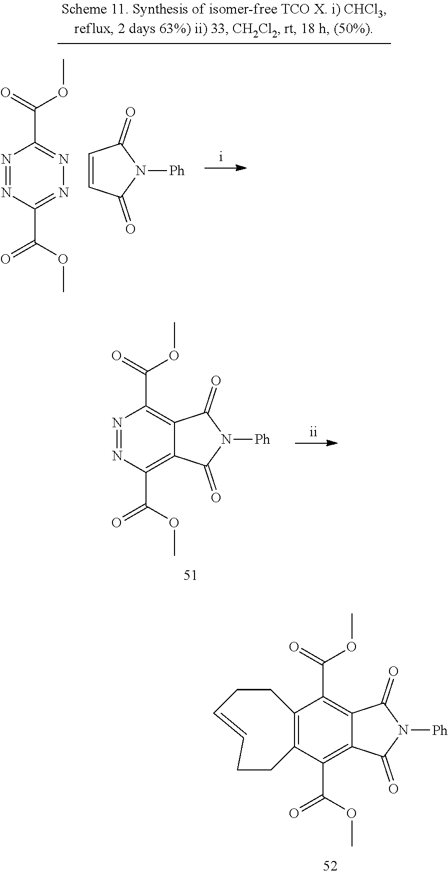

Dimethyl 6-benzyl-5,7-dioxo-6,7-dihydro-5H-pyrrolo[3,4-d]pyridazine-1,4-dicarboxylate (51) Dimethyl 1,2,4,5-tetrazine-3,6-dicarboxylate (50 mg, 0.25 mmol) was dissolved in CHCl₃ (1 mL), to which was added 1-phenyl-1H-pyrrole-2,5-dione (53 mg, 0.30 mmol) and the reaction was stirred for two days at reflux. Upon reaction, the dihydropyridazine spontaneously oxidized to the corresponding pyridazine. The reaction was cooled down to room temperature, upon complete consumption of dimethyl 1,2,4,5-tetrazine-3,6-dicarboxylate. The product was purified by CombiFlash, which yielded a pale solid (54 mg, 0.16 mmol, 63%). ¹H NMR (600 MHz, CDCl₃) δ 7.54 (dd, J=8.3, 6.8 Hz, 2H), 7.50-7.47 (m, 1H), 7.42-7.38 (m, 2H), 4.17 (s, 6H). ¹³C NMR (151 MHz, CDCl₃) δ 162.5, 162.2, 159.2, 149.5, 138.2, 130.3, 129.7, 126.5, 54.3.

### Dimethyl (E)-1,3-dioxo-2-phenyl-2,3,5,6,9,10-hexahydro-1H-cycloocta[f]isoindole-4,11-dicarboxylate (52)

To a solution of 35 (20 mg, 0.06 mmol) in CH₂Cl₂ was added compound 33 in pentane (0.06 mmol). The solution was stirred ra room temperature for 18 h. The volatiles were then removed under reduced pressure. Purification by flash chromatography afforded 12 mg (50%) of the desired product as a white solid. ¹H NMR (400 MHz, CDCl₃) δ 7.45 (dd, J=8.4, 6.9 Hz, 2H), 7.37 (t, J=7.4 Hz, 1H), 7.35-7.30 (m, 2H), 5.77 (t, J=5.5 Hz, 2H), 3.89 (s, 6H), 2.61-2.36 (m, 2H), 2.36-2.21 (m, 2H), 1.65 (d, J=8.1 Hz, 4H).

### Example 4

### Ligation Between Unsymmetrical Tetrazine and Isomer Free TCO

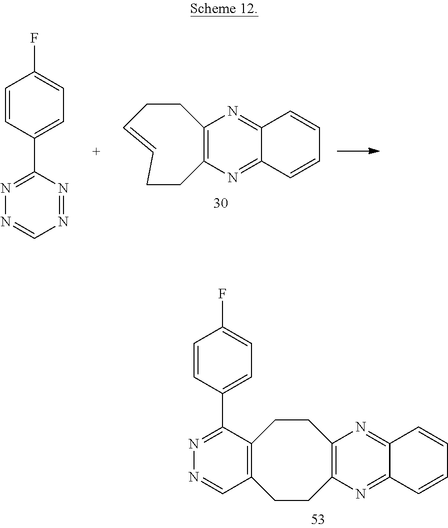 [SMILES: Fc1ccc(-c2nncnn2)cc1 ; C1=C/CCc2nc3ccccc3nc2CC/1 ; Fc1ccc(-c2nncc3c2CCc2nc4ccccc4nc2CC3)cc1]

### 1-(4-fluorophenyl)-5,6,13,14-tetrahydropyridazino[4′,5′:5,6]cycloocta[1,2-b]quinoxaline (53)

3-(4-Fluorophenyl)-1,2,4,5-tetrazine was obtained as described in Kjmr, A.; Petersen Ida, N.; Herth Matthias, M.; Kristensen Jesper, L. NOVEL TETRAZINE COMPOUNDS FOR IN VIVO IMAGING. WO 2020/108720 A1, 2019/11/29, 2020.

Compound 30 (12 mg, 0.057 mmol) was dissolved in mixture of MeCN/H₂O (4/1 v/v, 2.5 mL), to which was added 3-(4-fluorophenyl)-1,2,4,5-tetrazine (10 mg, 0.057 mmol). The reaction was stirred at RT for 30 minutes. p-Chloranil (17 mg, 0.068 mmol) was added and the reaction was stirred for 10 min. After which, the reaction was diluted with H₂O (5 mL) and filtered. The solution was concentrated under reduced pressure and purified by flash chromatography to give 10 mg (0.028 mmol) of the desired product. ¹H NMR (600 MHz, CDCl₃) δ 8.71 (s, 1H), 8.04-7.72 (m, 2H), 7.70-7.56 (m, 2H), 7.42 (ddd, J=8.5, 5.3, 1.0 Hz, 2H), 3.56 (t, J=7.4 Hz, 2H), 3.45 (t, J=7.4 Hz, 2H), 3.32 (t, J=7.4 Hz, 2H), 3.16 (t, J=7.5 Hz, 2H); ¹³C NMR (151 MHz, CDCl₃) δ 163.25 (d, J=248.9 Hz), 161.19, 154.70 (d, J=3.2 Hz), 151.67, 141.20, 141.01, 138.61, 136.66, 133.02 (d, J=3.5 Hz), 130.93 (d, J=8.3 Hz), 129.66, 128.54 (d, J=7.8 Hz), 122.46, 115.59 (d, J=21.6 Hz), 35.62, 34.52, 30.11, 27.39.

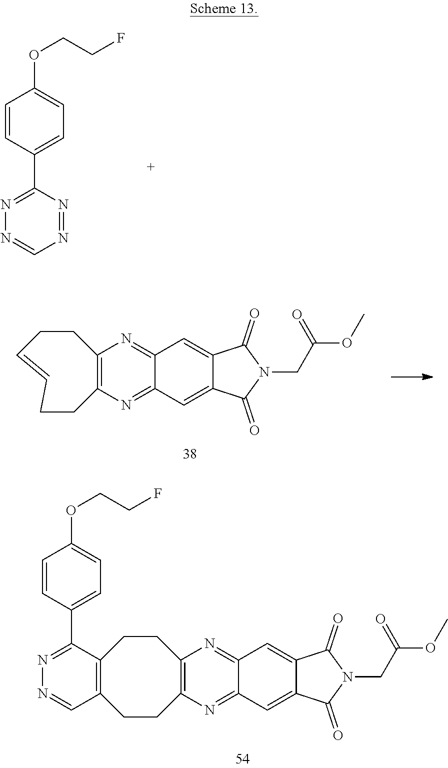 [SMILES: FCCOc1ccc(-c2nncnn2)cc1 ; COC(=O)CN1C(=O)c2cc3nc4c(nc3cc2C1=O)CC/C=C/CC4 ; COC(=O)CN1C(=O)c2cc3nc4c(nc3cc2C1=O)CCc1c(cnnc1-c1ccc(OCCF)cc1)CC4]

### 2-(1-(4-(2-Fluoroethoxy)phenyl)-9,11-dioxo-5,6,9,11,14,15-hexahydro-10H-pyridazino[4′,5′:5,6]cycloocta[1,2-b]pyrrolo[3,4-g]quinoxalin-10-yl)acetic acid (54)

3-(4-(2-Fluoroethoxy)phenyl)-1,2,4,5-tetrazine was obtained as described in Garcia Vázquez et al. Pharmaceuticals 2022, 15(2), 245. 3-(4-(2-Fluoroethoxy)phenyl)-1,2,4,5-tetrazine (3.2 mg, 0.0148 mmol) was dissolved in MeCN (1 mL) and added to a stirred solution of methyl (E)-2-(1,3-dioxo-1,3,6,7,10,11-hexahydro-2H-cycloocta[b]pyrrolo[3,4-g]quinoxalin-2-yl)acetic acid (5 mg, 0.0148 mmol) in H₂O (2 mL). p-Chloranil (18 mg, 0.074 mmol) was added and the reaction was stirred for 15 min. After which, the reaction was diluted with H₂O (15 mL) and filtered. The solution was concentrated under reduced pressure and purified by preparative HPLC and all fractions containing the pure product were lyophilized to yield the product as a white solid (5 mg, 0.0095 mmol, 64%). ¹H NMR (600 MHz, DMSO-d₆) δ 13.31 (br s, 1H), 8.92 (d, J=1.9 Hz, 1H), 8.42 (d, J=8.5 Hz, 2H), 7.43-7.35 (m, 2H), 7.22-7.14 (m, 2H), 4.87-4.84 (m, 1H), 4.80-4.76 (m, 1H), 4.41-4.36 (m, 3H), 4.36-4.33 (m, 1H), 3.68 (t, J=7.2 Hz, 2H), 3.57 (t, J=7.2 Hz, 2H), 3.44 (t, J=7.3 Hz, 2H), 3.27 (d, J=14.6 Hz, 2H); ¹³C NMR (151 MHz, DMSO) δ 169.03, 166.40, 166.38, 161.23, 159.76, 159.61, 159.07, 151.52, 143.51, 143.39, 139.90, 137.24, 130.95, 130.93, 130.84, 129.80, 125.13, 125.08, 114.91, 82.64 (d, J=166.5 Hz), 67.68 (d, J=18.9 Hz), 35.03, 34.82, 28.80, 27.08.

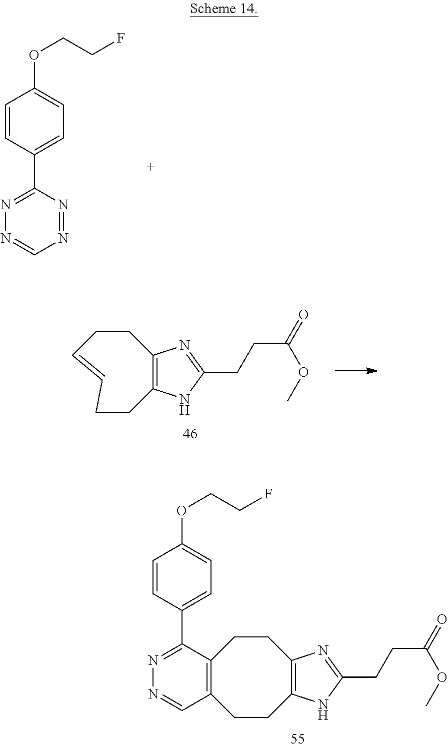 [SMILES: FCCOc1ccc(-c2nncnn2)cc1 ; COC(=O)CCc1nc2c([nH]1)CC/C=C/CC2 ; COC(=O)CCc1nc2c([nH]1)CCc1cnnc(-c3ccc(OCCF)cc3)c1CC2]

### Methyl 3-(6-(4-(2-fluoroethoxy)phenyl)-4,5,10,11-tetrahydro-1H-imidazo[4′,5′:5,6]cycloocta[1,2-d]pyridazin-2-yl)propanoate 2,2,2-trifluoroacetate (55)

3-(4-(2-fluoroethoxy)phenyl)-1,2,4,5-tetrazine was obtained as described in Garcia Vázquez et al. Pharmaceuticals 2022, 15(2), 245. 3-(4-(2-Fluoroethoxy)phenyl)-1,2,4,5-tetrazine (9 mg, 0.043 mmol) was dissolved in MeCN (1 mL) and added to a stirred solution of methyl (E)-3-(4,5,8,9-tetrahydro-1H-cycloocta[d]imidazol-2-yl)propanoate (15 mg, 0.043 mmol) in MeCN/H₂O (50 v/v %, 1 mL). p-Chloranil (53 mg, 0.215 mmol) was added and the reaction was stirred for 25 min. After which, the reaction was diluted with H₂O (25 mL) and filtered. The solution was concentrated under reduced pressure and purified by preparative HPLC and all fractions containing the pure product were lyophilized, which yield the product as yellow solid (5 mg, 0.012 mmol, 27%). ¹H NMR (600 MHz, MeOD) δ 8.99 (s, 1H), 7.41 (d, J=8.7 Hz, 2H), 7.15 (d, J=8.6 Hz, 2H), 4.83-4.72 (m, 2H), 4.37-4.28 (m, 2H), 3.62 (s, 3H), 3.37 (t, J=7.0 Hz, 2H), 3.26 (t, J=7.0 Hz, 2H), 3.21 (t, J=7.0 Hz, 2H), 3.05 (t, J=6.9 Hz, 2H), 2.98 (t, J=7.0 Hz, 2H), 2.79 (t, J=6.9 Hz, 2H).

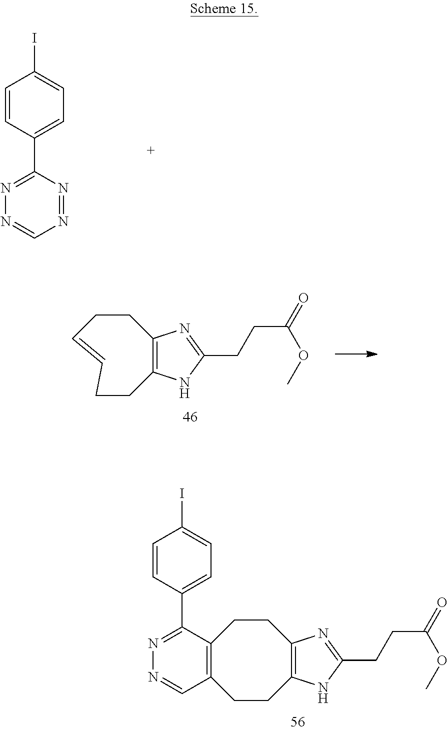 [SMILES: Ic1ccc(-c2nncnn2)cc1 ; COC(=O)CCc1nc2c([nH]1)CC/C=C/CC2 ; COC(=O)CCc1nc2c([nH]1)CCc1cnnc(-c3ccc(I)cc3)c1CC2]

### Methyl 3-(6-(4-iodophenyl)-4,5,10,11-tetrahydro-1H-imidazo[4′,5′:5,6]cycloocta[1,2-d]pyridazin-2-yl)propanoate 2,2,2-trifluoroacetate (56)

3-(4-lodophenyl)-1,2,4,5-tetrazine was obtained as described in Garcia Vazquez et al. Pharmaceuticals 2022, 15(2), 245. 3-(4-lodophenyl)-1,2,4,5-tetrazine (16 mg, 0.057 mmol) was dissolved in MeCN (1 mL) and added to a stirred solution of methyl (E)-3-(4,5,8,9-tetrahydro-1H-cycloocta[d]imidazol-2-yl)propanoate (20 mg, 0.057 mmol) in MeCN/H₂O (50 v/v %, 1 mL). p-Chloranil (71 mg, 0.287 mmol) was added and the reaction was stirred for 15 min. After which, the reaction was diluted with H₂O (20 mL) and filtered. The solution was concentrated under reduced pressure and purified by preparative HPLC and all fractions containing the pure product were lyophilized, which yield the product as yellow solid (21 mg, 0.034 mmol, 61%). ¹H NMR (600 MHz, MeOD) δ 9.03 (s, 1H), 7.94 (d, J=8.3 Hz, 2H), 7.24 (d, J=8.3 Hz, 2H), 3.62 (s, 3H), 3.37 (t, J=7.0 Hz, 2H), 3.21 (td, J=7.0, 4.0 Hz, 4H), 3.05 (t, J=6.9 Hz, 2H), 2.97 (t, J=7.0 Hz, 2H), 2.79 (t, J=7.0 Hz, 2H). ¹³C NMR (151 MHz, MeOD) δ 173.11, 162.90, 153.09, 146.19, 142.67, 139.88, 139.08, 137.32, 131.95, 128.12, 127.91, 96.19, 52.53, 31.21, 28.48, 26.91, 26.11, 24.82, 22.02.

### Example 5

### Ligation Between Symmetric Tetrazine and Isomer-Free TCO

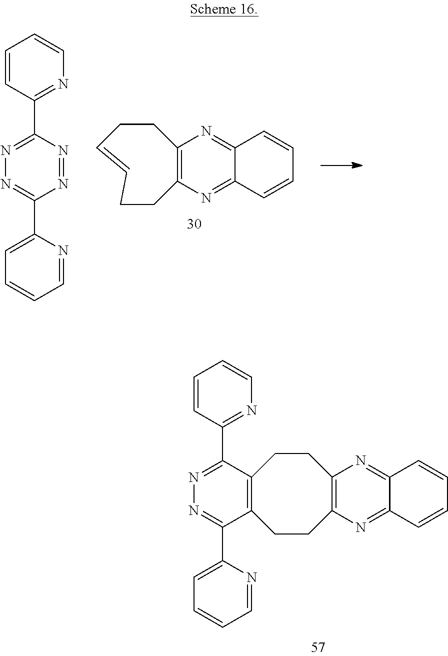

### 1,4-di(pyridin-2-yl)-5,6,13,14-tetrahydropyridazino[4′,5′:5,6]cycloocta[1,2-b]quinoxaline. (57)

(50 mg, 0.24 mmol) was dissolved in mixture of MeCN/H₂O (4/1 v/v, 5 mL), to which was added 3,6-di(pyridin-2-yl)-1,2,4,5-tetrazine (56 mg, 0.24 mmol). The reaction was stirred for 30 minutes at rt. p-Chloranil (117 mg, 0.47 mmol) was added, and the reaction was stirred for 10 min. After which, the reaction was diluted with H₂O (50 mL) and filtered. The solution was concentrated under reduced pressure and purified by flash chromatography to give 80 mg (0.19 mmol) of the desired product. ¹H NMR (400 MHz, CDC₃) δ 8.79 (d, J=4.8 Hz, 2H), 8.05-7.93 (m, 4H), 7.91 (t, J=7.7 Hz, 2H), 7.68 (dt, J=6.4, 3.7 Hz, 2H), 7.42 (dd, J=7.5, 4.9 Hz, 2H), 3.62 (t, J 5=6.4 Hz, 4H), 3.51 (t, J=6.6 Hz, 4H); ¹³C NMR (101 MHz, CDC₃) δ 158.39, 156.56, 156.12, 148.64, 140.73, 140.42, 137.11, 129.42, 128.43, 125.09, 123.61, 36.57, 27.69.

### Radiolabelling

### General Methods

All reagents and solvents were purchased from ABX, Sigma Aldrich, Fluorochem and VWR and used as received, without further purification, unless stated otherwise. Dry THF and DCM were obtained from a SG Water solvent purification system and dry dimethyl sulfoxide (DMSO), MeCN, pyridine and methanol (MeOH) were purchased from commercial suppliers. Room temperature corresponds to a temperature interval from 18-21° C. Reactions requiring anhydrous conditions were carried out under inert atmosphere (nitrogen) and using oven-dried glassware (152° C.). Thin-layer chromatography (TLC) was run on silica plated aluminum sheets (Silica gel 60 F254) from Merck and the spots were visualized by ultraviolet light at 254 nm, by anisaldehyde and/or by potassium permanganate staining.

### Example 6

### ¹⁸F Radiolabeling of Symmetrical Tetrazines

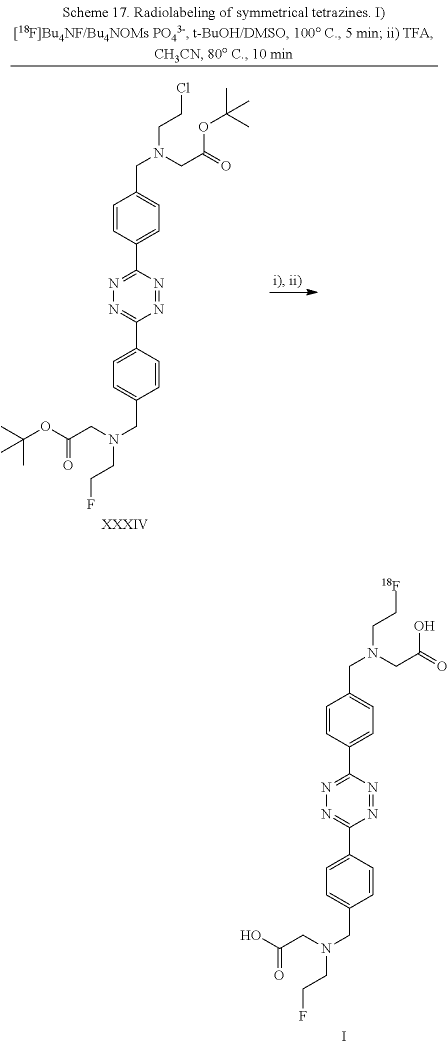

Symmetrical ¹⁸F-labeled tetrazine [¹³F]I was prepared as follows:

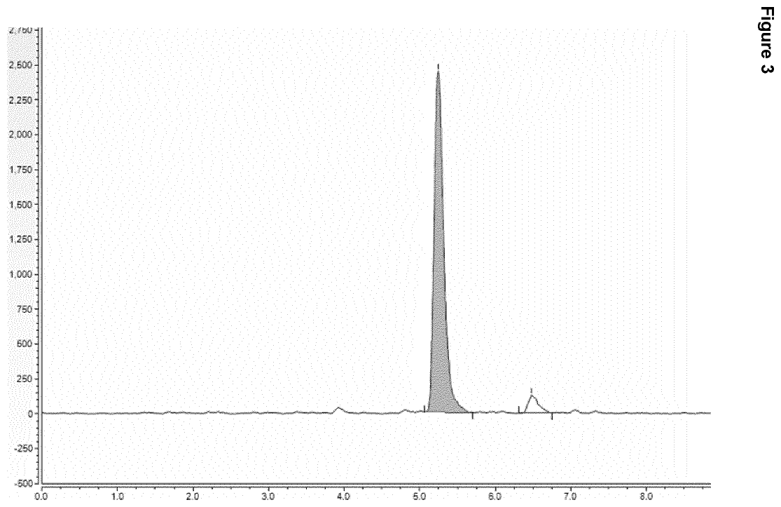

The aqueous [¹⁸F]fluoride solution received from the cyclotron was passed through a preconditioned anion exchange resin (Sep-Pak Light QMA cartridge). The QMA was preconditioned by flushing it with 10 mL 0.5 M K₃PO₄ and washing it with 10 mL H₂O afterwards. [¹⁸F]F—was eluted from the QMA into a 4 mL v-shaped vial with 1 mL Bu₄NOMs dissolved in MeOH. The eluate was dried at 100° C. for 5 min under N₂-flow. Precursor XXXIV (9.3 μmol, 6 mg) was dissolved in 167 μL DMSO and then diluted with 833 μL tBuOH. The solution was added to the dried [¹⁸F]fluoride solution and allowed to react for 5 min at 100° C. The reaction was cooled to 50° C. with air before addition of 3 mL H₂O. Radiochemical conversion (RCC) determined by radio-HPLC after the first step was 54%. The crude mixture was applied to a Sep-pak plus C18 solid phase extraction (SPE) cartridge that was preconditioned by flushing it with 10 mL EtOH followed by 10 mL of H₂O. The SPE was flushed with another 5 mL of H₂O and dried with N₂. The product was eluted from the SPE with 2 mL MeCN into a 7 mL v-shaped vial containing 600 μL TFA. This mixture was reacted for 10 min at 80° C. The RCC of [¹⁸F]I determined by radio-HPLC was 95% (FIG. 3).

Radio-HPLC was performed on a Luna 5 μm C18(2) column (150×4.6 mm) using a gradient of acetonitrile (CH₃CN) in water with 0.1% TFA. Gradient conditions: 0 min—0% CH₃CN, 0-10 min—linear increase of CH₃CN content to 100%, 10-12 min—100% CH₃CN, 12-13 min—linear decrease of CH₃CN content to 0%, 13-15 min—0% CH₃CN, elution speed 2 mL/min.

FIG. 3 shows Radio-HPLC of [¹⁸F]I at end of deprotection.

### Example 7

### ¹⁸F Radiolabeling of Unsymmetrical Tetrazines

Unsymmetrical ¹⁸F-labeled tetrazine [18F]XIII was prepared from the nosyl precursor XXXI via nucleophilic substitution as disclosed in Battisti, U. M.; Bratteby, K.; JØrgensen, J. T.; Hvass, L.; Shalgunov, V.; Mikula, H.; Kjwr, A.; Herth, M. M. Development of the First Aliphatic 18F-Labeled Tetrazine Suitable for Pretargeted PET Imaging—Expanding the Bioorthogonal Tool Box. J. Med. Chem. 2021, 64, 15297-15312

### Example 8

### ¹⁸F-Radiolabeling of Unsymmetrical Tetrazines

¹⁸F-Labeled tetrazines were prepared using a general method described in Garcia-Vázquez et al. (doi: 10.3390/ph15020245) with minor modifications.

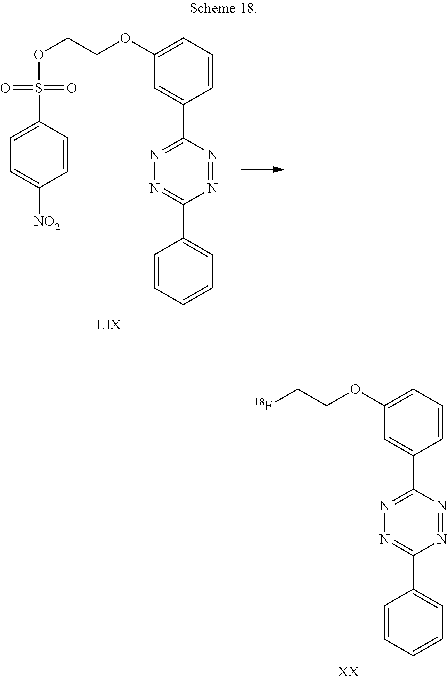

The aqueous [¹⁸F]fluoride solution received from the cyclotron was passed through a Sep-Pak Light QMA cartridge preconditioned with 10 mL 0.5 M K₃PO₄. [¹⁸F]F—was eluted from the QMA into a 4 mL v-shaped vial using Bu₄NOMs solution (20 mM in MeOH, ¹ mL). The eluate was dried at 100° C. for 5 min under nitrogen or helium flow. After MeOH had evaporated, acetonitrile (0.5 mL) was added to the same vial and evaporated under the same conditions to remove traces of water. Two additions of acetonitrile were performed. Nosyl precursor LIX (1.5 mg) was dissolved in anhydrous DMSO (0.2 mL), diluted with tBuOH (0.8 mL) and added to the dried [¹⁸F]fluoride residue. After reacting for 5 min at 100° C., the reaction was cooled to 80° C. with ambient air flow, diluted with water (2 mL) and purified by semipreparative HPLC: Luna 5 μm C18(2) 100 Å, 250 mm×10 mm column, isocratic elution with 66% acetonitrile in 20 mM citrate buffer pH 6.1, elution speed 6 mL/min. The product peak (retention time 10.2 min) was collected, diluted with water (50-100 mL) and passed through a Sep-pak Plus C18 Short solid phase extraction cartridge (Waters, USA) that was preconditioned by flushing it with EtOH/water mixture (1/1 v/v, 10 mL). The Sep-pak cartridge was then flushed with extra water (5 mL), blown with nitrogen, eluted with 1-2 mL of organic solvent (MeCN or EtOH) and diluted with water to achieve the necessary concentration of the organic solvent for click/oxidation experiments. [¹³F]XX was obtained in a radiochemical yield of 39±13% (n=4), >95% radiochemical purity.

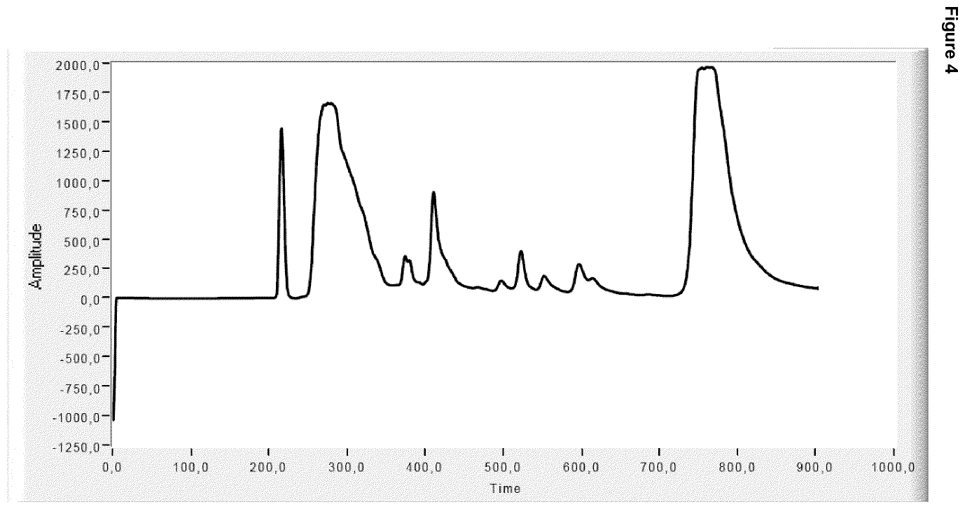

FIG. 4 shows the UV trace of [¹³F]XX

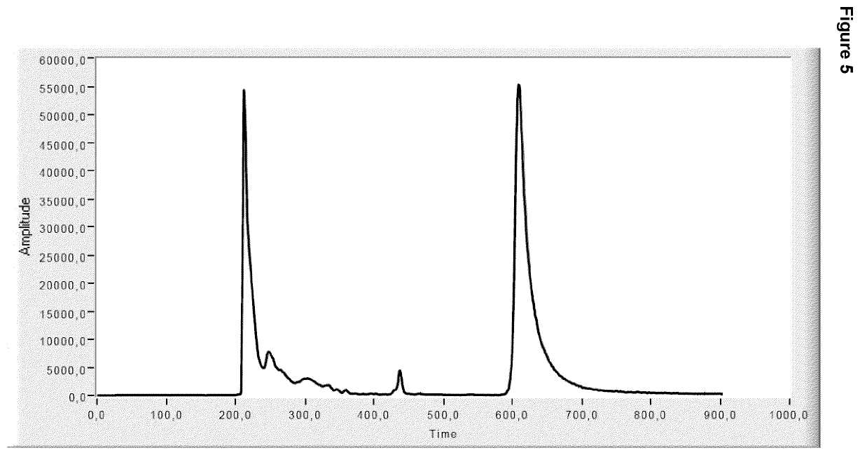

FIG. 5 shows the radioactivity trace of [¹³F]XX

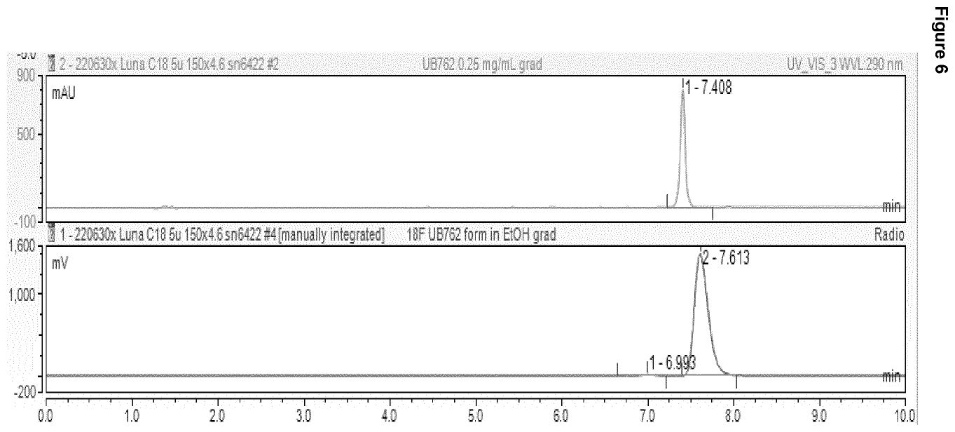

FIG. 6 shows UV and radioactivity trace of [¹⁸F]XX—analytical HPLC

HPLC conditions: Luna 5 μm C18(2) column (150×4.6 mm) eluted with a gradient of acetonitrile (CH₃CN) in water with 0.1% TFA. Gradient conditions: 0-1 min—25% CH₃CN, 1-8 min—linear increase of CH₃CN content to 95%, 8-9 min—95% CH₃CN, 9-9.5 min—linear decrease of CH₃CN content to 25%, 9.5-10 min—25% CH₃CN, elution speed 1.5 mL/min.

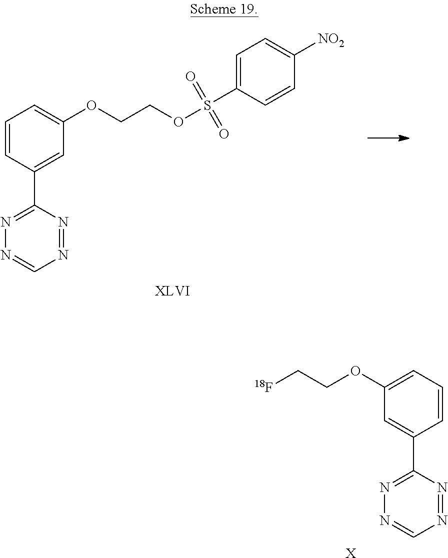

The aqueous [¹⁸F]fluoride solution received from the cyclotron was passed through Sep-Pak Light QMA cartridge preconditioned with 10 mL 0.5 M K₃PO₄. [¹⁸F]F—was eluted from the QMA cartridge into a 4 mL v-shaped vial using Bu₄NOTf solution (20 mM in MeOH, ¹ mL). The eluate was dried at 100° C. for 5 min under nitrogen or helium flow. After MeOH had evaporated, acetonitrile (0.5 mL) was added to the same vial and evaporated under the same conditions to remove traces of water. Nosyl precursor XLVI (1.5 mg) was dissolved in anhydrous acetonitrile (0.2 mL), diluted with tBuOH (0.8 mL) and added to the dried [¹⁸F]fluoride residue. After reacting for 5 min at 100° C., the reaction was cooled to 80° C. with ambient air flow, diluted with water (2 mL) and purified by semipreparative HPLC: Luna 5 μm C18(2) 100 Å, 250 mm×10 mm column, isocratic elution with 55% acetonitrile in 20 mM citrate buffer pH 6.1, elution speed 5 mL/min. The product peak (retention time 11.5 min) was collected, diluted with water (50-100 mL) and passed through a Sep-pak Plus C18 Short solid phase extraction cartridge (Waters, USA) that was preconditioned by flushing it with EtOH/water mixture (1/1 v/v, 10 mL). The Sep-pak cartridge was then flushed with extra water (5 mL), blown with nitrogen, eluted with 1-2 mL of organic solvent (MeCN or EtOH) and diluted with water to achieve the necessary concentration of the organic solvent for click/oxidation experiments.

[¹⁸F]X was obtained in a radiochemical yield of 11±5% (n=3), >95% radiochemical purity.

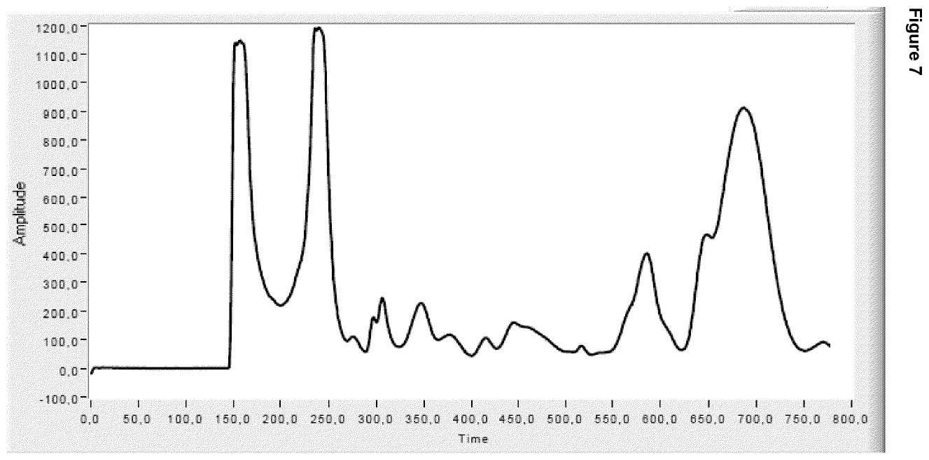

FIG. 7 shows the UV trace of [¹⁸F]X—Semi-prep HPLC

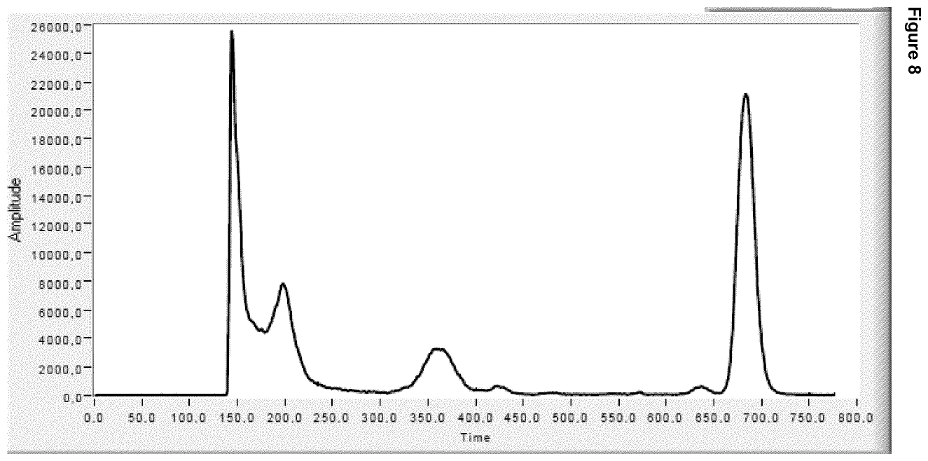

FIG. 8 shows radioactivity trace of [¹⁸F]X—Semi-prep HPLC

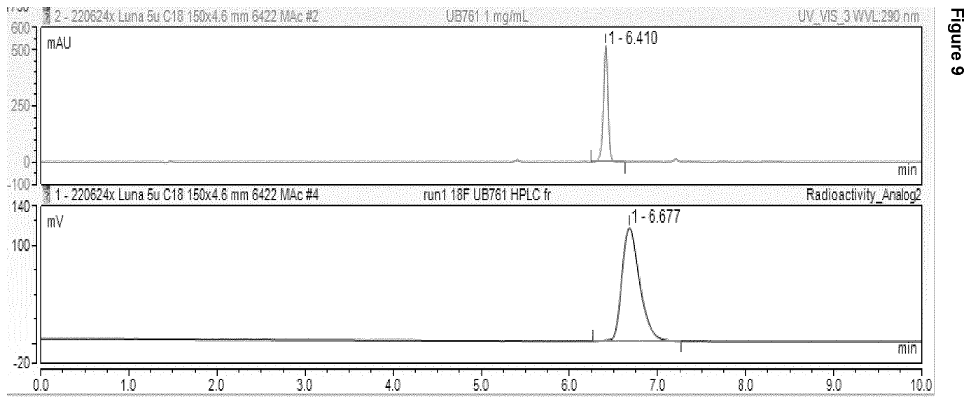

FIG. 9 shows UV and radioactivity trace of [¹⁸F]X—analytical HPLC

HPLC conditions: Luna 5 μm C18(2) column (150×4.6 mm) eluted with a gradient of acetonitrile (CH₃CN) in water with 0.1% TFA. Gradient conditions: 0-1 min—15% CH₃CN, 1-8 min—linear increase of CH₃CN content to 85%, 8-9 min—85% CH₃CN, 9-9.5 min—linear decrease of CH₃CN content to 15%, 9.5-10 min—15% CH₃CN, elution speed 1.5 mL/min.

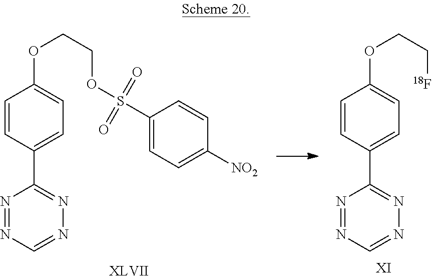

The aqueous [¹⁸F]fluoride solution received from the cyclotron was passed through a 45 mg PS anion exchange cartridge (Synthra) preconditioned with 5 mL 0.5 M K₃PO₄. [¹⁸F]F—was eluted from the PS cartridge into a 4 mL v-shaped vial using Bu₄NOTf solution (20 mM in MeOH, ^{0.6} mL). The eluate was dried at 100° C. for 5 min under nitrogen or helium flow. After MeOH had evaporated, acetonitrile (0.5 mL) was added to the same vial and evaporated under the same conditions to remove traces of water. Two additions of acetonitrile were performed. Nosyl precursor XLVII (1.5 mg) was dissolved in anhydrous acetonitrile (0.3 mL), diluted with tBuOH (0.7 mL) and added to the dried [¹⁸F]fluoride residue. After reacting for 5 min at 100° C., the reaction was cooled to 80° C. with ambient air flow, diluted with water (2 mL) and purified by semipreparative HPLC: Discovery HS F5 5 μm, 250 mm×10 mm column, isocratic elution with 50% acetonitrile in 20 mM citrate buffer pH 6.1, elution speed 5 mL/min. The product peak (retention time 9.0 min) was collected, diluted with water (50-100 mL) and passed through a Sep-pak Plus C18 Short solid phase extraction cartridge (Waters, USA) that was preconditioned by flushing it with EtOH/water mixture (1/1 v/v, 10 mL). The Sep-pak cartridge was then flushed with extra water (5 mL), blown with nitrogen, eluted with 1-2 mL of organic solvent (MeCN or EtOH) and diluted with water to achieve the necessary concentration of the organic solvent for click/oxidation experiments.

[¹⁸F]XI was obtained in a radiochemical yield of 16±10% (n=4), >95% radiochemical purity.

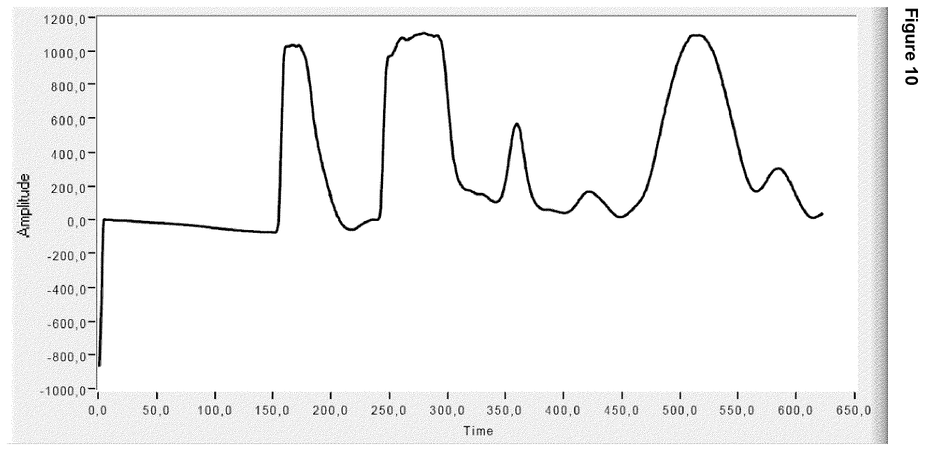

FIG. 10 shows UV trace—Semi-prep HPLC of [¹⁸F]XI

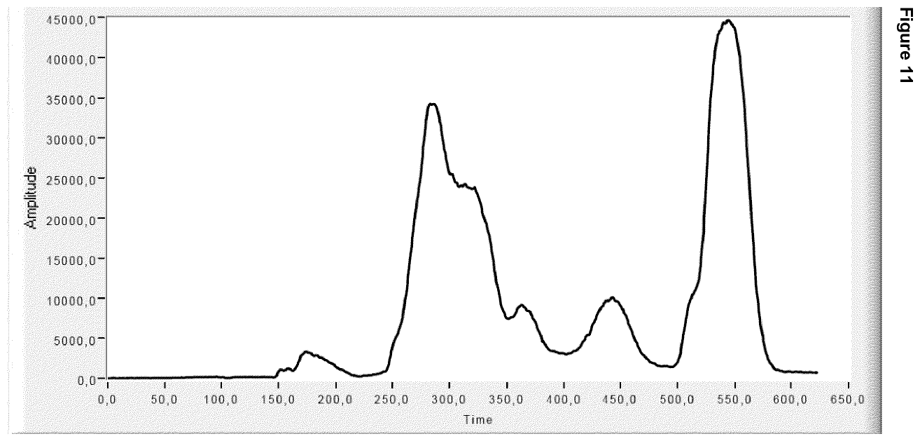

FIG. 11 shows radioactivity trace—Semi-prep HPLC of [¹⁸F]XI

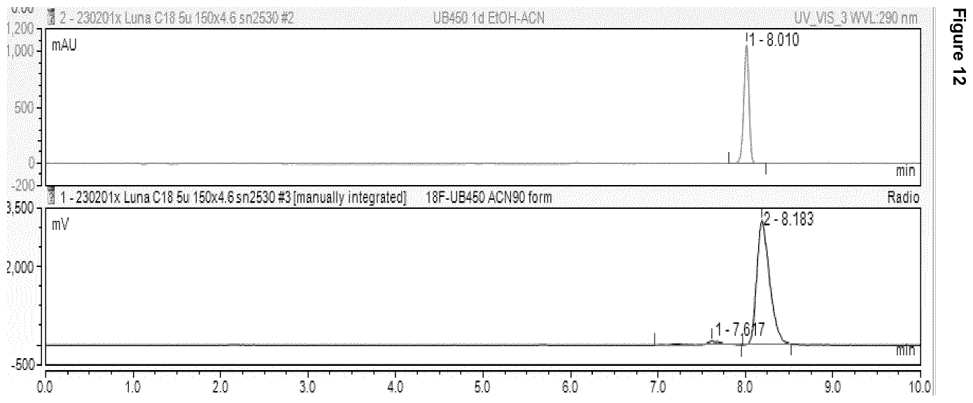

FIG. 12 shows UV and radioactivity trace of [¹⁸F]XI—analytical HPLC

HPLC conditions: Luna 5 μm C18(2) column (150×4.6 mm) eluted with a gradient of acetonitrile (CH₃CN) in water with 0.1% TFA. Gradient conditions: 0-1 min—25% CH₃CN, 1-8 min—linear increase of CH₃CN content to 60%, 8-9 min—60% CH₃CN, 9-9.5 min—linear decrease of CH₃CN content to 25%, 9.5-10 min—25% CH₃CN, elution speed 1.5 mL/min.

### Example 9

### ¹²⁵I-Radiolabeling of Unsymmetrical Tetrazines

Unsymmetrical ¹²⁵I-labeled tetrazine [¹²⁵I]XVII was prepared from the stannyl precursor LVI as disclosed in Battisti Umberto, M.; Herth Matthias, M.; Kjmr, A.; Garcia, R. NUCLIDE LABELLED H-TETRAZINES AND USE THEREOF FOR PET AND SPECT PRETARGETED IMAGING AND RADIONUCLIDE THERAPY. WO 2021/228992 A1, 2021/05/12, 2021

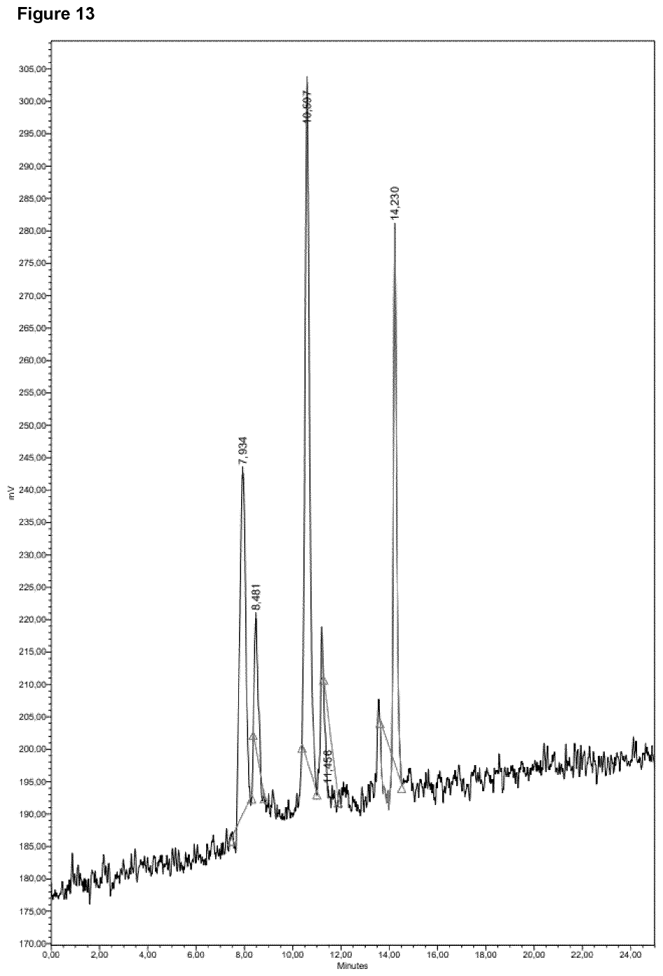

FIG. 13 shows radio-HPLC of crude [¹²⁵I]XVII.

### Example 10

### ²¹¹At-Radiolabeling of Unsymmetrical Tetrazines

Unsymmetrical ²¹¹At-labeled tetrazine [²¹¹At]XIV was prepared from the stannyl precursor LIV as disclosed in Battisti Umberto, M.; Herth Matthias, M.; Kjmr, A.; Garcia, R. NUCLIDE LABELLED H-TETRAZINES AND USE THEREOF FOR PET AND SPECT PRETARGETED IMAGING AND RADIONUCLIDE THERAPY. WO 2021/228992 A1, 2021/05/12, 2021

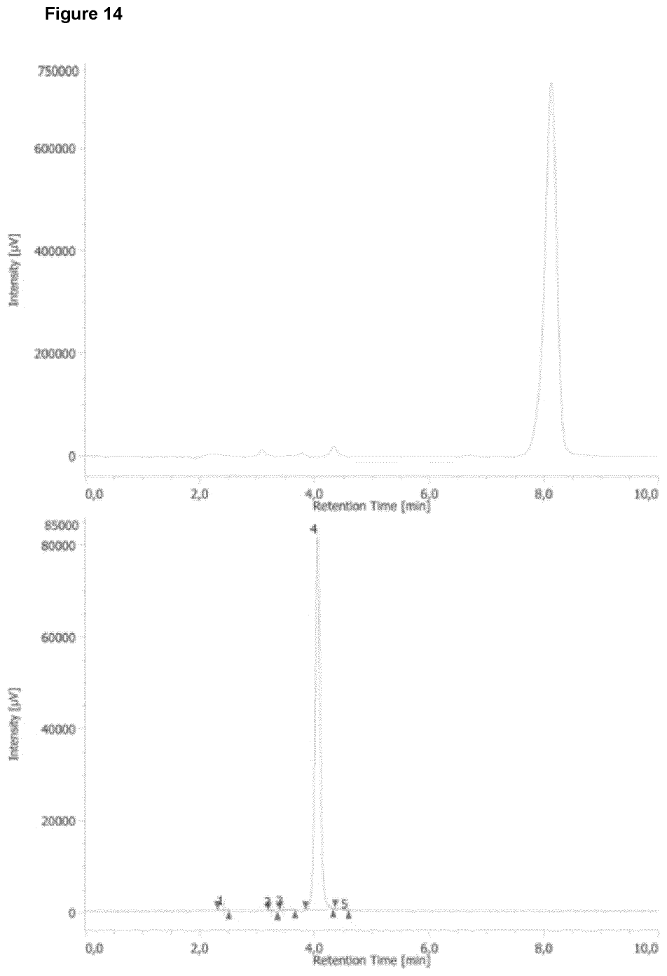

FIG. 14 shows UV and radioactivity trace—Semi-prep HPLC of [²¹¹At]XIV

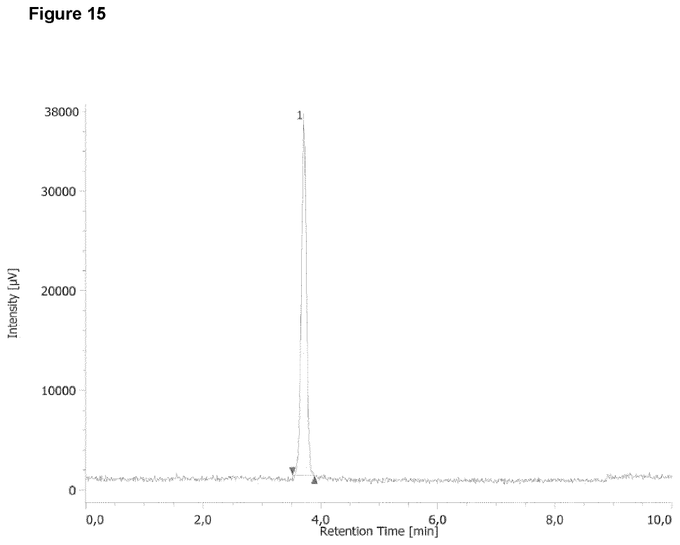

FIG. 15 shows radioactivity trace of purified [²¹¹At]XIV—analytical HPLC

### Example 11

Click Experiments with Radiolabeled Tetrazines

In click experiments, an excess of TCO was reacted with non-carrier added radiolabeled ¹⁸F-Tz. The half-life of a bimolecular reaction is determined by the concentration of the reagent which is in excess. Therefore, in click experiments, the observed conversion times were determined by TCO concentration, and all 18F-Tz was consumed.

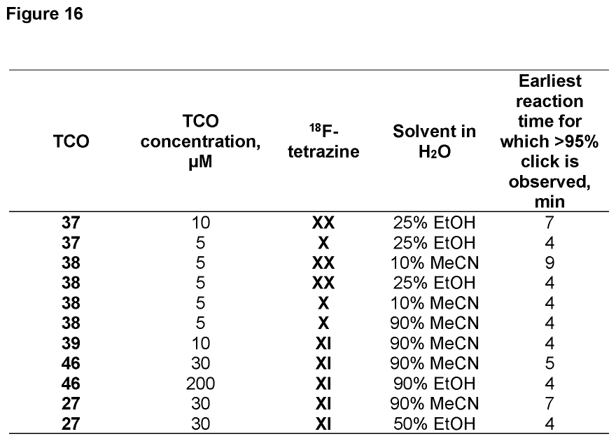

TCO stock solution (20-2000 μM) was prepared in solvent mixture matching that of the formulated ¹⁸F-Tz solution. Then, TCO stock was quickly mixed with formulated 18F-Tz solution at vol/vol ratios of 1:3 to 1:9 to achieve the desired TCO concentration (5-200 μM). The total volume of the mixture was 100-2000 μL. The mixture was left standing at room temperature for a certain time, and then a sample was injected on radio-HPLC to assess click conversion. The results are shown in FIG. 16.

### Example 12

### Screening of Oxidants for the Oxidation of the Dihydropyridazines to Pyridazines, Yielding Single End-Products

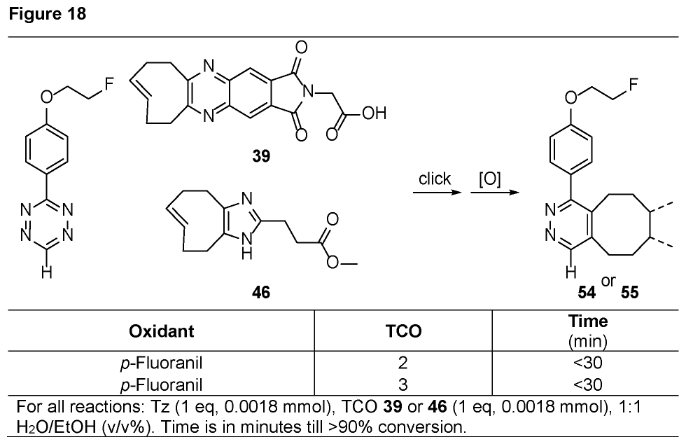

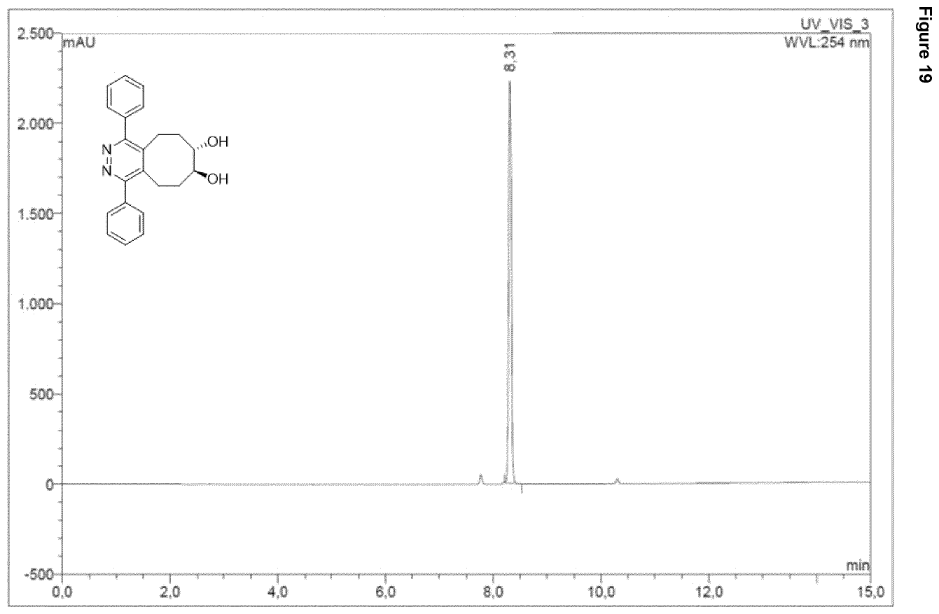

The top of FIG. 17 and of FIG. 18 shows the reactions between a tetrazine and a TCO, dissolved in 1:1 H₂O/EtOH (% v/v). The cycloaddition is completed within 5 minutes to give several isomers. The oxidants is then added to give the final single isomeric product. Each oxidant (5 equivalents) was added to the mixture and the reaction was analyzed by HPLC-MS after 60 minutes. These screening tests surprisingly showed that not all oxidants could be applied to provide a single isomeric form of the tetrazine-TCO pyridazine. For all the quinone-based oxidants, the corresponding pyridzine is formed. The inorganic-based examples are not able to transform the click-product (dihydropyridazines) to the corresponding pyridzine, within 60 minutes. FIG. 19 shows the HPLC analysis after oxidation of the tetrazine-TCO pyridazine tested.

### Oxidations of Radiolabeled Compounds

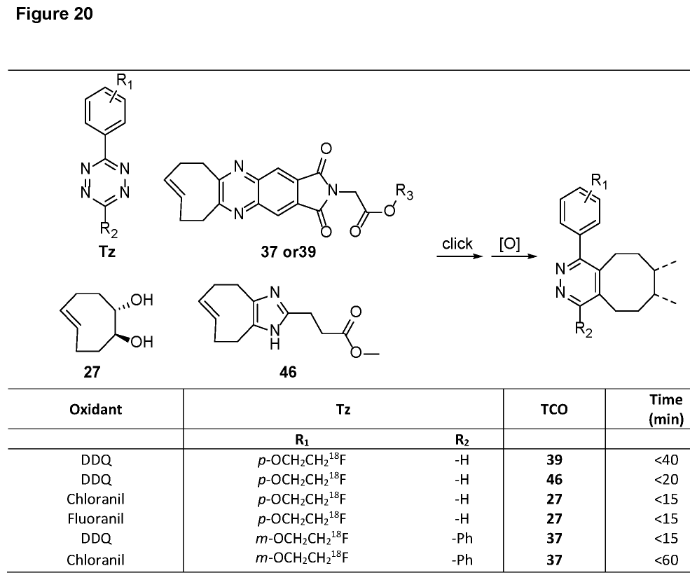

The top of FIG. 20 shows the reactions between a ¹⁸F-Tetrazine and a TCO, dissolved in 1:1 H₂O/EtOH (% v/v). The crude solution of ¹⁸F-click product (50-100 μL), obtained by mixing ¹⁸F-Tz and TCO solutions as described in the Click section, was used for oxidation experiments. Oxidation protocol consisted of introduction of oxidant. The oxidation mixture was left standing in a closed vial at room temperature for the desired time period, then a sample was withdrawn and analyzed by HPLC.

### Example 13

Compatibility of Targeting Vectors with Oxidation Conditions:

In order to test whether the conditions leading to the oxidation of click product will not lead to the degradation of typical targeting vectors, we subjected a series of vectors relevant for theranostic radiopharmaceutical development to oxidation conditions previously shown to result in efficient conversion of dihydropyridazines to single-product pyridazines.

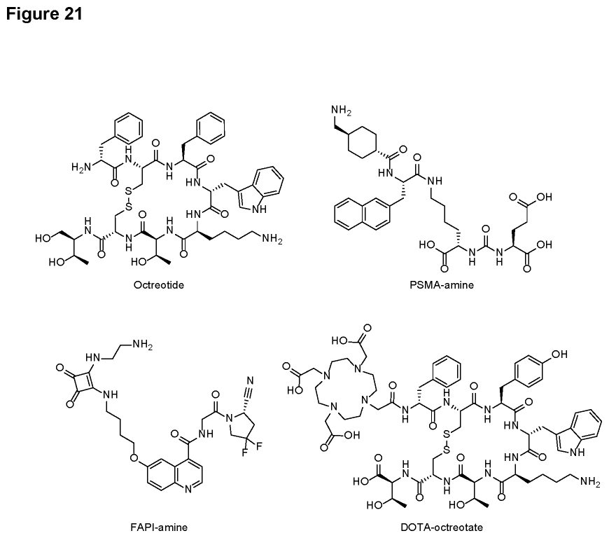

Structures of tested vectors are shown in FIG. 21.

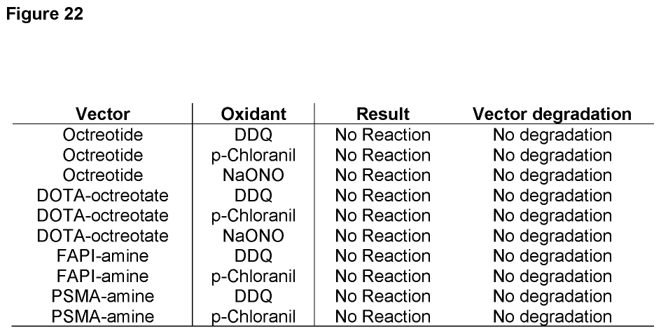

Vector oxidation test procedure: solution of targeting vector (70 μM) and oxidant (350 μM, 5 eq) in EtOH/water mixture (89-94% EtOH v/v) was stirred for 10 min at 25° C. and subsequently analysed by analytical HPLC and LC/ESI-MS. None of the tested compounds showed oxidation and/or degradation meaning that they do not react with oxidants. The results are shown in FIG. 22 and showed that these vectors are compatible with the tested oxidants.

### Example 14

### Measurement of Second-Order Rate Constants

The second-order rate constant of all the click reactions made during the previous examples were measured by stopped-flow spectrometry in phosphate-buffered saline (PBS) at 25° C. in accordance with the method described in Battisti et al. J. Med. Chem. 2021, 64, 20, 15297-15312 (see page 15310 for experimental details and influencing factors). In short, stopped-flow measurements were performed using an SX20-LED stopped-flow spectrophotometer (Applied Photophysics) equipped with a 535 nm LED (optical pathlength 10 mm and full width half-maximum 34 nm) to monitor the characteristic tetrazine visible light absorbance (520-540 nm). The reagent syringes were loaded with a solution of axial-TCO-PEG₄, and the instrument was primed. The subsequent data were collected in triplicate for each tetrazine. Reactions were conducted at 25° C. in PBS and recorded automatically at the time of acquisition. The data sets were analyzed by fitting an exponential decay using Prism 6 (GraphPad) to calculate the observed pseudo-first-order rate constants that were converted to second-order rate constants by dividing with the concentration of the excess TCO compound.

Only reactions that showed a minimum second-order rate constant of 500 M⁻¹ s⁻¹ in phosphate-buffered saline at 25° C. are considered suitable for providing the sufficient speed kinetics and therefore, reactions wherein the reaction kinetics was lower were disregarded for the purpose of the method according to the invention.

1. Method for providing a labeled single isomeric chemical entity targeting vector comprising:
a) labeling a first chemical entity having inverse electron demand Diels-Alder cycloaddition reactivity and being conjugated to a pharmaceutic agent, an imaging agent, or a therapeutic agent, with a labeling agent; wherein the first chemical entity is selected from the group consisting of a symmetrical substituted diene wherein at least one of the symmetry planes pass through the nitrogen-nitrogen bonds of at least one tetrazine ring, an unsymmetrical substituted diene, and an isomer-free dienophile; and
b) ligating the labeled first chemical entity obtained in step a) with a second chemical entity having complementary inverse electron demand Diels-Alder cycloaddition reactivity and being conjugated to a targeting vector; wherein the second chemical entity is selected from the group consisting of a symmetrical substituted diene wherein at least one of the symmetry planes pass through the nitrogen-nitrogen bonds of at least one tetrazine ring, an unsymmetrical substituted diene, and an isomer-free dienophile,
wherein the reaction kinetics for the inverse electron demand Diels-Alder cycloaddition between the first and second chemical entities has a minimum rate constant of 500 M⁻¹ s⁻¹ in phosphate-buffered saline (PBS) at 25° C., determined by stopped-flow spectrophotometry,
with the proviso that when the labeling agent in step a) is ⁹⁴Tc, ^{99m}Tc, ²¹¹At, ²²³Ra or ²²⁵Ac the labeling agent is conjugated to an unsymmetrical substituted diene; and
c) oxidizing the ligated labeled targeting vector obtained from step b) at a temperature ranging from 15° C. to 50° C. for up to 60 minutes by adding from 1 to 100 equivalents of chloranil, fluoranil, DDQ or NaNO₂.

2. Method according to claim 1 wherein the symmetrical substituted diene, wherein at least one of the symmetry planes pass through the nitrogen-nitrogen bonds of at least one tetrazine ring, is a tetrazine and the dienophile is a trans-cycloheptene (TCH), a trans-cyclooctene (TCO) or a trans-cyclononene (TCN) derivative.

3. Method according to claim 1 wherein the labeling agent in step a) is a radionuclide or a stable isotope of a corresponding element.

4. Method according to claim 3 wherein the labeling agent is selected from ¹H, ²H, ³H, ¹¹C, ¹²C, ¹³C, ¹⁴C, ¹³N, ¹⁴N, ¹⁵N, ¹⁸F, ¹⁹F, ¹²³I, ¹²⁴I, ¹²⁵J, ¹²⁷J, ¹³¹I, ¹⁵O, O¹⁶, ¹⁷O, ¹⁸O, ⁴³Sc, ⁴⁴Sc, ⁴⁵Sc, ⁴⁵Ti, ⁴⁶Ti, ⁴⁷Ti, ⁴⁸Ti, ⁴⁹Ti, ⁵⁰Ti, ⁵⁵Co, ⁵⁸mCo, ⁵⁹Co, ⁶⁰Cu, ⁶¹Cu, ⁶³Cu, ⁶⁴Cu, ⁶⁵Cu, ⁶⁷Cu, ⁶⁷Ga, ⁶⁸Ga, ⁶⁹Ga, ⁷¹Ga, ⁷⁶Br, ⁷⁷Br, ⁷⁹Br, ^{80m}Br, ⁸¹Br, ⁷²As, ⁷⁵As, ⁸⁶Y, ⁸⁹Y, ⁹⁰Y, ⁸⁹Zr, ⁹⁰Zr, ⁹¹Zr, ⁹²Zr, ⁹⁴Zr, ¹⁴⁹Tb, ¹⁵²Tb, ¹⁵⁹Tb, ¹⁶¹Tb, ¹¹¹In, ¹¹³In, ¹¹⁴mIn, ¹¹⁵mIn, ¹⁷⁵Lu, ¹⁷⁷Lu, ¹⁸⁵Re, ¹⁸⁶Re, ¹⁸⁸Re, ²⁰¹Tl, ²⁰³Tl, ²⁰⁵Tl, ²⁰⁶Pb, ²⁰⁷Pb, ²⁰⁸Pb, ²¹²Pb, ²⁰⁹Bi, ²¹²Bi, ²¹³Bi, ³¹P, ³²P, ³³P, ³²S, ³⁵S, ⁴⁵Sc, ⁴⁷Sc, ⁸⁴Sr, ⁸⁶Sr, ⁸⁷Sr, ⁸⁸Sr, ⁸⁹Sr, ¹⁶⁵Ho, ¹⁶⁶Ho, ¹⁵⁶Dy, ¹⁵⁸Dy, ¹⁶⁰Dy, ¹⁶¹Dy, ¹⁶²Dy, ¹⁶³Dy, ¹⁶⁴Dy, ¹⁶⁵Dy, ²²⁷Th, ²³²Th, ⁵¹Cr, ⁵²Cr, ⁵³Cr, ⁵⁴Cr, ⁷³Se, ⁷⁴Se, ⁷⁵Se, ⁷⁶Se, ⁷⁷Se, ⁷⁸Se, ⁸⁰Se, ⁸²Se, ⁹⁴Tc, ^{99m}Tc, ¹⁰³Rh, ¹⁰³mRh, ¹¹⁹Sb, ¹²¹Sb, ¹²³Sb, ¹³⁵La, ¹³⁸La, ¹³⁹La, ¹⁶²Er, ¹⁶⁴Er, ¹⁶⁵Er, ¹⁶⁶Er, ¹⁶⁷Er, ¹⁶⁸Er, ¹⁷⁰Er, ¹⁹³mPt, ¹⁹⁵mPt, ¹⁹²Pt, ¹⁹⁴Pt, ¹⁹⁵Pt, ¹⁹⁶Pt, ¹⁹⁸Pt, ²¹¹At, ²²³Ra, ²²⁵Ac.

5. Method according to claim 1, wherein the targeting vector is an antibody, a nanobody, a polymer, a nanomedicine, a cell, a protein, a peptide, or a small molecule.

6. Method according to claim 1, wherein the oxidant is solid phase supported.

7. Method according to claim 1 wherein the diene is a symmetrical tetrazine with formula Tz1: 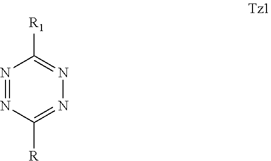
wherein R and R₁ is 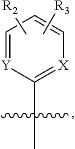
wherein the curly sign indicates the link to the tetrazine; and where R₂ is —H or (i) an isotope labeling agent directly connected to the aromatic ring; or (ii) an isotope labeling agent connected to the aromatic ring via a linker, said linker being selected from the group consisting of —(CH₂)ₙ, LO(CH₂)ₙ, -LNH(CH₂)ₙ, -LCONH(CH₂)ₙ, -LNHCO(CH₂)ₙ, where L is —(CH₂)ₘ or —(CH₂CH₂O)ₘ, where n and m are independently selected from 1-25; or (iii) an isotope labeling agent that is chelated through a chelator selected from: 1,4,7,10-tetraazacyclododecane-N,N′N′,N″-tetraacetic acid (DOTA), N,N′-bis(2-hydroxy-5-(carboxyethyl)benzyl)ethylenediamine N,N′-diacetic acid (HBED-CC), 14,7-triazacyclononane-1,4,7-triacetic acid (NOTA), 2-(4.7-bis(carboxymethyl)-1,4,7-triazonan-1-yl)pentanedioic acid (NODAGA), 2-(4,7,10-tris(carboxymethyl)-1,4,7,10-tetraazacyclododecan-1-yl)pentanedioic acid (DOTAGA), 14,7-triazacyclononane phosphinic acid (TRAP), 14,7-triazacyclononane-1-methyl(2-carboxyethyl)phosphinic acid-4,7-bis(methyl(2-hydroxymethyl)phosphinic acid (NOPO), 3,6,9,15-tetraazabicyclo9.3.1.pentadeca-1 (15),11,13-triene-3,6,9-triacetic acid (PCTA), N-(5-acetyl (hydroxy)aminopentyl-N-(5-(4-(5-aminopentyl)(hydroxy)amino-4-oxobutanoyl)amino)pentyl-N-hydroxysuccinamide (DFO), diethylenetriaminepentaacetic acid (DTPA), trans-cyclohexyl-diethylenetriaminepentaacetic acid (CHX-DTPA), 1-oxa-4,7,10-triazacyclododecane-4,7,10-triacetic acid (OXO-Do3A), p-isothiocyanatobenzyl-DTPA (SCN-BZ-DTPA), 1-(p-isothiocyanatobenzyl)-3-methyl-DTPA (1B3M), 2-(p-isothiocyanatobenzyl)-4-methyl-DTPA (1M3B), and 1-(2)-methyl-4-isocyanatobenzyl-DTPA (MX-DTPA) connected to the aromatic ring through a linker, said linker being selected from the group consisting of —(CH₂)ₙ, -LO(CH₂)ₙ, -LNH(CH₂)ₙ, -LCONH(CH₂)ₙ, -LNHCO(CH₂)ₙ, where L is —(CH₂)ₘ or —(CH₂CH₂O)ₘ, and n and m are independently selected from 1-25;
wherein, when R₂ is either (i) or (ii) the isotope labeling agent is selected from the group consisting of: ¹H, ²H, ³H, ¹¹C, ¹²C, ¹³C, ¹⁴C, ¹³N, ¹⁴N, ¹⁵N, ¹⁸F, ¹⁹F, ¹²³I, ¹²⁴I, ¹²⁵I, ¹²⁷I, ¹³¹I, ²¹¹At, ¹⁵O, ¹⁶O, ¹⁷O, ¹⁸O, ⁴³Sc, ⁴⁴Sc, ⁴⁵Sc, ⁴⁵Ti, ⁴⁶Ti, ⁴⁷Ti, ⁴⁸Ti, ⁴⁹Ti, ⁵⁰Ti, ⁵⁵Co, ⁵⁸mCo, ⁵⁹Co, ⁶⁰Cu, ⁶¹Cu, ⁶³Cu, ⁶⁴Cu, ⁶⁵Cu, ⁶⁷Cu, ⁶⁷Ga, ⁶⁸Ga, ⁶⁹Ga, ⁷¹Ga, ⁷⁶Br, ⁷⁷Br, ⁷⁹Br, ^{80m}Br, ⁸¹Br, ⁷²As, ⁷⁵As, ⁸⁶Y, ⁸⁹Y, ⁹⁰Y, ⁸⁹Zr, ⁹⁰Zr, ⁹¹Zr, ⁹²Zr, ⁹⁴Zr, ¹⁴⁹Tb, ¹⁵²Tb, ¹⁵⁹Tb, ¹⁶¹Tb, ¹¹¹In, ¹¹³In, ¹¹⁴ min, ¹¹⁵ min, ¹⁷⁵Lu, ¹⁷⁷Lu, ¹⁸⁵Re, ¹⁸⁶Re, ¹⁸⁸Re, ²⁰¹Tl, ²⁰³Tl, ²⁰⁵Tl, ²⁰⁶Pb ²⁰⁷Pb, ²⁰⁸Pb, ²¹²Pb ²⁰⁹Bi, ²¹²Bi, 2 ¹³Bi, ³¹P, ³²P, ³³P, ³²S, ³⁵S, ⁴⁵Sc, ⁴⁷Sc, ⁸⁴Sr, ⁸⁶Sr, ⁸⁷Sr, ⁸⁸Sr, ⁸⁹Sr, ¹⁶⁵Ho, ¹⁶⁶Ho, ¹⁵⁶Dy, ¹⁵⁸Dy, ¹⁶⁰Dy, ¹⁶¹Dy, ¹⁶²Dy, ¹⁶³Dy, ¹⁶⁴Dy, ¹⁶⁵Dy, ²²⁷Th, ²³²Th, ⁵¹Cr, ⁵²Cr, ⁵³Cr, ⁵⁴Cr, ⁷³Se, ⁷⁴Se, ⁷⁵Se, ⁷⁶Se, ⁷⁷Se, ⁷⁸Se, ⁸⁰Se, ⁸²Se, ⁹⁴Tc, ^{99m}Tc, ¹⁰³Rh, ¹⁰³mRh, ¹¹⁹Sb, ¹²¹Sb, ¹²³Sb, ¹³⁵La, ¹³⁸La, ¹³⁹La, ¹⁶²Er, ¹⁶⁴Er, ¹⁶⁵Er, ¹⁶⁶Er, ¹⁶⁷Er, ¹⁶⁸Er, ¹⁷⁰Er, ¹⁹³mPt, ¹⁹⁵mPt, ¹⁹²Pt, ¹⁹⁴Pt, ¹⁹⁵Pt, ¹⁹⁶Pt, ¹⁹⁸Pt,
and wherein X and Y are independently selected from: —CH and —N—;
and wherein R₃ is independently selected from H or a moiety selected from the group consisting of a hydroxy group, a sulfonamide, a carboxyl group, a sulfonyl group, amine, a substituted amine with 1-5 polyethylene glycol unit(s), a —(O—CH₂—CH₂)ₙ—OCH₂—COOH, and n is selected from 1-5; or Methyl, Ethyl, Propyl, optionally substituted heteroaryl, and optionally substituted arylalkyl; wherein optionally substituted in relation to said substituted amine means one or more substituents selected from, a halogen, a hydroxy group, a sulfonamide, a carboxyl group, a sulfonyl group, amine, (C1-C10) alkyl, (C2-C10)alkenyl, (C2-C10)alkynyl, (C1-C10)alkylene, (C1-C10)alkoxy, (C2-C10)dialkylamino, (C1-C10)alkylthio, (C2-C10)heteroalkyl, (C2-C10)heteroalkylene, (C3-30 C10)cycloalkyl, (C3-C10)heterocycloalkyl, (C3-10)cycloalkylene, (C3-C10)heterocycloalkylene, (C1-C10)haloalkyl, (C1-C10)perhaloalkyl, (C2-C10)-alkenyloxy, (C3-C10)-alkynyloxy, aryloxy, arylalkyloxy, heteroaryloxy, heteroarylalkyloxy, (C1-C6)alkyloxy-(C1-C4)alkyl, optionally substituted aryl, optionally substituted heteroaryl, and optionally substituted arylalkyl; wherein optionally substituted means one or more substituents selected from a halogen, a hydroxy group, a sulfonamide, a carboxyl group, a sulfonyl group, amine, a substituted amine with 1-5 polyethylene glycol unit(s), a —(O—CH₂—CH₂)ₙ—OCH₂—COOH, and n is selected from 1-5; or H, Methyl, Ethyl, Propyl, optionally substituted heteroaryl, and optionally substituted arylalkyl; wherein optionally substituted in relation to said substituted amine means one or more substituents selected from a halogen, a hydroxy group, a sulfonamide, a carboxyl group, a sulfonyl group, and amine;
and wherein R and R₁ are identical or differs only in the isotope number of the labelling agent.

8. Method according to claim 7 wherein the symmetrical tetrazine is selected from:

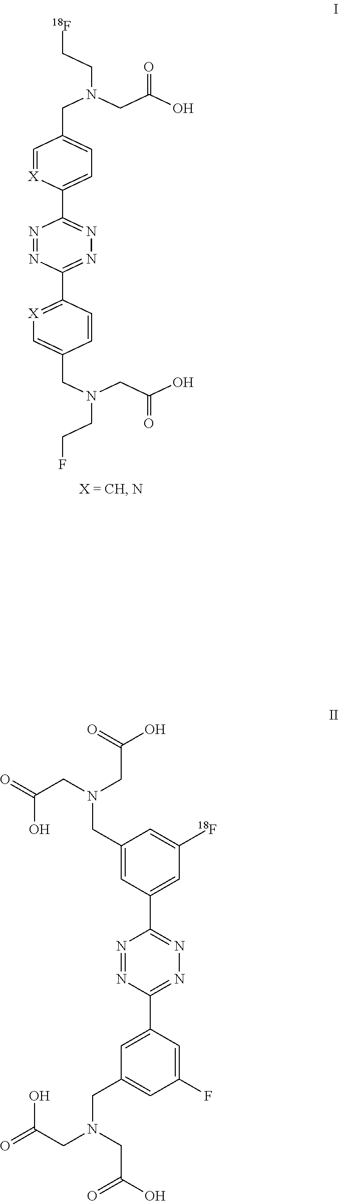

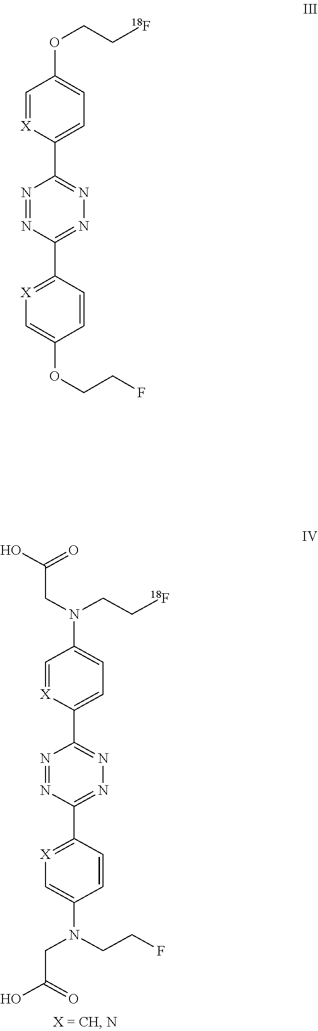

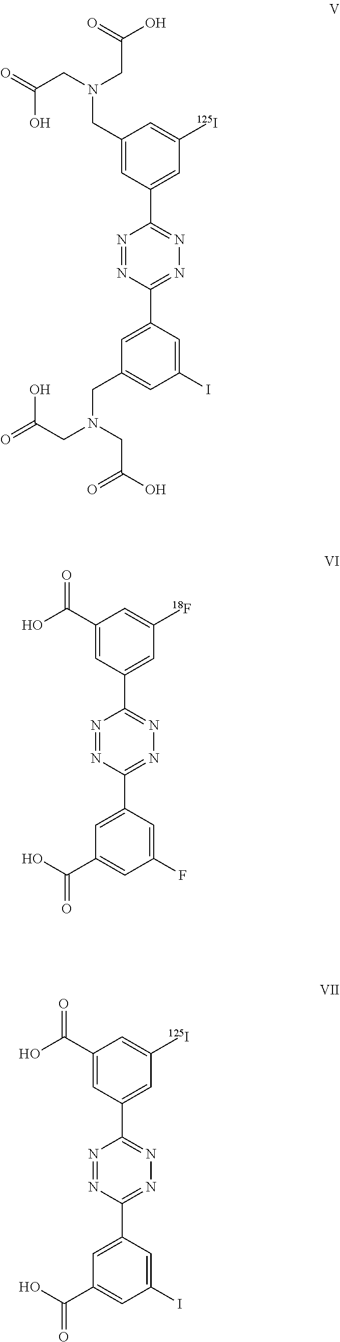 [SMILES: O=C(O)CN(CC(=O)O)Cc1cc(I)cc(-c2nnc(-c3cc([125I])cc(CN(CC(=O)O)CC(=O)O)c3)nn2)c1 ; O=C(O)c1cc(F)cc(-c2nnc(-c3cc([18F])cc(C(=O)O)c3)nn2)c1 ; O=C(O)c1cc(I)cc(-c2nnc(-c3cc([125I])cc(C(=O)O)c3)nn2)c1]

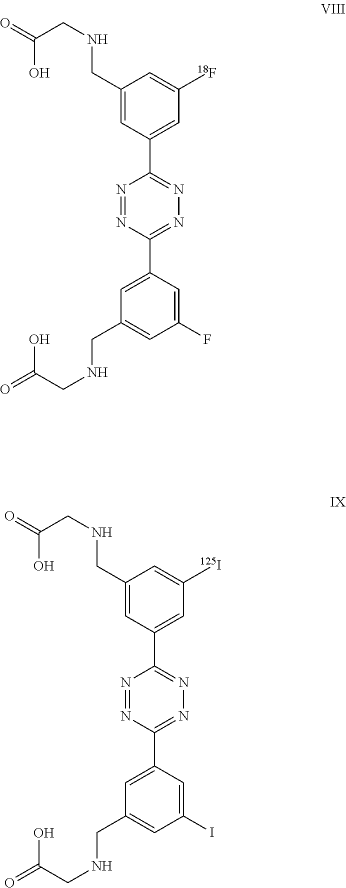 [SMILES: O=C(O)CNCc1cc(F)cc(-c2nnc(-c3cc([18F])cc(CNCC(=O)O)c3)nn2)c1 ; O=C(O)CNCc1cc(I)cc(-c2nnc(-c3cc([125I])cc(CNCC(=O)O)c3)nn2)c1]

9. Method according to claim 1 wherein the diene is an unsymmetrical tetrazine of formula Tz2: 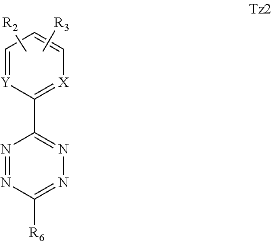
wherein R₄ is —H or (i) an isotope labeling agent directly connected to the aromatic ring or (ii) an isotope labeling agent connected to the aromatic ring via a linker, said linker being selected from the group consisting of —(CH₂)ₙ, -LO(CH₂)ₙ, -LNH(CH₂)ₙ, -LCONH(CH₂)ₙ, -LNHCO(CH₂)ₙ, where L is —(CH₂)ₘ or —(CH₂CH₂O)ₘ, where n and m are independently selected from 1-25, or (iii) an isotope labeling agent that is chelated through a chelator selected from: 1,4,7,10-tetraazacyclododecane-N,N′,N′,N″-tetraacetic acid (DOTA), N,N′-bis(2-hydroxy-5-(carboxyethyl)benzyl)ethylenediamine N,N′-diacetic acid (HBED-CC), 14,7-triazacyclononane-1,4,7-triacetic acid (NOTA), 2-(4.7-bis(carboxymethyl)-1,4,7-triazonan-1-yl)pentanedioic acid (NODAGA), 2-(4,7,10-tris(carboxymethyl)-1,4,7,10-tetraazacyclododecan-1-yl)pentanedioic acid (DOTAGA), 14,7-triazacyclononane phosphinic acid (TRAP), 14,7-triazacyclononane-1-methyl(2-carboxyethyl)phosphinic acid-4,7-bis(methyl(2-hydroxymethyl)phosphinic acid (NOPO), 3,6,9,15-tetraazabicyclo9.3.1.pentadeca-1 (15),11,13-triene-3,6,9-triacetic acid (PCTA), N′-(5-acetyl (hydroxy)aminopentyl-N-(5-(4-(5-aminopentyl)(hydroxy)amino-4-oxobutanoyl)amino)pentyl-N-hydroxysuccinamide (DFO), diethylenetriaminepentaacetic acid (DTPA), trans-cyclohexyl-diethylenetriaminepentaacetic acid (CHX-DTPA), 1-oxa-4,7,10-triazacyclododecane-4,7,10-triacetic acid (OXO-Do3A), p-isothiocyanatobenzyl-DTPA (SCN-BZ-DTPA), 1-(p-isothiocyanatobenzyl)-3-methyl-DTPA (1B3M), 2-(p-isothiocyanatobenzyl)-4-methyl-DTPA (1M3B), and 1-(2)-methyl-4-isocyanatobenzyl-DTPA (MX-DTPA) and connected to the aromatic ring through a linker, said linker being selected from the group consisting of —(CH₂)ₙ, -LO(CH₂)ₙ, -LNH(CH₂)ₙ, -LCONH(CH₂)ₙ, -LNHCO(CH₂)ₙ, where L is —(CH₂)ₘ or —(CH₂CH₂O)ₘ, and n and m are independently selected from 1-25; wherein when R₄ is (i), (ii) or (iii) the isotope labeling agent is selected from the group consisting of: ¹H, ²H, ³H, ¹¹C, ¹²C, ¹³C, ¹⁴C, ¹³N, ¹⁴N, ¹⁵N, ¹⁸F, ¹⁹F, ¹²³I, ¹²⁴I, ¹²⁵I, ¹²⁷I, ¹³¹I, ²¹¹At, ¹⁵O, ¹⁶O, ¹⁷O, ¹⁸O, ⁴³Sc, ⁴⁴Sc, ⁴⁵Sc, ⁴⁵Ti, ⁴⁶Ti, ⁴⁷Ti, ⁴⁸Ti, ⁴⁹Ti, ⁵⁰Ti, ⁵⁵Co, ⁵⁸mCo, ⁵⁹Co, ⁶⁰Cu, ⁶¹Cu, ⁶³Cu, ⁶⁴Cu, ⁶⁵Cu, ⁶⁷Cu, ⁶⁷Ga, ⁶⁸Ga, ⁶⁹Ga, ⁷¹Ga, ⁷⁶Br, ⁷⁷Br, ⁷⁹Br, ^{80m}Br, ⁸¹Br, ⁷²As, ⁷⁵As, ⁸⁶Y, ⁸⁹Y, ⁹⁰Y, ⁸⁹Zr, ⁹⁰Zr, ⁹¹Zr, ⁹²Zr, ⁹⁴Zr, ¹⁴⁹Tb, ¹⁵²Tb, ¹⁵⁹Tb, ¹⁶¹Tb, ¹¹¹In, ¹¹³In, ¹¹⁴mIn, ¹¹⁵mIn, ¹⁷⁵Lu, ¹⁷⁷Lu, ¹⁸⁵Re, ¹⁸⁶Re, ¹⁸⁸Re, ²⁰¹Tl, ²⁰³Tl, ²⁰⁵Tl, ²⁰⁶Pb, ²⁰⁷Pb, ²⁰⁸Pb, ²¹²Pb, ²⁰⁹Bi, ²¹²Bi, ²¹³Bi, ³¹P, ³²P, ³³P, ³²S, ³⁵S, ⁴⁵Sc, ⁴⁷Sc, ⁸⁴Sr, ⁸⁶Sr, ⁸⁷Sr, ⁸⁸Sr, ⁸⁹Sr, ¹⁶⁵Ho, ¹⁶⁶Ho, ¹⁵⁶Dy, ¹⁵⁸Dy, ¹⁶⁰Dy, ¹⁶¹Dy, ¹⁶²Dy, ¹⁶³Dy, ¹⁶⁴Dy, ¹⁶⁵Dy, ²²⁷Th, ²³²Th, ⁵¹Cr, ⁵²Cr, ⁵³Cr, ⁵⁴Cr, ⁷³Se, ⁷⁴Se, ⁷⁵Se, ⁷⁶Se ⁷⁷Se ⁷⁸Se ⁸⁰Se ⁸²Se ⁹⁴Tc, ^{99m}Tc, ¹⁰³Rh, ¹⁰³mRh, ¹¹⁹Sb, ¹²¹Sb, ¹²³Sb, ¹³⁵La, ¹³⁸La ¹³⁹La ¹⁶²Er, ¹⁶⁴Er, ¹⁶⁵Er ¹⁶⁶Er, ¹⁶⁷Er, ¹⁶⁸Er, ¹⁷⁰Er, ¹⁹³mPt, ¹⁹⁵mPt, ¹⁹²Pt, ¹⁹⁴Pt, ¹⁹⁵Pt, ¹⁹⁶Pt, ¹⁹⁸Pt, ²¹¹At, ²²³Ra, ²²⁵Ac,
and wherein X and Y are independently selected from: —CH— and —N—;
and wherein R₆ is H, or 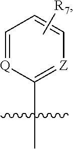
wherein Q and Z are independently selected from: —CH— and —N— and wherein the curly sign indicates the link to the tetrazine;
and wherein R₅ and R₇ are independently selected from H or a moiety selected from the group consisting of a hydroxy group, a sulfonamide, a carboxyl group, a sulfonyl group, amine, a substituted amine with 1-5 polyethylene glycol unit(s), a —(O—CH₂—CH₂)—OCH₂—COOH, and n is selected from 1-5; or methyl, ethyl, propyl, optionally substituted heteroaryl, and optionally substituted arylalkyl; wherein optionally substituted in relation to said substituted amine means one or more substituents selected from a halogen, a hydroxy group, a sulfonamide, a carboxyl group, a sulfonyl group, amine, (C1-C10) alkyl, (C2-C10)alkenyl, (C2-C10)alkynyl, (C1-C10)alkylene, (C1-C10)alkoxy, (C2-C10)dialkylamino, (C1-C10)alkylthio, (C2-C10)heteroalkyl, (C2-C10)heteroalkylene, (C3-C10)cycloalkyl, (C3-C10)heterocycloalkyl, (C3-10)cycloalkylene, (C3-C10)heterocycloalkylene, (C1-C10)haloalkyl, (C1-C10)perhaloalkyl, (C2-C10)-alkenyloxy, (C3-C10)-alkynyloxy, aryloxy, arylalkyloxy, heteroaryloxy, heteroarylalkyloxy, (C1-C6)alkyloxy-(C1-C4)alkyl, optionally substituted aryl, optionally substituted heteroaryl, and optionally substituted arylalkyl; wherein optionally substituted means one or more substituents selected from a halogen, a hydroxy group, a sulfonamide, a carboxyl group, a sulfonyl group, amine, a substituted amine with 1-5 polyethylene glycol unit(s), a —(O—CH₂—CH₂)—OCH₂—COOH, and n is selected from 1-5; or H, Methyl, Ethyl, Propyl, optionally substituted heteroaryl, and optionally substituted arylalkyl; wherein optionally substituted in relation to said substituted amine means one or more substituents selected from a halogen, a hydroxy group, a sulfonamide, a carboxyl group, a sulfonyl group, and an amine.

10. Method according to claim 9 wherein the tetrazine is an unsymmetrical tetrazine s selected from:

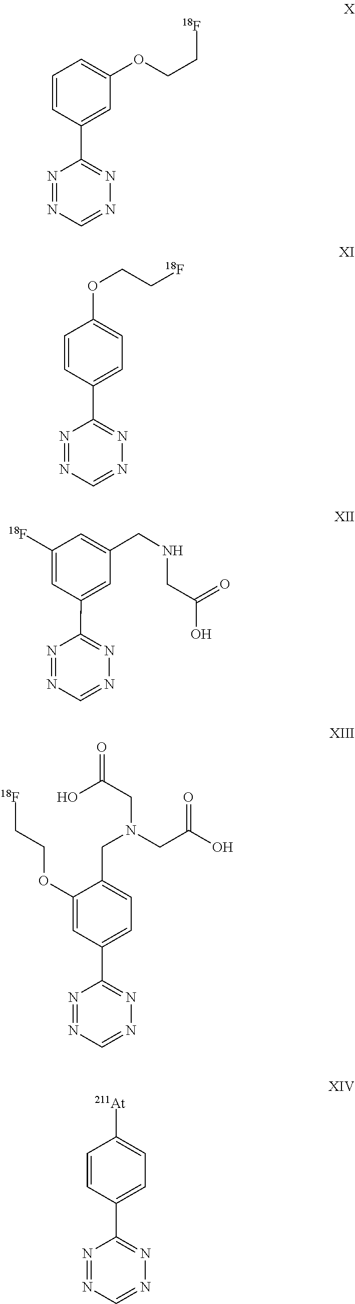

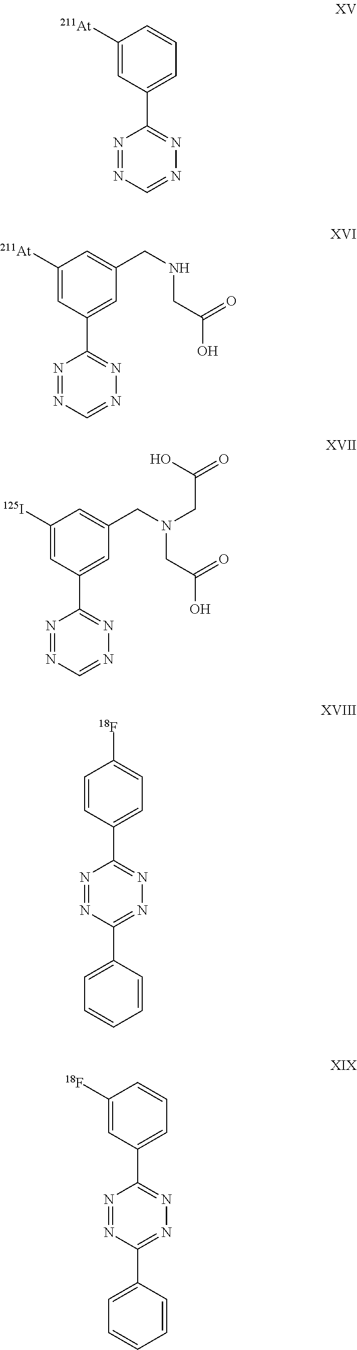

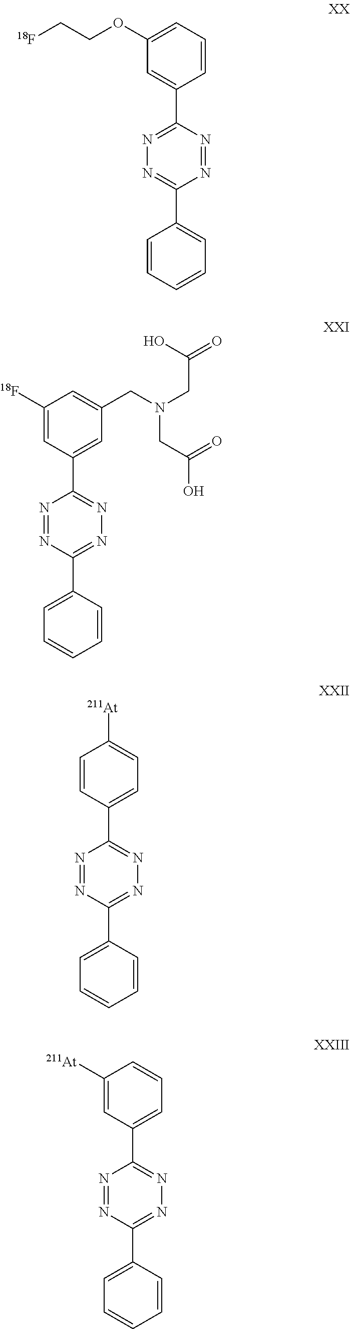

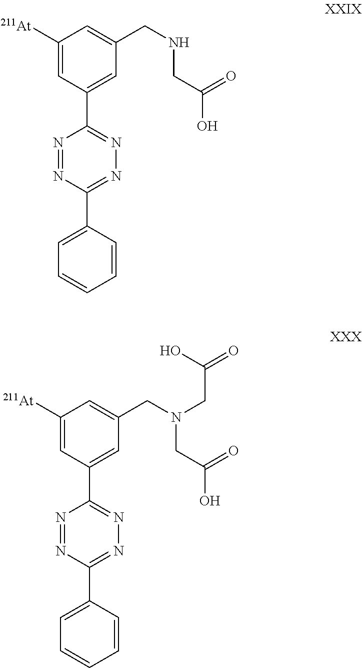

11. Method according to claim 1 wherein the isomer-free dienophile is a trans-cycloheptene (TCH), trans-cyclooctene (TCO) or a trans-cyclononene (TCN) derivative selected from: 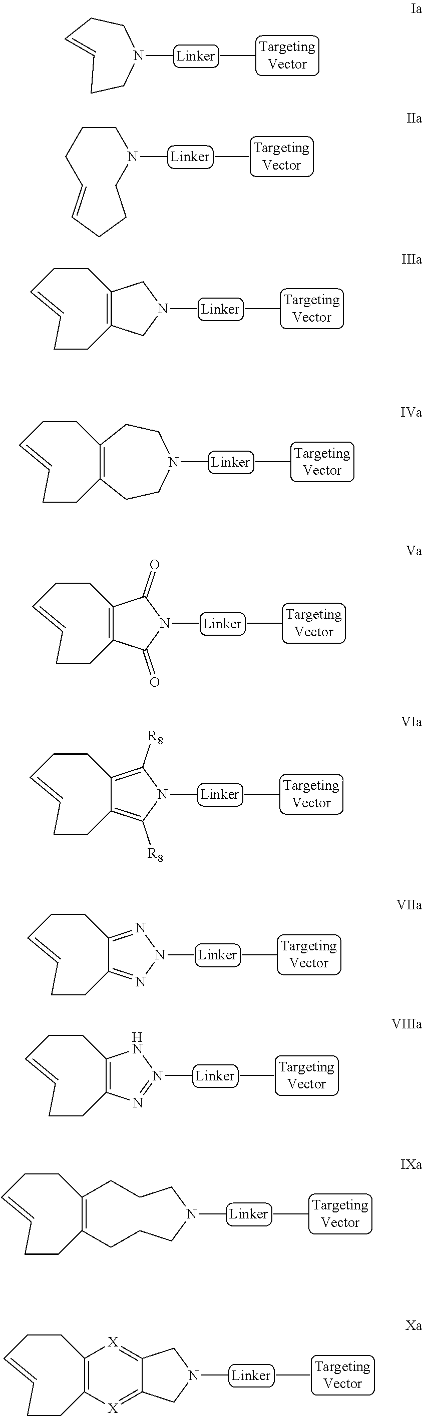 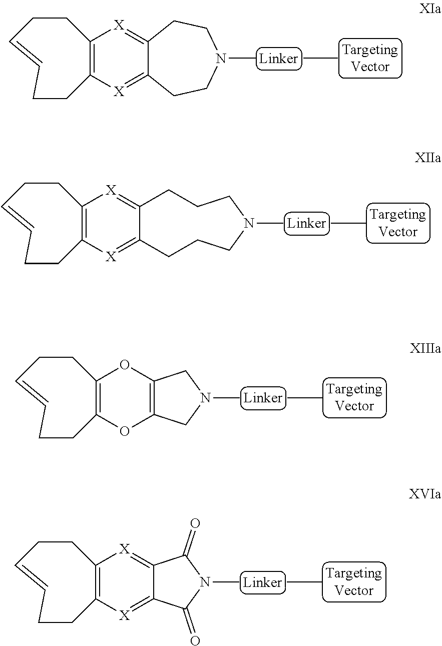 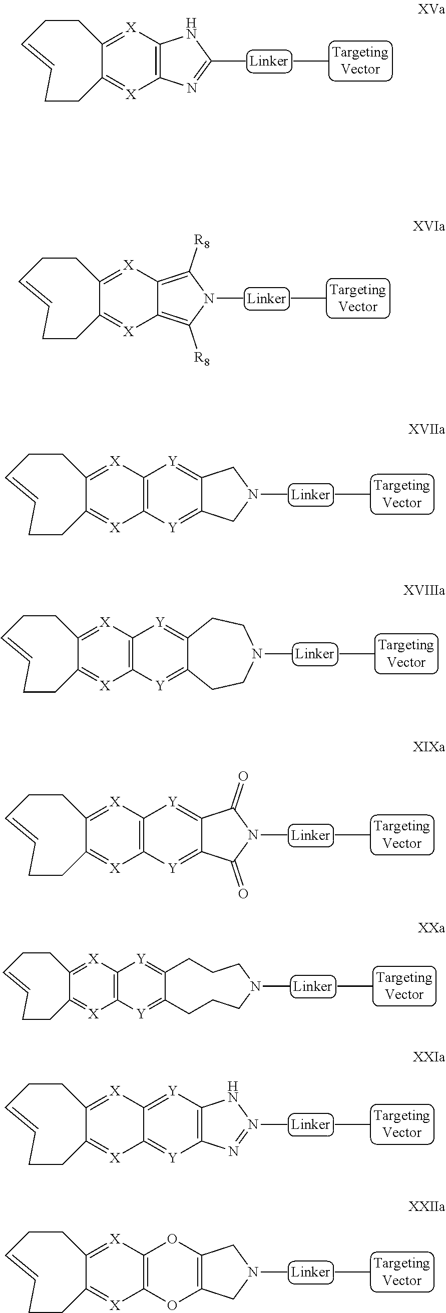
wherein X is N, NO or CR₈; Y is N, NO or CR₈; R₈ is selected from the group consisting of: —H, —F, —OH, —NH₂, —COOH, —COOCH₃, CF₃, —Cl, —CONH₂, CONHCH₃, —CON(CH₃)₂, —CH₂CH₂OH, —CH₂CH₂NH₂, —CHCH₂N(CH₃)₂ and
wherein the linker is selected from the group comprising: —(CH₂)ₙ—(CH₂)ₙNH, (CH₂)ₙCO, (CH₂)ₙO, (CH₂CH₂O)ₙ, (CH₂CH₂O)ₙCH₂CH₂NH, (CH₂CH₂O)ₙCH₂CH₂CO, —CO(CH)₂—CO(CH₂)ₙNH, CO(CH₂)ₙCO, CO(CH₂)ₙO, CO(CH₂CH₂O)ₙCO(CH₂CH₂O)ₙCH₂CH₂NH, CO(CH₂CH₂O)ₙCH₂CH₂CO, COO(CH)₂—COO(CH₂)ₙNH, COO(CH₂)ₙCO, COO(CH₂)ₙO, COO(CH₂CH₂O)ₙCOO(CH₂CH₂O)ₙCH₂CH₂NH, COO(CH₂CH₂O)ₙCH₂CH₂CO, CONH(CH)₂—CONH(CH₂)ₙNH, CONH(CH₂)ₙCO, CONH(CH₂)ₙO, CONH(CH₂CH₂O)ₙ, CONH(CH₂CH₂O)ₙCH₂CH₂NH, CONH(CH₂CH₂O)ₙCH₂CH₂CO, —CONHPhCO, —COOPhCO, —COPhCO, CONHCHMCO, (CH₂)ₙ NHCHMCO, (CH₂)ₙOCONHCHMCO, (CH₂)ₙ NHCHMCO, (CH₂)ₙNHCOCHMNH, (CH₂)OCOCHMNH, (CH₂CH₂O)ₙCH₂CH₂NHCHMCO, (CH₂CH₂O)ₙCH₂CH₂CONHCHMCO, (CH₂CH₂O)ₙCH₂CH₂NHCHMCO, (CH₂CH₂O)ₙCH₂CH₂NHCOCHMNH, (CH₂CH₂O)ₙCOCHMNH, where n is 0-25 and where M is a side chain selected from the group consisting of side chains of the natural amino acids: H, CH₃, CH₂SH, CH₂COOH, CH₂CH₂COOH, CH₂C₆H₅, CH₂C₃H₃N₂, CH(CH₃)CH₂CH₃, (CH₂)₄NH₂, CH₂CH(CH₃)₂, CH₂CH₂SCH₃, CH₂CONH₂, (CH₂)₄NHCOC₄H₅NCH₃, CH₂CH₂CH₂, CH₂CH₂CONH₂, (CH₂)₃NH—C(NH)NH₂, CH₂OH, CH(OH)CH₃, CH₂SeH, CH(CH₃)₂, CH₂C₈H₆N, CH₂C₆H₄OH;
and where the targeting vector is an antibody, a nanobody, a polymer, a nanomedicine, a cell, a protein, a peptide, or a small molecule.

12. Method according to claim 1, wherein when the diene is a symmetrical substituted diene wherein at least one of the symmetry planes pass through the nitrogen-nitrogen bonds of at least one tetrazine ring obtained from a precursor selected from 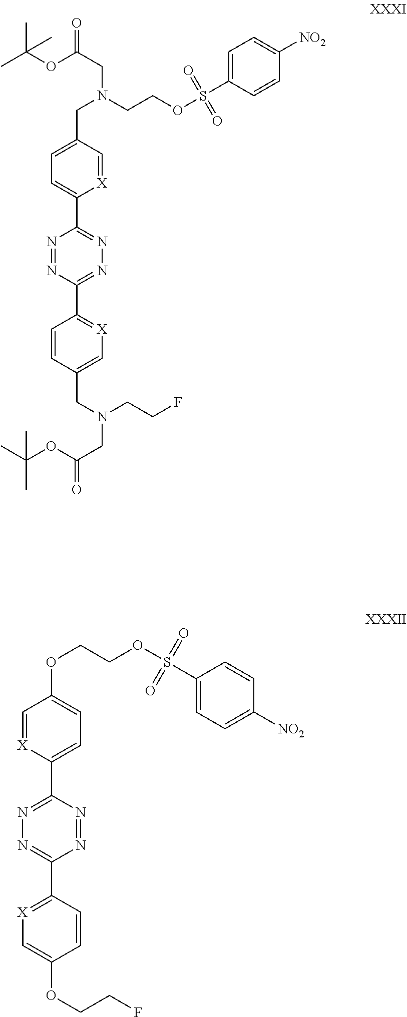 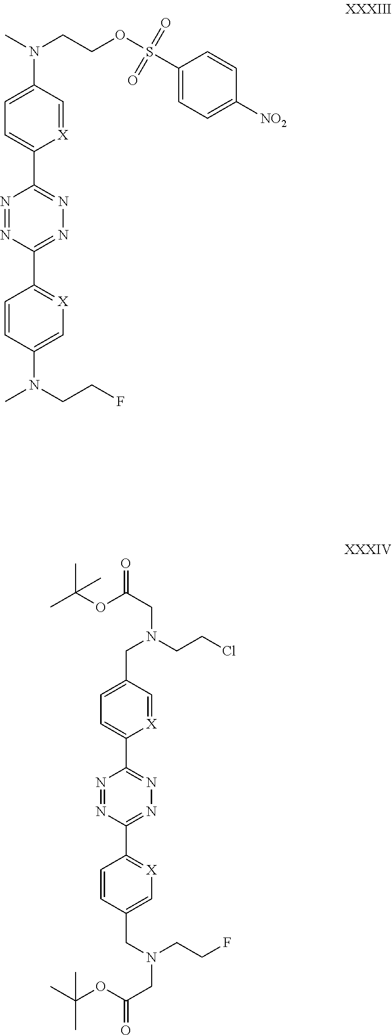 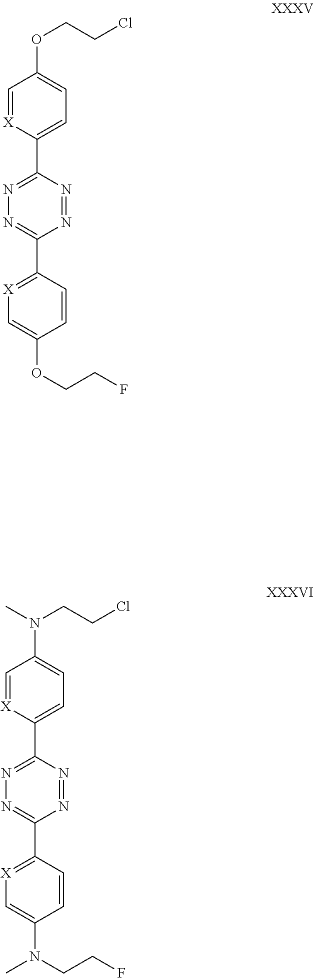 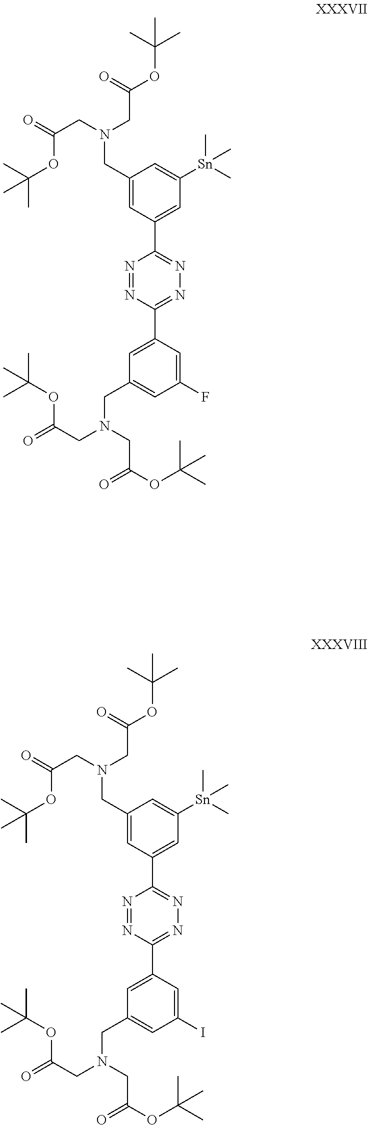 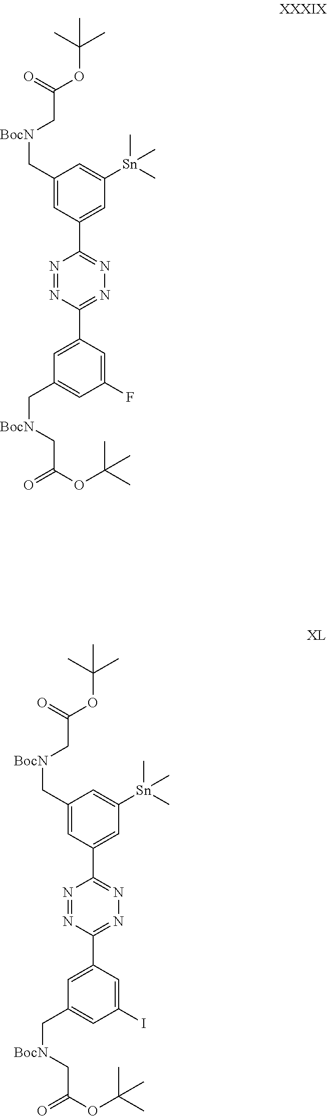 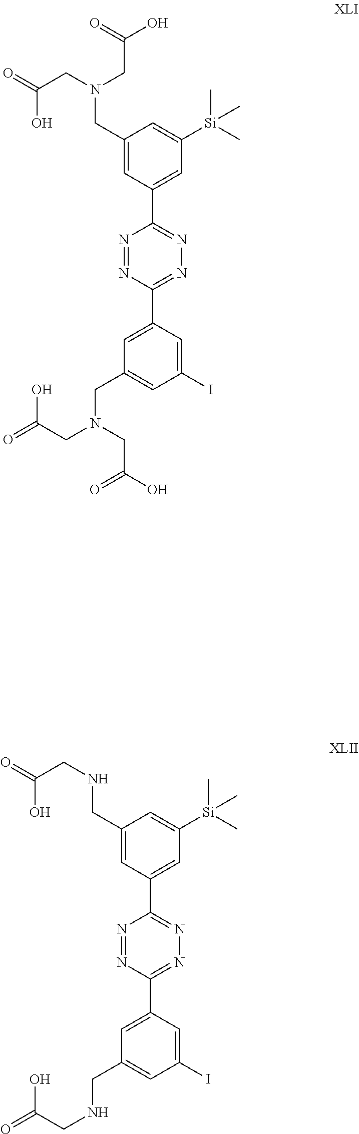 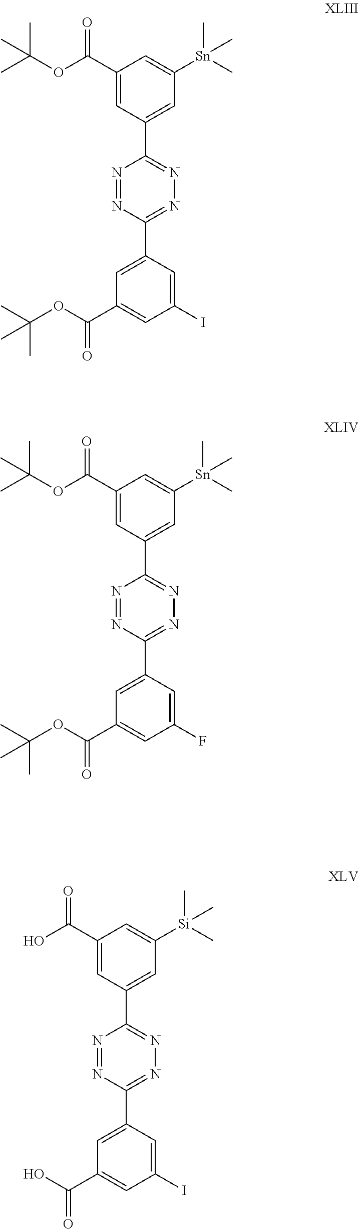
Wherein X is CH or N.

13. Method according to claim 1, wherein when the diene is an unsymmetrical substituted diene obtained from a precursor selected from:

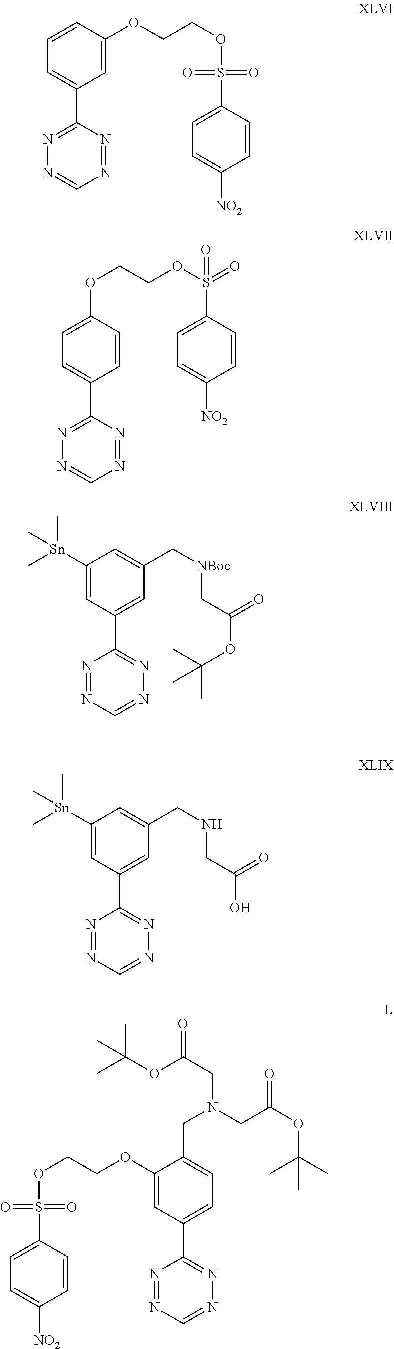

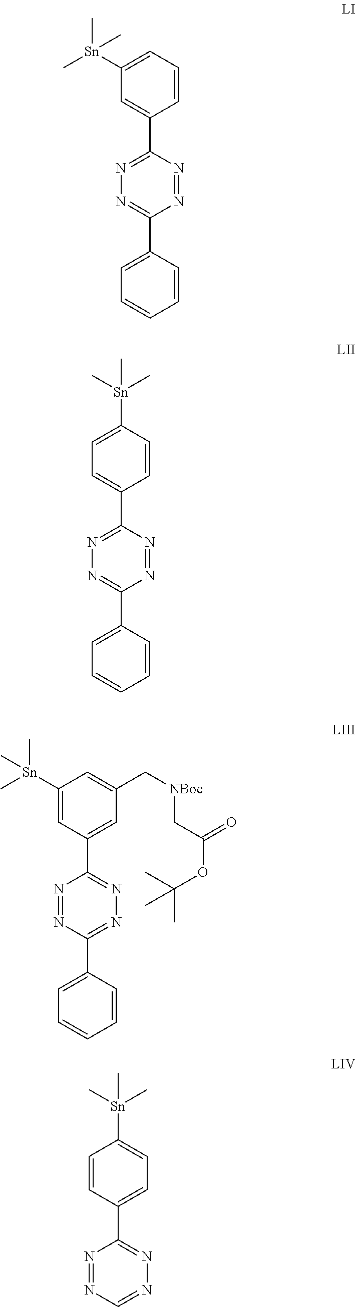

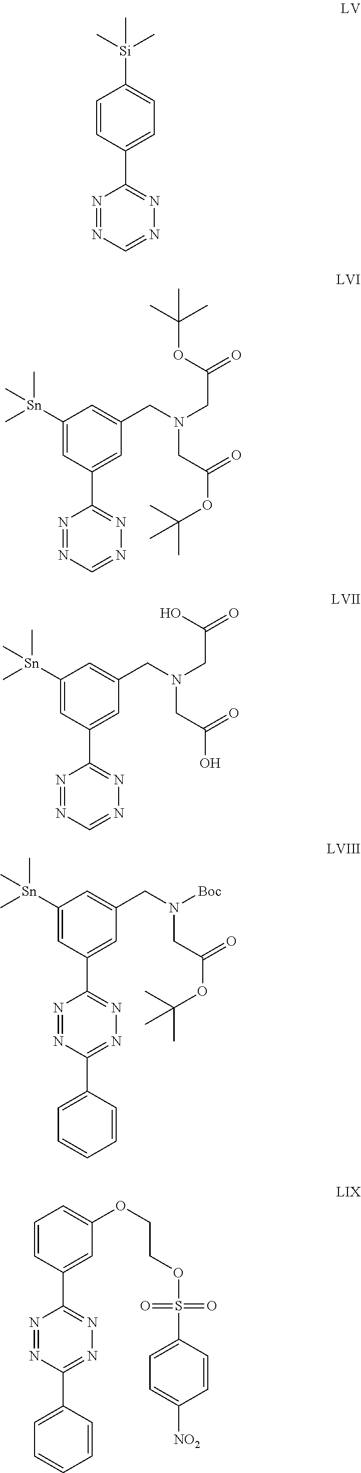 [SMILES: C[Si](C)(C)c1ccc(-c2nncnn2)cc1 ; CC(C)(C)OC(=O)CN(CC(=O)OC(C)(C)C)Cc1cc(-c2nncnn2)c[c]([Sn]([CH3])([CH3])[CH3])c1 ; [CH3][Sn]([CH3])([CH3])[c]1cc(CN(CC(=O)O)CC(=O)O)cc(-c2nncnn2)c1 ; CC(C)(C)OC(=O)CN(Cc1cc(-c2nnc(-c3ccccc3)nn2)c[c]([Sn]([CH3])([CH3])[CH3])c1)C(=O)OC(C)(C)C ; O=[N+]([O-])c1ccc(S(=O)(=O)OCCOc2cccc(-c3nnc(-c4ccccc4)nn3)c2)cc1]

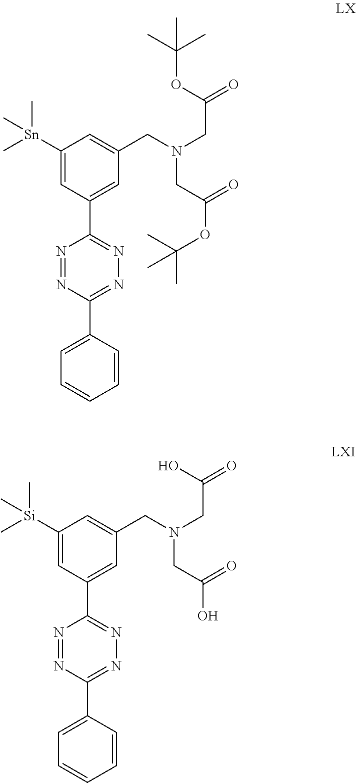 [SMILES: CC(C)(C)OC(=O)CN(CC(=O)OC(C)(C)C)Cc1cc(-c2nnc(-c3ccccc3)nn2)c[c]([Sn]([CH3])([CH3])[CH3])c1 ; C[Si](C)(C)c1cc(CN(CC(=O)O)CC(=O)O)cc(-c2nnc(-c3ccccc3)nn2)c1]

14. Method according to claim 1, wherein the isomer-free dienophile targeting vector is obtained from a precursor selected from: 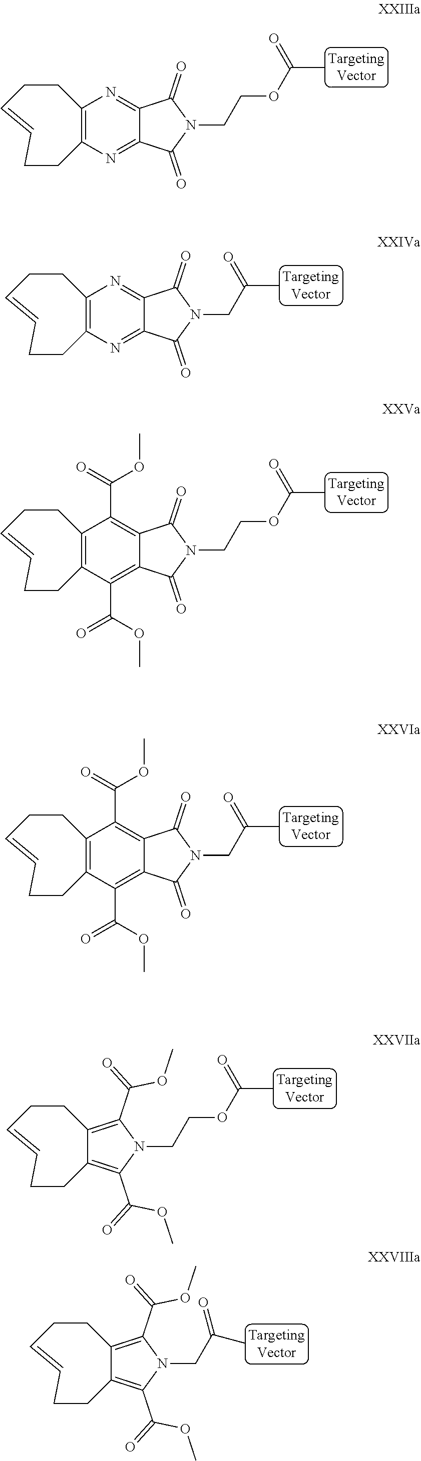 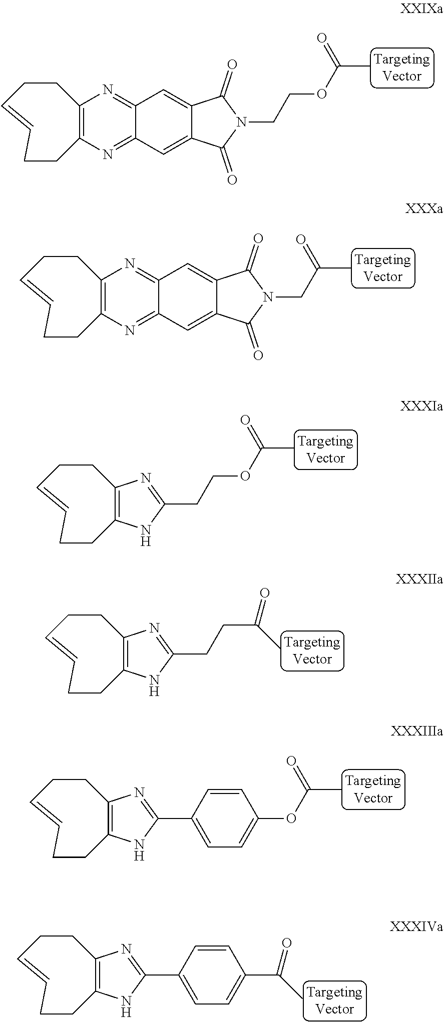 [SMILES: CC(=O)OCCN1C(=O)c2cc3nc4c(nc3cc2C1=O)CC/C=C/CC4 ; CC(=O)CN1C(=O)c2cc3nc4c(nc3cc2C1=O)CC/C=C/CC4 ; CC(=O)OCCc1nc2c([nH]1)CC/C=C/CC2 ; CC(=O)CCc1nc2c([nH]1)CC/C=C/CC2 ; CC(=O)Oc1ccc(-c2nc3c([nH]2)CC/C=C/CC3)cc1 ; CC(=O)c1ccc(-c2nc3c([nH]2)CC/C=C/CC3)cc1]
wherein the targeting vector is an antibody, a nanobody, a polymer, a nanomedicine, a cell, a protein, a peptide, or a small molecule.
